(19) United States
(12) Patent Application Publication
(10) Pub. No.: US 2012/0128683 A1
(43) Pub. Date: May 24, 2012

(54) AUTISM TREATMENT

(76) Inventor: Totada R. Shantha, Stone Mountain, GA (US)

(21) Appl. No.: 13/302,648

(22) Filed: Nov. 22, 2011

(51) Int. Cl. A61K 38/28; A61K 39/395; A61P 25/00; A61K 38/30

(52) U.S. Cl. 424/141.1; 514/5.9; 514/6.5

(57) ABSTRACT

A safe and effective treatment to curtail and cure autism spectrum disorders has been described in this invention using insulin, IGF-1, with multiple known adjuvant therapeutic agents, as well as other pharmaceutical, biochemical, nurticeuticals, and biological agents or compounds delivered through the olfactory mucosal region of the nose and external auditory meatus.

### FIELD OF THE INVENTION

The present invention relates to methods of treating autism and autism related spectrum disorders (ASD), more particularly by administering effective doses of insulin, insulin-like growth factor-I (IGF-1), and other known adjuvant anti-autism therapeutic agents delivered through the olfactory nerves, trigeminal nerve, sphenopalatine ganglion and its connecting braches, and auditory nerve and communicating blood vessels routes to the central nervous system (CNS), to treat ASD in human or mammals.

### BACKGROUND OF THE INVENTION

Autism, also known as autism spectrum disorders (ASD) or grouped as Pervasive Developmental Disorders (PDD) is a developmental disability/disorder that causes problems with social skills and communication which can be mild, moderate or severe, and the signs and symptoms are different for every person who suffers from this disorder. Children with ASD do not follow the typical patterns of child development. Autism is a multifaceted developmental disability that interferes with the normal development of the brain in the areas of social interaction and communication skills. Autism is defined behaviorally because there are no definitive biological markers of the disorder. It classically appears during the first three years of life and is said to be the result of a neurological disorder which affects the functioning of the brain.

Autism is a neuro-developmental disorder characterized by impairments in social relations and communication, repetitive behaviors, abnormal movements, and sensory dysfunction. According to the most recent estimates published by the Centers for Disease Control and Prevention (CDC), it has been reported that approximately 1 in 150 children in the United States suffers from an autistic disorder, and far more males than females suffer from autistic disorders (4.3:1). 2003-2004 statistics show as many as 1.5 million Americans are believed to have some form of autism. Therefore, effective dietary and/or pharmaceutical interventions for ASD could have a major public health impact. There is an unmet need for improved methods of treatment of ASD with various combinations of therapeutic agents that can reduce symptoms associated with autism. The present invention and methods provide these and/or other advantages to existing treatments.

Previously, autism was thought to be rare. Although there are no reliable figures on the prevalence of autism among adults, it has been suggested that in the United States there is anywhere from 58,000-115,000 to 1.5 million children with autism among the 57.6 million children between 1 to 15 years of age. It has been reported that autism occurs in 4 or 5 out of every 10,000 children. The DSM-IV's explicit behavioral criteria have allowed for the identification of more mild cases of child and adult autistic patients.

Among the most common of the PDD, autism affects an estimated 1 in 200 births. Latest studies indicate that 1 in 96 births have some form of ASD. Such a number is on the rise based on statistics from the U.S. Department of Education and other governmental agencies. It is thought that the autism is growing at a rate of 10-17 percent per year. At these rates, the Autism Society of America (ASA) estimates that the prevalence of autism could easily affect 4 million Americans in the next decades. At present, there are 730,000 Americans under age 21 who have been diagnosed with autism (TIME Mar. 8, 2010).

ASD affects all geographic locations. Globally, autism knows no racial, ethnic, or social boundaries; nor family income, lifestyle, and educational levels. One distinguishing point is sex prevalence; males are affected more than the female (4.3:1 ratio).

Recent studies have reported that the exposure to mercury can cause immune, sensory, motor, neurological, and behavioral dysfunctions similar to traits defining or associated with autism. Thimerosal, a preservative added to many vaccines, has become a major source of mercury among children in the United States. The controversy about its contribution to the development of ASD rages even to this day. Exhaustive studies do refute and contest the relationship of mercury in the vaccinations and development of ASD. However, within the first two years of child's life, they may have received a quantity of mercury that exceeded Federal Safety Guidelines. According to the CDC, the recommended immunization schedule in the United States during the 1990s indicated infants may have been exposed to 12.5 μg of ethyl mercury at birth, 62.5 μg of ethyl mercury at 2 months, 50 μg of ethyl mercury at 4 months, 62.5 μg of ethyl mercury at 6 months, and 50.0 μg of ethyl mercury at 18 months, for a total of 237.5 μg of ethyl mercury during the first 18 months of life, if all the thimerosal-containing vaccines were administered. (Redwood L, Bernard S, Brown D, “Predicted mercury concentrations in hair from infant immunizations: cause for concern,” Neurotoxicology; 2001; 22:691-7 and Ball L K, Ball R, Pratt R D., “An assessment of thimerosal use in childhood vaccines” Pediatrics 2001; 107:1147-54). Free radical theory (reactive oxygen species-ROS) and mercury has never been explored. Any compound in your body that contains an oxygen molecule such as hydrogen peroxide can trigger free radical production. This destruction processes happens about 10.000 time every single day in each of the trillions of cell in the body.

Signs and Symptoms of Autism

Mesibov, G. B, Adams, L. W., Schopler, E. (2000). Autism: A Brief History. Psychoanalytic Inquiry, 20:5, 637-647); Rapin, I. (1997) Current Concepts: Autism. The New England journal of Medicine, 337(2), 97-104; are the source of Autism information incorporated herein.

The main symptoms of autism are deficits in sociability, and verbal communications. Contrary to the popular views, children with autism can be affectionate, but on their terms, and without the expected joy and reciprocity. Parents of autistic toddlers may describe them as independent rather than aloof, and may be proud of their child's self-sufficiency. However, anger or depression in adolescence may replace their excessive shyness, fearfulness, anxiety, or rapid changing of mood of a child with autism. If not dealt with early, unprovoked aggressiveness may become a major problem and lead to a need for heavy medication or institutionalization.

Children with autism have problems with communication, social skills, and reacting to the world around them. Not all behaviors will exist in every child. A diagnosis should be made by the child's doctor or other professional with experience in working with children with autism after hearing from the parents and care givers.

Possible signs and symptoms are outlined below. (from The American Speech-Language-Hearing Association)

Communication Message skills:
1. Not speaking or very limited speech
2. Loss of words the child was previously able to say
3. Difficulty expressing basic wants and needs
4. Poor vocabulary development
5. Problems following directions or finding objects that are named
6. Repeating what is said (echolalia)
7. Problems answering questions
8. Speech that sounds different (e.g., “robotic” speech or speech that is high-pitched)

Social Community skills:
1. Poor eye contact with people or objects
2. Poor play skills (pretend or social play)
3. Being overly focused on a topic or objects that interest them
4. Problems making friends
5. Crying, becoming angry, giggling, or laughing for no known reason or at the wrong time
6. Disliking being touched or held
Reacting to the world just about them:
1. Rocking, hand flapping or other movements (self-stimulating movements)
2. Not paying attention to things the child sees or hears
3. Problems dealing with changes in routine
4. Using objects in unusual ways
5. Unusual attachments to objects
6. No fear of real dangers
7. Being either very sensitive or not sensitive enough to touch, light, or sounds (e.g., disliking loud sounds or only responding when sounds are very loud; also called a sensory integration disorder)
8. Feeding difficulties (accepting only select foods, refusing certain food textures)
9. Sleep problems

Although autism may become obvious at infancy with impaired attachment, it is most often identified in toddlers, mostly boys, from 18 to 30 months of age. Parents or pediatricians notice an absence or delay of speech development and a lack of normal interest in others or a regression of early speech and sociability. These autistic traits persist into adulthood but vary from little speech and poor daily living skills throughout life to graduating with a college degree and independent functioning. Adults with autism may be perceived in society as being merely reclusive or they may be given a diagnosis of obsessive-compulsive disorder, schizoid personality, simple schizophrenia, affective disorder, mental retardation, or brain damage.

Other physical differences can sometimes be seen in children with autism such as low-seated ears, a high palate, abnormal head circumference, aberrant earlobes, and a gap between the first and second toes. Evidence for a genetic cause of autism has been supported because studies show that in half of their samples of identical twins, both had autism as compared to none of their non-identical twins.

Autism and Communication Skills

Young children with autism have language disorders as well. At least with young autistic children, comprehension and the communicative use of speech and gestures are typically deficient. The lack of ability to decode rapid sounds that characterize speech results in verbal auditory agnosia or word deafness. Children with this disorder understand little or no language and therefore fail to acquire speech and remain nonverbal. Our invention will help to correct this neurological defect.

Children that are less severely affected by autism may acquire a mixed receptive-aggressive disorder and have better comprehension than expression. Their speech may be described as impoverished, poorly articulated, a grammatical, and sparse. Other children with autism that speak late may progress rapidly from silence or jargon to fluent, clear, and well-formed sentences. However, their speech may still be literal, repetitive, and non-communicative. It is often marked by striking echolalia (involuntary parrot-like repetition of a word or sentence just spoken by another person) or “over-learned scripts” (Rapin IBID). In other words, they may say the appropriate things, but autistic children say it more out of habit rather than actually understanding or planning the appropriateness of what they say. Some autistic children speak nonstop in a high-pitched, singsong, or poorly modulated voice to no one in particular and they focus on their favorite utterances. The present invention ameliorates many of the above signs and symptoms ASD and makes them more functional compared to untreated patients.

Autistic Children at Play

Young autistic children do not know how to play and may manipulate or line up their toys without understanding what the toys actually represent (for example, the idea that dolls represent people), and they do not engage in pretend play which typically starts in other children by the age of two. Pretend play is an important part in building a child's social skills by allowing them to act out and practice situations before they happen.

Some children with autism have particularly long attention spans while doing a repetitive activity alone. However, they are incapable of focusing on an activity involving working with another person. They tend to have temper tantrums if someone tries to make them stop a repetitive activity. Their inability to concentrate, along with other symptoms such as hand flapping, may prevent children from working well with others. A decreased need for sleep and waking up often during the night also separates autistic children from those of the general population.

Autism and its Effect on Cognition

It has been estimated that about 75 percent of persons with autism may be mentally retarded, though the degree of retardation varies from child to child. In the light of recent studies, and the present invention, these results need to be reevaluated after treating with our inventive method. The results of neuropsychological testing reveal an uneven, imbalanced cognitive profile with nonverbal skills generally superior to verbal skills. All their lives, the autistic people tend to have poor insight into what people are thinking with inadequate creativity. On the other hand, a small portion of autistic children excel in music, math, or visual-spatial abilities; despite their other deficits. If these abilities are astounding, autistic children may be known as savants.

Motor Skills and Functioning of Autistics

Some of the ASD children also have increased joint laxity (looseness of the joints), hypotonia (having a decrease in muscle tone), clumsiness, apraxia (loss or impairment of the ability to execute complex coordinated movements without impairment of the muscles or senses), and toe walking. Other motor stereotypes include hand flapping, pacing, spinning, running in circles, twirling a string, tearing paper, drumming, and flipping light switches. This is said to be due to their lack of nystagmus (a repetitive, tremor-like oscillating movement of the eyes) and their increased need for repetitive activity. Oral stereotypes include humming and never-ending repeated questioning. Severe motor stereotypes have been attributed to increased levels of endorphins, which may include self-injurious behavior such as biting, head banging, and gouging. As adults, motor stereotypes are often present in less obvious forms such as finger rubbing.

How Autistics Respond to External Stimuli?

Children with autism may sometimes be hypersensitive or overly aware of new stimuli, and at other times completely unmindful to certain stimuli, such as sounds, tactile stimuli, or pain. Children may sniff their food and have a great aversion to certain tastes, smell or textures. Their visual perception is usually superior to their auditory perception. Children with this symptom may behave unusually at times by covering their ears and staring at some visual display. They may have an amazing visual or auditory memory. In other words, they may have an increased ability to remember order and to perform repetitive, mindless tasks.

Autism and the Incidence of Epileptic Seizures

Seizures are a more frequent occurrence amongst those with autism compared to the general population. By adulthood, about a third of autistic people have had at least two unprovoked epileptic seizures. The probability of epilepsy increases throughout childhood with a peak in adolescence. No etiology has been found, but it has been linked to motor deficits and mental retardation, may be even birth trauma.

Autism Progression and Course of the Disorder

Autism is inclined and liable to improve over time; in some cases children start to acquire language skills and learn to use it to communicate needs and to influence other people. Behavioral deterioration in adolescence may suggest hormonal changes, the difficulty of meeting greater behavioral demands in an increasingly complex social milieu, or depression. Although most autistic patients remain dependent to a specific degree in adulthood, those with enough social skills could find a way to become self-supporting. Rarely do social skills progress sufficient to permit successful marriage.

What Causes Autism?—Etiology

No specific cause for autism has been found; so also no specific treatment has been discovered. Possible prenatal factors that could take part in causing autism include intrauterine rubella, tuberous sclerosis, disorders such as Cornelia de Lange's syndrome, chromosomal abnormalities (such as fragile X, Angel man's syndrome, and even, occasionally Down's syndrome) and high maternal levels of testosterone. Difficulties during birth have been found to play little or no role. But, do not forget parturition stress syndrome (PSS), which is complexly ignored and may a play role in development of ASD; if the birth is prolonged; and, involved the use of the forceps or instruments to deliver the baby. Postnatal conditions frequently cited as being associated with autism include untreated phenylketonuria, infantile spasm, herpes simplex encephalitis, increased levels of testosterone in the mothers during pregnancy, and very rarely, a focal brain lesion such as a neoplasm or some other rare disease or syndrome. Pathophysiology of the ASD is least understood and it could be due to complex cellular mechanisms within the neuropil such as excitotoxicity, free radicle damage, autoimmune type inflammation, up regulation of inflammatory cytokines, necrosis, apoptosis, Synaptic dysregulation and dissimilar growth of various regions of the brain.

Can ASD be an Autoimmune Disease?

The ability or capability of the immune system to distinguish between “self’ and “non-self’ antigens is vital to the functioning of the immune system as a specific defense against invading microorganisms. “Non-self’ antigens (vaccines antigens in autism?) are those antigens entering or present in the body which are detectably different or as foreign from the animal's own constituents; whereas, “self’ antigens are those which, in the healthy animal, are not detectably different or foreign from its own constituents. However, under certain specific conditions, including in certain disease states, an individual's immune system will identify its own constituents as “non-self,” and initiate an immune response against “self’ material, at times causing more damage or discomfort as from an invading microbe or foreign material, and often producing serious illness in an individual. Hence the autoimmune disease results when an individual's immune system attacks his/her own organs or tissues, producing a clinical condition associated with the destruction of that organ or tissue named as autoimmune diseases; as exemplified by diseases such as rheumatoid arthritis, dependent diabetes mellitus, acquired immunodeficiency syndrome (AIDS), hemolytic anemia's, rheumatic fever, Crohn's disease, Guillain-Barre syndrome, psoriasis, thyroiditis, Graves' disease, myasthenia gravis, autism, glomerulonephritis, autoimmune hepatitis, multiple sclerosis, systemic lupus erythematosus, dystrophic epidermolysis bullosa, and the like.

Blocking, neutralizing or inhibiting the immune response or removing its cause in these cases is, therefore, desirable. Autoimmune disease may be the result of a genetic predisposition alone or as the result of the influence of certain exogenous agents such as, viruses, bacteria, or chemical agents, or as the result of the action of both. Some forms of autoimmunity arise as the result of trauma to an area usually not exposed to lymphocytes, such as neural tissue or the lens of the eye. When the tissues in these areas become exposed to lymphocytes, their surface proteins can act as antigens and trigger the production of autoantibodies and cellular immune responses which then begin to destroy those tissues.

ASD are regarded as a disease comprising abnormalities in brain structure and/or function. Studies have indicated that ASD may have an autoimmune component (Warren, et al. 1996, Mol. Chern. Neuropathol. 28: 77-81). Particularly, autoantibodies to myelin basic protein and unique antibodies to an antigenic portion of the measles component of the measles mumps-rubella (MMR) vaccine have been found in the central nervous system of a large proportion of ASD patients evaluated (Singh et al., J. Biomed. 2002, Sci. 9: 359-364). In addition, considerable increases in plasma levels of gamma interferon and IL-12 have been discovered in ASD patients when compared to non-ASD controls (Singh, 1996, J. Neuroimmunol. 66: 143-145). In addition, nitrate, a metabolite of nitrous oxide is considerably higher in autistic children. Elevated nitrate and gamma interferon levels are positively correlated with autism (Sweeten, et al, 2004, Biol. Psychiatry, 55:434-437). However, despite the above laboratory evidence as to the role of autoimmunity in ASD, the etiology of the disease is still a mystery.

If one considers the role of autoimmunity in ASD, the etiology of the disease is treatable with anti-cytokine therapy. Use of insulin and delivering the antibodies (Monoclonal antibodies—mAB) through intranasal ORE as described in this invention could be the answer. However, because autoimmune diseases are complex, often characterized by multiple cytokine abnormalities, effective treatment appears to require the administration or utilization of several agents, each targeting a specific cytokine pathway or its by-product. To meet this need, the methods of treatment of the present invention include use of specific antibodies, pleiotrophic autoimmune inhibitors, antibodies to cytokines and HLA class II antigens, and antigens for the removal of other types of autoantibodies which target cells or DNA with insulin directly delivered to the CNS where the pathological processes is evolving in ASD. Such a therapy can result in the removal, neutralization or inhibition of the pathogenic cytokine(s) from these patients; thereby significantly improve the ASD signs and symptoms using our method of treatment.

In the genetic area, relations have been found between autism and schizophrenia based on duplications and deletions of chromosomes. The researchers have showed that schizophrenia and autism are significantly more common in combination with 1q21.1 deletion syndrome. Studies on autism/schizophrenia relations for chromosome 15 (15q13.3), chromosome 16 (16p13.1) and chromosome 17 (17p12) are inconclusive.

Possible Biomedical Triggers of ASD:

1. Gastrointestinal abnormalities, 2. Immune dysfunctions, 3. Detoxification abnormalities, 4. And/or nutritional deficiencies or imbalances have all been suggested as potential biomedical “triggers” for ASD. It is hard to determine which scenario comes first. These biomedical triggers may play a minor role, if they play any role. If they are really the cause autism, these biological triggers are easy to eliminate and cure the disease, is it not?

Serotonin Reuptake Inhibitors (SSRIs) Antidepressant Use in Pregnancy, And it'S Link to Higher Autism Risk in the New Born

Children whose mothers take Zoloft, Lexapro, Effexor, Paxil, or other antidepressants belonging to a class known as selective serotonin reuptake inhibitors (SSRIs) such as Celexa, Prozac, and Luvox increased the risk of developing autism in their new born, a new study shows. These antidepressants work by increasing available levels of the neurotransmitter serotonin surrounding nerve cells in the brain, boosting the mood. Researchers in California found that women who were prescribed an anti-depression drug in the year before giving birth were twice as likely to have children with an ASD compared to the woman who did not take. Shockingly, the women who were prescribed SSRIs in the first trimester were nearly four times more likely to have a child with autism. Studies indicated that children diagnosed with an ASD have slightly higher blood levels of serotonin, so also family members with autism compared to the families without autistic members. It is estimated that up to 13% of women are treated for depression during pregnancy and the rise in autism rates over the past several decades has roughly paralleled the growth of SSRI use during pregnancy. This theory will for sure question the relationship of ASD to vaccination.

Neurotransmitter Basis of Autism; Signs and Symptoms

Studies of neurotransmitters (at synapses), which are the means by which nerve cells can communicate with one another, suggest that neurotransmitter systems may function differently in autistic patients. Levels of serotonin (which is especially important for controlling some of the behaviors) found in autism is considered abnormal. Researchers have also noted problems with lateralization i.e. problems with communication between the two hemispheres of the brain. Of the brains studied, a significant portion of people with autism had an abnormally large or small cerebellum (Mesibov IBID). All of this evidence and much more that is not mentioned here suggests that neurological problems play a large role in autism. Although the precise nature of the neurological problems has not been pinpointed yet, the fact that autism is related to neurological, biochemical or electrophysiological difficulties in most autistic children cannot be denied. This is not necessarily the best theory of the cause of autism and certainly more than one factor could cause this disease. Our invention which plays a role at synapses of the CNS can reverse the pathophysiology of the disease. There are specific theories of autism dealing with the role opioids play in autism.

How is Autism Diagnosed?

There is not a single test which diagnoses autism. It is important to have your child evaluated by a team of professionals who are well versed with autism. Speech-language pathologists (SLPs), typically as part of a team, may diagnose autism. The team might include pediatricians, neurologists, occupational therapists, physical therapists, and developmental specialists, parents, nannies among others. SLPs play a key role because problems with social skills and communication are often the first symptoms of autism. SLPs should be consulted early in the evaluation process. There are a number of tests and observational checklists available to evaluate children with developmental problems. The most important information or diagnostic clue comes from parents and caregivers who know the child best and can tell the SLP and others all about the child's behavior as they advance in their age.

Correct diagnosis depends on an accurate developmental history paying attention to types of behavior typical of autism and on the evaluation of current functional skills. Cognitive and behavioral evaluation should include an assessment of language (including comprehension, production, voice quality, and the conversational use of speech), sociability (searching for an interest in persons rather than objects, the ability to enjoy an activity someone else suggested, and creative/imaginative play), and the patient's choice of activities (such as new rather than repetitive activities) (Rapin, I. (1997). Current Concepts: Autism. The New England journal of Medicine, 337(2), 97-104).

Using an imaging helmet, researchers discovered that there is lag time of a faction of second which can cascade into a major obstacle in speaking and understanding people (presented at radiological society of North America meeting in Chicago in 2008). Autistic children took a bit longer than normal to understand each syllable which may contribute their problems in communication skill. Finding biomarkers, like the brain waves that enable for earlier diagnosis and treatment, could be an important tool that we researchers want to have. The brain wave study used in this noninvasive technology is called magneto encephalography, MEG for short. It measures magnetic fields generated by electrical currents in brain nerve cells and records brain activity in real time. Researchers at The Children's Hospital of Philadelphia had 64 autistic children, ages 6 to 15; listen through head phones to a series of rapid beeps. In autistic children, response to each sound was delayed by one-fiftieth of a second. “We tend to speak: at four syllables per second,” said Timothy Roberts, the study's lead author. If an autistic brain “is slow in processing a change in a syllable . . . it could easily get to the point of being overloaded.” (Atlanta journal and Constitution 12-1-08). Our invention will overcome this delay to some extent or all of it in children with autism and facilitate response to each sound and improve the communication skills due to processing like normal people CNS.

ASD is a neuro-physiological disorder, which is expressed in both a loss of limb movement and speech. ASD therapies include occupational (at home and within the school setting), and physical therapy and speech therapy. Occupational therapy to improve the fine and gross motor skills by teaching activities include: dressing, toilet training, grooming, buttoning, fine motor and visual skills that assist in writing and scissor use, gross motor coordination to help the individual ride a bike or walk properly, and visual perceptual skills needed for reading and writing.

In order to increase reliability of diagnosing autism; researchers could use the DSM-IV or other such autism-specific diagnostic methodology. According to the Autism Society of America (ASA), autism is generally characterized as one of five disorders coming under the umbrella of Pervasive Developmental Disorders (PDD), a category of neurological disorders characterized by severe and pervasive impairment in several areas of development, including social interaction and communications skills (DSM-IV-TR). The five disorders under PDD are:
I. Autistic Disorder: It is a disorder of neural development characterized by impaired social interaction and communication, and by restricted and repetitive behavior.
II. Asperger's Disorder is an autism spectrum disorder that is characterized by significant difficulties in social interaction, along with restricted and repetitive patterns of behavior and interests. It differs from other autism spectrum disorders by its relative preservation of linguistic and cognitive development.
III. Childhood Disintegrative Disorder (CDD): CDD, also known as Heller's syndrome and disintegrative psychosis, is a rare condition characterized by late onset (>3 years of age) of developmental delays in language, social function, and motor skills. CDD has some similarity to autism, and is sometimes considered a low-functioning form of it.
IV. Rett's Disorder or Rett syndrome is a neuro developmental disorder of the grey matter of the brain that affects only females. The clinical features include small hands and feet and a deceleration of the rate of head growth (including microcephaly in some). Repetitive hand movements, such as wringing and/or repeatedly putting hands into the mouth, are also noted. People with Rett syndrome are prone to gastrointestinal disorders and up to 80% have seizures. They typically have no verbal skills, and about 50% of individuals affected are not ambulatory. Scoliosis, growth failure, and constipation are very common and can be problematic.
V. Pervasive Developmental Disorder-Not Otherwise Specified (PDD-NOS) is a pervasive developmental disorder (PDD)/autism spectrum disorder (ASD). PDD-NOS is one of three forms of Autism Spectrum Disorders (ASD). PDD-NOS is often referred to as atypical autism.

Specific or Explicit diagnostic criteria for each of these disorders can be found in the Diagnostic & Statistical Manual of Mental Disorders (DSMIV-TR) as distributed by the American Psychiatric Association (APA).

Pathophysiology of Autism

Autism affects the amygdala, cerebellum, and many other unrecognized parts of the brain involved (Schumann C M, Nordahl C W. Neuroanatomy of autism. Trends Neurosci. 2008; 31(3):137-45. doi:10.1016/j.tins.2007.12.005. PMID 18258309). Autism does not have a clear unifying mechanism at either the molecular, cellular, or systems level; it is not known whether autism is a few disorders caused by mutations or changes converging on a few common molecular pathways, or is a large set of disorders with diverse mechanisms (like intellectual disability). Autism is said to be due to developmental factors that affect most functional brain systems (from Wikipedia on Autism).

Neuroanatomical studies and the associations with teratogens suggest that autism's mechanism includes alteration of brain development soon after conception (Frith U, Frith C D. Development and neurophysiology of mentalizing [PDF]. Philos Trans R Soc Lond B Biol Sci. 2003; 358(1431):459-73. doi:10.1098/rstb.2002.1218. PMID 12689373. PMC 1693139). This anomaly appears to start a cascade of pathological events in the brain that are significantly influenced by environmental factors. Just after birth, the brains of autistic children tend to grow faster than usual, followed by normal or relatively slower growth in childhood. It is not known whether early overgrowth occurs in all autistic children. It seems to be most prominent in brain areas underlying the development of higher cognitive specialization. Hypotheses for the cellular and molecular bases of pathological early overgrowth include the following:
1. An excess of neurons that causes local over connectivity in key brain regions (Casanova M F. The neuropathology of autism. Brain Pathol. 2007; 17(4):422-33)
2. Disturbed embryological neuronal migration during early gestation. Unbalanced excitatory-inhibitory networks (Schmitz C, Rezaie P. The neuropathology of autism: where do we stand? Neuropathol Appl Neurobiol. 2008; 34(1):4-11).
3. Abnormal formation of synapses and dendritic spines, (Schmitz IBID) for example, by modulation of the neurexin-neuroligin cell-adhesion system, or by poorly regulated synthesis of synaptic proteins. Disrupted synaptic development may also contribute to epilepsy, which may explain why the two conditions are associated.

FIG. 21 shows various regions of the cerebral cortex that could be involved in ASD signs and symptoms production including and besides prefrontal cortex. According to latest Courchesne, et al. postmortem study (Eric Courchesne, et al. Neuron Number and Size in Prefrontal Cortex of Children With Autism. JAMA. 2011; 306(18):2001-2010) of 7 autism and 6 control males brain showed that the brains of autism patients showed:
a) The prefrontal cortex had 67% percent more neurons compared to the normal brains;
b) The weight of the brain was more than the control children.
c) Previous studies has shown that the Macrocephaly occurs in 20% of individuals with autism on average and is usually due to megalencephaly—abnormal enlargement of the brain during childhood.
d) The enlargement is rarely present at birth; it develops during early childhood when head growth accelerates during the first 18 months of life.
e) Mean total brain, lobar, white matter, and gray matter volumes, including volume of the cortex, are significantly increased by 2 to 3 years of age in children with autism when compared with typically developing and also non autistic developmentally delayed individuals.
f) This prefrontal area of the cerebral cortex that is found having increased number of neurons is concerned with the language and communication which are some of the important signs and symptoms of autism originate (see FIG. 21 and see list below);
g) This abnormal development is said to begin during intrauterine life, the etiology being not known;
h) This finding further sets the vaccine and autism theory to rest or in the back burner;
i) The most important part of the finding is to start the treatment very early (below the age of 2) and do not wait for full blown case-hardened symptoms to develop.

These findings of the autopsy autism patients' brain are significant, because prefrontal cortex is involved in functions such as:
I. attention span
II. perseverance
III. planning and organization
IV. judgment
V. self-monitoring and supervision
VI. problem solving and internal supervision
VII. critical and forward thinking
VIII. learning from experience and mistakes
IX. impulse control, ability to feel and express emotions
X. influences on the limbic system involved in emotions
XI. understanding, empathy, compassion

The above functions mediated by the prefrontal cortex are the ones that show up in autism. It is important to note that one of areas of the brain that begins grows again and change just before puberty is the prefrontal cortex. That is why the teenagers can reason better, develop more control over impulses and make judgments better as the prefrontal cortex matures. Scientists call this part of the brain “the area of sober second thought.” Problems associated with dysfunction of the prefrontal cortex are due to developmental disorder or otherwise; as seen in autism are numerous. Factors that normally organize the brain as noted above appear to be disrupted due to changes in the neuropil mass. They are classically presented as signs and symptoms of autism patients also. They are:
I. short attention, concentration, interest span,
II. Easily distract acted
III. lack of perseverance, resolve, and insistence
IV. impulse control problems
V. hyperactivity
VI. chronic lateness, poor time management
VII. poor organization and planning
VIII. procrastination
IX. Lack of or unavailability of emotions, sentiment, feeling, and passion
X. misperceptions and poor judgments

XI. trouble learning from experience
XII. short term memory problems
XIII. social and test anxiety
XIV. Twisting the facts and/or lying

Interactions between the immune system and the nervous system begin early during the embryo, and successful neurodevelopment depends on a balanced immune response. It is possible that aberrant immune activity during critical periods of neurodevelopment is part of the mechanism of some forms of ASD. Although some abnormalities in the immune system have been found in specific subgroups of autistic individuals, it is not known whether these abnormalities are relevant to or secondary to autism's disease processes. As autoantibodies are found in conditions other than ASD, hence, the relationship between immune disturbances and autism remains controversial.

The relationship of neurochemicals to autism is not well understood; several have been investigated, with the most evidence for the role of serotonin and of genetic differences in its transport (Levy S E, Mandell D S, and Schultz R T. Autism. Lancet. 2009; 374(9701):1627-38). Others have pointed to a role for group I metabotropic glutamate receptors (mGluR) in the pathogenesis of one type of autism, Fragile X. Some data suggest an increase in several growth hormones; other data argue for diminished growth factors. Also, some inborn errors of metabolism are associated with autism but probably account for less than 5% of cases.

The mirror neuron system (MNS) theory of autism hypothesizes that distortion in the development of the MNS interferes with imitation and leads to autism's core features of social impairment and communication difficulties. The MNS operates when an animal performs an action or observes another animal perform the same action. The MNS may contribute to an individual's understanding of other people by enabling the modeling of their behavior via embodied simulation of their actions, intentions, and emotions (Williams J H G. Self-other relations in social development and autism: multiple roles for mirror neurons and other brain bases. Autism Res. 2008; 1(2):73-90. doi:10.1002/aur.15. PMID 19360654). Several studies have tested this hypothesis by demonstrating structural abnormalities in MNS regions of individuals with ASD, delay in the activation in the core circuit for imitation in individuals with Asperger's syndrome, and a correlation between reduced MNS activity and severity of the syndrome in children with ASD. However, individuals with autism also have abnormal brain activation in many circuits outside the MNS and the MNS theory does not explain the normal performance of autistic children on imitation tasks that involve a goal or object.

Autistic individuals tend to use different areas of the brain for a movement task compared to a control group (Powell K. Opening a window to the autistic brain. PLoS Biol. 2004; 2(8):E267). ASD-related patterns of low function and aberrant activation in the brain differ depending on whether the brain is doing social or nonsocial tasks. In autism, there is evidence for reduced functional connectivity of the default network, a large-scale brain network involved in social and emotional processing with intact connectivity of the task-positive network, used in sustained attention and goal-directed thinking. In people with autism, the two networks are not negatively correlated in time, suggesting an imbalance in toggling between the two networks, possibly reflecting a disturbance of self-referential thought. A 2008 brain-imaging study found a specific pattern of signals in the cingulate cortex which differs in individuals with ASD (Chiu P H, Kayali M A, Kishida K T et al. Self responses along cingulate cortex reveal quantitative neural phenotype for high-functioning autism. Neuron. 2008; 57(3):463-73).

The under connectivity theory of autism hypothesizes, that the autism is marked by under connectivity of high-level neural connections and synchronization, along with an excess of low-level processes (Just M A, Cherkassky V L, Keller T A, Kana R K, Minshew N J. Functional and anatomical cortical under connectivity in autism: evidence from an FMRI study of an executive function task and corpus callosum morphometry. Cereb Cortex. 2007; 17(4):951-61). Evidence for this theory has been found in functional neuroimaging studies on autistic individuals and by a brainwave study that suggested that adults with ASD have local over connectivity in the cortex and weak functional connections between the frontal lobe and the rest of the cortex (Murias M, Webb S J, Greenson J, Dawson G. Resting state cortical connectivity reflected in EEG coherence in individuals with autism. Biol Psychiatry. 2007; 62(3):270-3). The pathology in the frontal lobe its disrupted or affected connections due any kind of damage may play a major role in ASD. Damage to frontal lobes can also impair the executive function, that is the ability to plan, initiate, organize, carry out, monitor, and correct one's own behavior (W. W. Beatty and N. Monson, “Problem Solving By Patients With Multiple Sclerosis, “journal of the International Neurological Society 2:134-140, 1996; V. Goel and P. Grafman, “Are Frontal Lobes Involved With Planning Functions? Interpreting; Data from the Tower of Hanoi”, Neuropsychologia; 5:623-642, 1995). Other evidence suggests the under connectivity is mainly within each hemisphere of the cortex and that autism is a disorder of the association cortex (Minshew N J, Williams D L. The new neurobiology of autism: cortex, connectivity, and neuronal organization. Arch Neurol. 2007; 64(7):945-50).

From studies based on event-related potentials, transient changes to the brain's electrical activity in response to stimuli, there is considerable evidence for differences in autistic individuals with respect to attention, orientation to auditory and visual stimuli, novelty detection, language and face processing, and information storage; several studies have found a preference for non-social stimuli. For example, magneto encephalography (MEG) studies have found evidence in autistic children of delayed responses in the brain's processing of auditory signals. Our invention of the use of insulin and other therapeutic agents through the ORE and external ear will correct this neurological deficit in ASD; thus, cure or curtail the signs and symptoms of this condition. The olfactory neuroepthelium is the only area of the body in which an extension of the central nervous system comes into direct contact with the environment thus delivery of therapeutic agents to treat ASD directly to the brain is facilitated as described in our invention overcoming the blood brain barrier (BBB).

In recent years, the understanding of autism has grown enormously. Unfortunately, the general public, professionals in the medical, educational, and vocational fields, remain unaware of the effects of the disability on the family at home and work. Paradoxically, autistic afflicted people may exhibit both positive and negative responses to their environment such as: may make eye contact, show affection, smile and laugh, and demonstrate a variety of other emotions, although in varying degrees. The autistic children and adults can exhibit any combination of the behaviors in any degree of severity. Two individuals, both with the same diagnosis, may have varying skills and display very different actions, and each has a unique personality and combination of characteristics including aggressive and/or self-injurious behavior. These can include speech and voice characteristics, such as uninflected and robot-like or sing-song or echolalic speech. Stereotyped speech refers to a highly repetitive, specific language that is often centered on inappropriate and arbitrary topics. The failure to develop the ability to produce novel utterances (generative language), and the inability to produce normal into national patterns or to understand conversational speech are primary deficits present in children with ASD.

According to the DSM-IV or Diagnosis and Statistical Manual for Mental Disorders, 4th edition, published by the American Psychiatric Association (American Psychiatric Association, DSM IV, 2000), autism is classified as a pervasive developmental disorder (PDD) characterized by twelve diagnostic criteria. These criteria fall into three categories:

I. impairments in social interaction,

II. impairments in communication, and

III. a restricted repertoire of activities and interests.

Accordingly, a diagnosis of autism requires that a child display at least six of these twelve symptoms, with a minimum number in each category.

DSM IV Diagnostic Criteria for Autism. Diagnosis Criteria for 299.00 Autistic Disorder

DSM-IV-TRT™ DIAGNOSTIC CRITERIA FOR AUTISM (taken directly from the DSM-IV-TR page 75 (2000). American Psychiatric Association. (2000). Diagnostic and Statistical Manual of Mental Disorders, 4th ed. DSM-IV-TR. Washington D.C.: American Psychiatric Association).

A. A total of six (or more) items from (1), (2), and (3) with at least two from (1), and one each from (2) and (3):
(1) Qualitative impairment in social interaction, as manifested by at least two of the following:
I. marked impairment in the use of multiple nonverbal behaviors such as eye-to-eye gaze, facial expression, body postures, and gestures to regulate social interaction
II. failure to develop peer relationships appropriate to developmental level
III. a lack of spontaneous seeking to share enjoyment, interests, or achievement with other people (e.g., by a lack of showing, bringing, or pointing out objects of interest)
IV. lack of social or emotional reciprocity
   (2) Qualitative impairments in communication as manifested by at least one of the following:
I. delay in, or total lack of, the development of spoken language (not accompanied by an attempt to compensate through alternative modes of communication such as gesture or mime)
II. in individuals with adequate speech, marked impairment in the ability to initiate or sustain a conversation with others
III. stereotyped and repetitive use of language or idiosyncratic language
IV. lack of varied, spontaneous make-believe play or social imitative play appropriate to developmental level
   (3) Restricted repetitive and stereotyped patterns of behavior, interests, and activities, as manifested by at least one of the following:
I. encompassing preoccupation with one or more stereotyped and restricted patterns of interest that is abnormal either in intensity or focus
II. apparently inflexible adherence to specific, nonfunctional routines or rituals
III. stereotyped and repetitive motor mannerisms (e.g., hand or finger flapping or twisting, or complex whole-body movements)
IV. persistent preoccupation with parts of objects
   B. Delays or abnormal functioning in at least one of the following areas, with onset prior to age 3 years: (1) social interaction, (2) language used in social communication, or (3) symbolic or imaginative play.
   C. The disturbance is not better accounted for by Rett's Disorder or Childhood Disintegrative Disorder.

If a child does not fit the definition of autism given above, he or she may be diagnosed with a condition called Pervasive Developmental Disorder Not Otherwise Specified (PDD-NOS). Such a diagnosis of non-specific forms of PDD may include atypical types of autism that do not fall into the above categories because of late age of onset, for example, or sub-threshold or atypical symptoms. According to the DSMIV, this diagnosis is to be used when autistic-like behaviors are present—in particular, when there is severe impairment in the development of social and verbal communication skills—but, the child does not meet the criteria for classic autism or any other specific Pervasive Developmental Disorder, Schizophrenia, Schizotypal Personality Disorder or Avoidant Personality Disorder (American Psychiatric Association-ASA, DSM IV, 2000).

The following traits, as identified by the ASA, may also be present in persons with autism:
I. Insistence on sameness or resistance to change;
II. Difficulty in expressing needs; (i.e. uses gestures or pointing instead of words);
III. Repeating words or phrases in place of normal, responsive language;
IV. Laughing, crying, showing distress for reasons not apparent to others;
V. Prefers to be alone or aloof manner;
VI. Tantrums;
VII. Difficulty in mixing with others;
VIII. May not want to cuddle or be cuddled;
IX. Minor or no eye contact;
X. Unresponsive to normal teaching methods;
XI. Sustained odd play; Spins objects; Inappropriate attachments to objects;
XII. Apparent over-sensitivity or under-sensitivity to pain;
XIII. No real fears of danger;
XIV. Noticeable physical over-activity or extreme under-activity;
XV. Uneven gross/fine motor skills; and/or Not responsive to verbal cues (i.e. acts as if deaf although hearing tests in normal range).

As ASD being complex diseases, genetic and environmental factors including infections, toxic chemicals, vaccinations with preservative mercury, xenobiotics, dietary proteins and peptides, and a host of unsubstantiated unproven theories are postulated as to its etiology. Based on some studies autism is blamed on: infectious agent; infectious agents and response to vaccinations; heavy metals and other toxic chemicals; neuro immune abnormalities induced by xenobiotics and metals; autoimmune reaction induced by heavy metals; neuro immune antibodies induced by dietary proteins and infectious agents; food allergies and intolerance to glutein; as well as neurotransmitters and neuro immune miscommunication. There are indications that basic defect appears to be a decrease in CNS Serotonin activity despite elevated free tryptophan levels in the serum in autism. Abnormal serotonin metabolites seen in autistic children may significantly contribute to their mental dysfunction (Warren R. P., Singh V, K. (1996) Elevated serotonin levels in autism: association with the major histocompatibility complex. Neuropsychobiology. 34(2):72-75. Cook E. H., Leventhal B. L. (1996). The serotonin system in autism. Current Opinion in Pediatrics. 8(4):348-354. McDougle C. J., Naylor S. T., Cohen D. J., Aghajanian G. K., Heninger G. R., Price L. H. (1996) Effects of tryptophan depletion in drug-free adults with autistic disorder. Archives of General Psychiatry. 53(11):993-1000). Drugs such as LSD, psilocybin, ergot, and other hallucinogens are serotonin analogs, and a number of serotonin metabolites are known to be hallucinogens. It is also interesting to note that serotonin and its metabolites are produced in, and absorbed from, the intestines. Therefore, detection of high or low levels of serotonin along with antibodies to serotonin, somatostatin, vasoactive intestinal peptides, DPP IV, pro-dynorphin and dynorphin may indicate disturbance in gut-neuro-immune communication.

According to the ASA reports, autism occurs more frequently than expected among individuals who have certain medical conditions, including Fragile X syndrome, tuberous sclerosis, congenital rubella syndrome, and untreated phenylketonuria (PKU). A number of harmful substances ingested during pregnancy also have been linked with an increased risk of autism.

In the search for the causes of autism, virtually every area of the brain has been investigated and implicated. On the other hand, studies have shown that structures of the temporal lobe, the anterior temporal cortex, the anterior Cingulate cortex, and the limbic system (e.g., the hippocampus, corpus callosum, and thalamus) are most likely to be mainly responsible for the deficits of autism. These brain structures normally mediate the processing of emotional and social information, which are the principal characteristics that are disordered in autism.

Autistic individuals have difficulty interacting with their environment and are often excessively sensitive to external stimuli. For example, some autistic patients have described normal visual or auditory input as being perceived as amplified and overwhelming. That means, the autistic persons are hypersensitive to, and disturbed or bothered by, sensory input that non-autistic people would find to be normal. It is supposed that neural over activity or abnormal action within the brain of autistic patients may be in part responsible for such hypersensitivity.

Interestingly, postmortem examinations of autistic patient's brains show abnormally small, densely packed cells in many areas of the brain which suggests that normal developmental pruning of axons, dendrites, and synapses in the brain of an autistic patient has not occurred at the normal rate. Hence, many autistic patients have an excess number of neural connections within their brain which may contribute to excess neural activity in some regions of the brain, thereby resulting in abnormal sensitivity to external stimuli and, in some cases, enlarged brain areas. In support of this theory, studies have also shown that the right anterior temporal cortex, anterior cingulate cortex, and thalamus are overactive in many autistic patients. Over activation of these areas suggests that more sensory channels are activated in autistic patients in response to external stimuli than in non-autistic individuals. This sensory channel activation may be in part responsible for the hypersensitivity to external stimuli exhibited by many autistic patients.

Studies have shown that the right sides of the thalamus, hippocampus, and peri corpus callosal areas are hyper perfused compared to the left sides within some autistic patients. Such hyper perfusion suggests that some autistic individuals may experience neural over activity especially within the right side of the brain. Hence, use our invention on the left side of the nose to begin with to activate and compensate for the right side hyperactivity.

Hence, it is believed that applying an appropriate stimulus to selectively reduce or interrupt some brain activity in one or more areas within the brain may be useful in treating autism. The stimulus may be configured to decrease neural activity within the brain of autistic patients, thereby ameliorating or eliminating an autistic patient's hypersensitivity to external stimuli. Consequently, the delivery of a stimulus to sites within the brain to treat the autism could be in the form of electrical stimulation current, one or more drugs, gene infusion, chemical stimulation, thermal stimulation, electromagnetic stimulation, mechanical stimulation, and/or any other suitable stimulation.

It is believed that an autistic patient experiences neural over activity, especially within the right side of his or her brain. Hence, in some examples, the stimulus may be applied to a stimulation site located within the right side or hemisphere of the brain to decrease neural activity therein or to provide other beneficial effects to treat autism.

The MRI and other scan studies have not been definite indicators of autism and are inconclusive. Two MRI studies of total brain and volume found increased total brain volume above the lower boundary of the brainstem, reflecting increased tissue volume and a follow-up study reported that the enlargement of the cerebral hemisphere was regional, involving occipital, parietal, and temporal regions. A series of MRI studies focusing on the cerebellar vermis revealed decrease in the mid-sagittal area of vermal lobules. The dissociation between the sizes of the cerebral cortex and corpus callosum was interpreted as evidence of abnormal development of neural connectivity between the hemispheres. Our invention may help to correct this dysfunction.

In summary, the etiology of autism is poorly defined both at the cellular, molecular and developmental levels. Based on the fact that the seizure activity is frequently associated with autism and that abnormal evoked potentials have been observed in autistic individuals in response to tasks that require attention, some investigators have recently proposed that autism might be caused by an imbalance between excitation and inhibition in key neural systems including the cerebral cortex.

One needs to know for sure that the autism is not caused by bad parenting. No known psychological factors have been found and autism is not a mental illness. Vaccination theory with mercury preservative (Thimerosal) still rages without any clear cut answer, although all the evidence indicates, it may not be factor in development of autism. Children with autism and PDD are either born with the disorder or with the risk or possibility to develop it. One needs to understand, that the autistic children are not unruly kids who choose not to behave.

So far, there are no ultimate definitive diagnostic tests for autism. It remains one of the neurological disorders that have to be diagnosed almost entirely through behavioral symptoms. All we know is that autism interferes with the normal development of the brain in the areas of reasoning, social interaction, communication skills and emotions such as love and empathy. Children and adults with autism characteristically have deficiencies in verbal and nonverbal communication, social interactions, and leisure or play activities. Autistic people may exhibit repeated body movements such as hand flapping, rocking, or spinning; they may have unusual responses to people or attachments to objects; and they may resist changes in routines. A number of cases may display aggressive or self-injurious behavior.

The Past and the Present Methods of Autism Treatments

The treatments for autism has been too often been filled with false hope. Therapies for treatment of autism include conventional, intensive Applied Behavioral Analysis (ABA) therapy as well as a host of alternative approaches, including a gluten-free and casein-free (GFCF) diet, hyperbaric oxygen chambers, chelation, aroma therapies, electro-magnetics, spoons rubbed on his body, multivitamin therapy, B-12 shots and a range of prescription psychosomatic drugs. There was the gentleman who claimed he had cured his son by hugging him a lot—he wrote a best-selling book about it—and others who claimed they had cured their child by teaching him or her to swim. In addition, there was the Secretin hormone controversy, in which parents paid thousands of dollars for a hormone believed to successfully treat autism before several clinical trials showed no actual impact.

In spite of voluminous doses of research and the knowledge, there is no cure for autism. There are a number of therapeutic agents developed for other conditions, which have been found to be to some extent helpful in treating a limited number of the symptoms and behavioral problems such as hyperactivity, impulsivity, attention difficulties, and anxiety. Examples of therapeutic agents used to treat symptoms associated with autism include: Serotonin re-uptake inhibitors (e.g. clomipramine (Anafranil), fluvoxamine (Luvox) and fluoxetine (Prozac)) which have been effective in treating depression, obsessive-compulsive behaviors, and anxiety that are sometimes present in autism. Studies show that they reduce the frequency and intensity of repetitive behaviors, decrease irritability, tantrums and aggressive behavior, improvements in eye contact and responsiveness. Other drugs, such as Elavil, Wellbutrin, Valium, Ativan and Xanax, are also being tried to decrease the behavioral symptoms.

The extensively studied psychopharmacologic agents in ASD have been anti-psychotic medications developed for treating schizophrenia. These therapeutic agents do decrease hyperactivity, stereotypic behaviors, withdrawal and aggression in autistic children. Four anti-psychotic medications that have been approved by the FDA are clozapine (Clozaril), risperidone (Risperdal), olanzapine (Zyprexa) and quetiapine (Seroquel). However, only risperidone has been investigated in a controlled study of adults with autism. Stimulants, such as Ritalin, Adderall, and Dexedine, used to treat hyperactivity in children with ADHD have also been prescribed for children with autism. They are said to increase focus, and decrease impulsivity and hyperactivity in autism; regrettably, adverse behavioral side effects are often observed.

Studies (US 2009/0048348 AI) show that administering an effective doses of a NMDA-receptor antagonist or a pharmaceutically acceptable salt thereof improve frontal executive functions associated with autistic symptoms, including, but not limited to, speech expression and decreased perseveration without any side effects associated.

Currently, autism spectrum disorders are treated using: applied behavior analysis or other behavior modification techniques; dietary alteration such as a gluten or casein free diet; large doses of vitamin B6 in combined with magnesium; medications specific symptoms such as anxiety and depression and include agents such as fiuoxetine, fiuvoxamine, sertraline and clomipramine; and, antipsychotic medications such as chlorpromazine, thioridazine, and haloperidol have been used to treat behavioral problems. Anticonvulsants such as arbamazepine, lamotrigine, topiramate, and valproic acid have been given to prevent seizures.

Regrettably, the current treatments for autism spectrum and related disorders are mainly symptomatic. They have proven futile in allowing such children and adults to become symptom free, or disorder free. Therefore, there is an unmet need in the art for alternative treatments for autism spectrum disorders and related pathologies. So far, none of the therapies had uniform success; only an improvement some if not all functions. Our method of treatment will attack the problems and will bring relief to thousands ASD patients.

### OBJECTIVES OF THE PRESENT INVENTION

It is the object of the present invention to provide methods and apparatus for delivery of insulin, IGF-1, and other adjuvant therapeutic compounds—neurologic agents to the brain by passing the BBB through the intranasal olfactory region (ORE) for the treatment of ASD.

A further objective and goal of this invention is to develop a means of selective delivery of a neurologic agent to the areas of the brain which are affected in the ASD and other related brain disorder.

Still, another objective of this invention is to develop a composition that can cause absorption of the neurologic agent into olfactory neurons and along the olfactory neural pathway to neurons in the brain afflicted in ASD and related CNS disorders.

Another goal of this invention is to provide prophylactic treatment for prodromal symptoms akin to ASD evolving after vaccination or similar inciting provocative event.

Another goal of this invention is to provide prophylactic treatment for neurodegenerative diseases associated with ASD and to treat and/or prevent associated loss of function.

It is the aim of the present invention to provide methods and apparatus as can be employed to deliver such compounds through the ORE, a minimally invasive approach to treat ASD.

It is the goal of the present invention to provide methods and apparatus that can facilitate delivery of large molecular weight compounds through the ORE to treat ASD.

It is the object intention of the present invention to provide cost-effective methods for delivery of compounds intranasally to ORE in the treatment of ASD.

It is still a further purpose of the present invention to provide improved methods for remedying or modifying neurological activities and disorders via delivery of therapeutic agents and compounds with insulin intranasally through ORE to treat ASD.

It is an additional object of the present invention to provide improved therapeutic agents such as insulin and IGF-1; and their methods of delivery for treating ASD through external ear and ORE which upon reaching the neuropil of the cortex and brain stem to relive the symptoms of ASD.

It is still an additional object of the present invention to provide improved methods for delivery if therapeutic agents for treating neurological diseases (for example, Alzheimer's, Parkinson's, depression, senile dementia, ALS, MS, etc.), associated with ASD.

It is also to be appreciated that the present object of this invention is meant to include substantially any safe therapeutic agents along with insulin and/or IGF-1 delivered to the designated neuropile and neuronal nuclei through the intranasal ORE and external auditory meatus.

It is further object of this invention to be valued that this method of treatment of ASD described herein by way of illustration and not limitation, and that the range of the present invention includes other possibilities which would be obvious to someone of ordinary skill in the art who has read the present patent application.

It is the object of this invention additionally to be appreciated that, whereas, preferred embodiments of the present invention are described with respect to application of the therapeutic agents to be understood in the context of the present patent application and in the claims as being substantially equivalent to supplying the therapeutic agents directly to neuropile similar to delivery of therapeutic agents across breached BBB.

### SUMMARY OF THE INVENTION

The present invention consists of a method of treating autistic spectrum disorder (ASD) in a patient; the method comprising administering to the patient an effective dose of insulin intranasally specifically to the olfactory region (ORE).

The present invention consists of a method of treating autistic spectrum disorder (ASD) in a patient; the method comprising administering to the patient an effective dose of Insulin-like growth factor-I (IGF-1) intra nasally to olfactory region (ORE).

The present invention includes a method of treating an autistic spectrum disorder (ASD) in a patient; the method comprising administering to the patient an effective dose of insulin through the external ear.

The present invention consists of a method of treating autistic spectrum disorder (ASD) in a patient; the method comprising administering to the patient an effective dose of Insulin-like growth factor through the external auditory meatus.

In yet another aspect of the present invention, the antibody is selected from the group consisting of a polyclonal antibody, a monoclonal antibody, a humanized antibody, and a biologically active fragment of an antibody, being administered intranasally to ORE and to external auditory meatus with insulin and IGF-1 to counter the autoantibodies in the CNS of ASD patients.

The present invention contains a method of treating an autistic spectrum disorder in a patient, the method comprising administering to the patient an effective dose of an antibody to tumor necrosis factor alpha (TNF alpha) with insulin intranasally to ORE.

In still another aspect of the present invention, additional aspects of this disclosure to treat ASD, combinatorial formulations and methods are provided comprising insulin with an effective dose of oxytocin or an oxytocin analog including carbetocin in combination with one or more secondary adjuvant agent(s).

Yet, still in another aspect of the present invention, additional adjuvant therapies to treat ASD may also include intranasal insulin with glucose.

In still another aspect of the present invention, additional adjuvant therapies to treat ASD may also include intranasal insulin with Etanercept.

In still another aspect of the present invention, additional adjuvant therapies to treat ASD may also include intranasal insulin with progesterone.

In still another aspect of the present invention, additional adjuvant therapies to treat ASD may also include intranasal insulin with sibutramine hydrochloride.

In still another aspect of the present invention, additional adjuvant therapies to treat ASD may also include intranasal insulin with tricyclic antidepressant or the selective serotonin reuptake inhibitors.

In still another aspect of the present invention, additional adjuvant therapies to treat ASD may also include intranasal insulin with leuprolide.

In still another aspect of the present invention, additional adjuvant therapies to treat ASD may also include intranasal insulin with 5HT-3 receptor blockers.

The present invention consists of a method of treating autistic spectrum disorder (ASD) in a patient, the method comprised of administering to the patient an effective dose of insulin intranasally specifically to the olfactory region with Naltrexone.

The present invention consists of a method of treating autistic spectrum disorder (ASD) in a patient, the method comprised of administering to the patient an effective dose of insulin intranasally specifically to the olfactory region with additional adjuvant therapeutic agent Vitamin B₁, B₆, B₁₂ and D₃.

The present invention consists of a method of treating ASD in a patient, the method comprised of administering to the patient an effective dose of insulin intranasally specifically to the olfactory region with additional adjuvant therapeutic agent magnesium sulfate.

The present invention consists of a method of treating autistic spectrum disorder in a patient, the method comprised of administering to the patient an effective dose of insulin intranasally specifically to the olfactory region with additional adjuvant therapeutic agent topiramate.

The present invention consists of a method of treating autistic spectrum disorder in a patient, the method comprised of administering to the patient an effective dose of insulin intranasally specifically to the olfactory region with additional adjuvant therapeutic agent memantine.

The present invention consists of a method of treating autistic spectrum disorder in a patient, the method comprised of administering to the patient an effective dose of insulin intranasally specifically to the olfactory region with additional adjuvant therapeutic agent haloperidol.

The present invention consist of a method of treating autistic spectrum disorder in a patient, the method comprised of administering to the patient an effective dose of insulin intranasally specifically to the olfactory region with additional adjuvant therapeutic agent Celecoxib.

The present invention consist of a method of treating autistic spectrum disorder (ASD) in a patient, the method comprised of administering to the patient an effective dose of insulin intranasally specifically to the olfactory region with additional adjuvant therapeutic agent ACE inhibitor.

In still another aspect of the present invention, additional adjuvant therapies may also include intranasal insulin with behavioral modification and changes in diet such as a gluten-casein free diet, transfer factors and probiotics.

Nevertheless, another aspect of the present method of the invention employs transneuronal antegrade and retrograde transport of the neurologic therapeutic agents entering through the olfactory system of the brain reach through the interconnected areas of the brain such as the hippocampal formation, amygdaloid nuclei, and nucleus basalis of Meynert, locus coeruleus, the brainstem raphe nuclei and other neurological structures in front of the brain stem.

In still another aspect of the present invention, additional adjuvant therapeutic agent is the antibody administered to counter effect of the cytokines generated post vaccination or otherwise by the route selected from the group consisting of intramuscularly, intrathecally, intravenously, intradermally, subcutaneously, cutaneously, iontophoretically, topically, locally, and intranasally (ORE).

Another aspect of the present invention includes; a kit for treating an autism spectrum disorder in a patient, the kit comprising insulin, insulin-like growth factor-I (IGF-1), selected suitable adjuvant therapeutic agents administered, orally. The kit further carries therapeutic agents selected to be administered to the intranasally to ORE and the ear to treat ASD. Further, the kit is comprised of an applicator to the ear, nose dropper and delivery catheter with balloon with syringes, antiseptics wipers, alcohol gadgets, neck support to be used intranasally to deliver therapeutic agents of this invention to ORE and the ear to deliver with an instructional material for the use thereof.

### BRIEF DESCRIPTION OF THE DRAWINGS

The purpose of the present invention will become readily valued and understood from deliberation of the following comprehensive descriptions of the preferred embodiments when taken together with the accompanying drawings in which:

FIG. 1 is the diagrammatic presentation 100 of the olfactory mucosa covering the medial and lateral walls of the nose.

FIG. 1a is the diagrammatic presentation 100a showing vestibule, respiratory and olfactory mucosa of the lateral and medial walls of the nose.

FIG. 2 is the diagrammatic presentation of the lateral wall 200 of the nerve structures in the nose.

FIG. 3 is the diagrammatic presentation of the medial wall 300 of the nerve structures in the nose.

FIG. 4 views of diagrams 400 showing histology of the olfactory mucosa and the route of transmission of the insulin and other therapeutic agents to the CNS used in this invention.

FIG. 5 is the modified electron micrograph of the olfactory nerve fasciculi 500 surrounded by Perineural epithelium and sub Perineural epithelial space around the axon bundles.

FIG. 6 is the modified electron micrograph of the olfactory nerve fasciculi 600 surrounded by Perineural epithelium and sub Perineural epithelial space around the axon bundles with therapeutic agents.

FIG. 7 is the drawing of the longitudinal section 700 of the olfactory bulb showing the histology and route of therapeutic agent's journey to CNS from ORE.

FIG. 8 is the diagrammatic presentation 800 of the invention to treat autism using special catheter device with a balloon at the tip to spray or drip drop by drop of the therapeutic agents.

FIG. 8a is the diagrammatic presentation 800a of the invention with special catheter device with a balloon at the tip in ORE position.

FIG. 9 is the diagrammatic presentation 900 of the invention to treat autism using catheter delivery system.

FIG. 10 is the drawing 1000 of the section of the olfactory mucosa showing the routes taken by the insulin and various therapeutic agents to CNS deposited at the olfactory region to treat autism in our invention.

FIG. 11 is the diagrammatic presentation 1100 of the inventive device to be used to deliver the therapeutic agents continuously through the nasal cavity to the olfactory regional nerves for long periods of time.

FIG. 12 is the drawing of the nerve fasciculi 1200 showing the structure of the peripheral nerve fasciculi, its coverings, Virchow-Robin space and blood vessels.

FIG. 13 Section of nerve fasciculi 1300 showing A, B, C and D enzyme activity in the Perineural epithelium cells with sub Perineural epithelial space which conducts therapeutic agents to CNS SAS.

FIG. 14 is the Histological diagram 1400 drawn after extensive light and electron microscopic study of the myelinated nerve axons within the nerve fasciculi and the site of entry of therapeutic agents into axons.

FIG. 15 is the drawing of the location of the circumventricular organs 1500 of the brain.

FIG. 16 is the diagram 1600 of the Virchow-Robin space in the central nervous system which plays a role in spread of insulin and therapeutic agents of our invention to treat autism.

FIG. 17 is the neuro anatomical drawing 1700 of the conduction of the auditory impulses from outside to be perceived as sound in the CNS.

FIG. 18 is the drawing of the external, middle and internal ear involved 1800 in conduction of sound and delivery of insulin and therapeutic agents to the tympanic plexus and internal ear.

FIG. 19 is the drawing of the ear 1900 with patient lying left lateral position for delivery of therapeutic agents through the ear to treat autism.

FIG. 20 is the drawing of the neuropil 2000 between the CSF of the central canal and subarachnoid space (SAS) which transmit the therapeutic agents of our invention to treat ASD.

FIG. 21 is the drawing of various regions of the cerebral cortex including prefrontal cortex 2001 involved in production of autism signs and symptoms.

### DETAILED DESCRIPTION OF THE INVENTION

### Description of the Terms Used in this Invention

As used in the specification and claims, the singular forms “a,” “an” and “the” include plural references unless the circumstance dictates otherwise. For example, the term “a cell” includes a plurality of cells, including mixtures thereof.

The terms “therapeutic,” “therapeutically effective doses,” and their cognates refer to that doses of a substance, e.g., of a protein, e.g., insulin, of an IGF-I, that results in prevention or delay of onset, or amelioration, of one or more symptoms of a disease.

The term “therapeutic agents” refers to drugs used to treat ASD and other associated diseases which include various known therapeutic agent, as well as other pharmaceutical, biochemical, nurticeuticals, and biological agents or compounds those results in prevention or delay of onset, or amelioration, of one or more symptoms of a disease.

As used herein, the term “treating” or “treatment” and “example” refers to both therapeutic treatment and prophylactic or preventative measures. Those in need of treatment include those already with the disorder as well as those prone to having the disorder or diagnosed with the disorder or those in which the disorder is to be prevented.

A “subject,” “individual” or “patient” is used interchangeably herein, which refers to a vertebrate, preferably a mammal, more preferably a human.

The term “mammal”(s)” include, but are not limited to, mice, rats, monkeys, humans, farm animals, sport animals, and pets.

As used herein the term “ameliorate” is synonymous with “alleviate” or “relief” or “relieve” means to reduce or ease. For example, one may ameliorate the symptoms of a disease or disorder by making them more bearable or completely cured of the disease.

The term “neuropile (neuropil)” in the following description refers to an intricate, complex network of unmyelinated axons, dendrites, and glial branches that form the bulk of the central nervous system's grey matter with Microglial cells with BV endowed with BBB and in which nerve cell bodies are embedded.

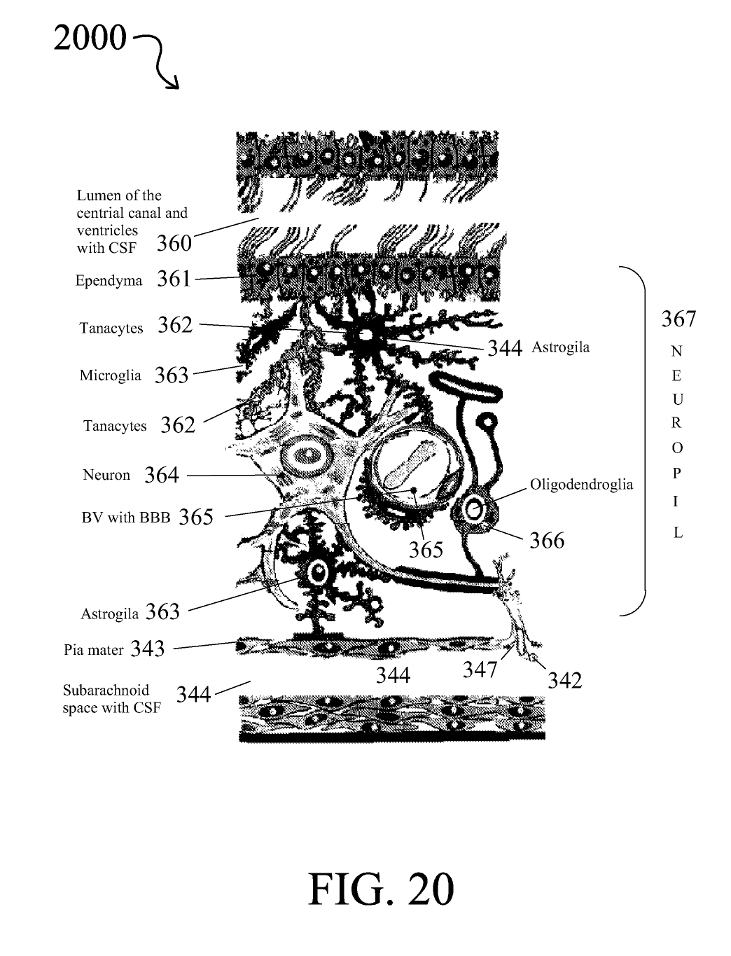

The term “BBB” (blood brain barrier) refers to the 400 miles of blood vessels in the form of capillaries that supplies the neuropil and forms the bulk of the blood supply (20% of the cardiac output) of the central nervous system's gray matter in which the nerve cell bodies lay surrounded and embedded in the neuropil (see FIG. 20). The white matter is mostly composed of axons and glial cells that is, generally, not considered to be a part of the neuropile. The BBB is formed by a complex cellular system of non-fenestrated endothelial cells, astroglia, pericytes, perivascular macrophages, and an amorphous non-cellular basal lamina and missing smooth muscle cells. Compared to other tissues, brain endothelia have the most intimate cell-to-cell connections: endothelial cells adhere strongly to each other, forming structures specific to the CNS called “tight junctions” or zonula occludens which prevent cell migration or cell movement between endothelial cells. Astrocytes end feet, cover these brain capillaries, build a continuous sleeve and maintain the integrity of the BBB by the secretion of soluble growth factors (e.g., gamma-glutamyl transpeptidase) necessary for the endothelial cells to develop their BBB feature. The “blood-brain barrier” (BBB) presents a major problem in the administration of therapeutic agents and neurotrophin so as it prevents a sufficient concentration of these potential therapeutic agents from reaching the target areas of the human brain to cure or curtail ASD. Fortunately, the ORE proves a route evading the BBB, presenting the select therapeutic agents directly to the neuropil of the brain to the site of pathology to treat CNS diseases.

The term “olfactory region” “ORE” includes olfactory mucosa, sphenopalatine ganglion and its branches, branches from the trigeminal nerve, olfactory nerve fasciculi as they enter the olfactory bulb, the communicating blood vessels of this region to the CNS. It is located in the upper third of the medial and lateral wall of the nose and covers the entire roof of the nose (cribriform plate of the ethmoid bone).

The term “olfactory mucosa” “OM” referrers to the olfactory area in the upper part of the nose which contains olfactory receptor bipolar neurons within its other layers located in the upper part of the lateral and medial wall of the nose. Olfactory neuro-epithelium (OM) is the only area of the body in which an extension of CNS comes in contact with the external environment.

The term “tumor necrosis factors,” “TNF,” “cytokines” refers to a naturally occurring cytokines present in humans or mammals, which plays a key role in the inflammatory immune response and in the response to infection and it is found elevated in ASD, also. TNF are formed by the cleavage of a precursor transmembrane protein, forming soluble molecules which aggregate in vivo to form trimolecular complexes which bind to receptors found on a variety of cells. This binding produces an array of pro-inflammatory effects, including release of other pro-inflammatory cytokines, including IL-6, IL-8, and IL-I; release of matrix metalloproteinases; and up regulation of the expression of endothelial adhesion molecules, further amplifying the inflammatory and immune cascade by attracting leukocytes into extra vascular tissues.

The term “perineural epithelium” ‘PE” means it is a histological structure of continuous flat squamous cell layers completely surrounding the nerve fasciculi (axons bundles) and separating the axons from the tissue space around the nerve bundle and protecting them.

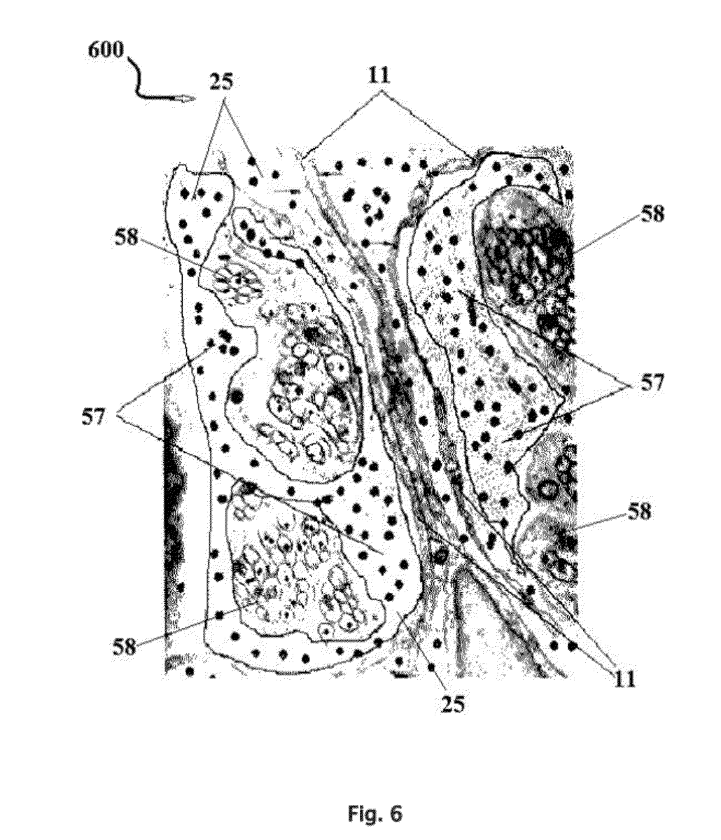

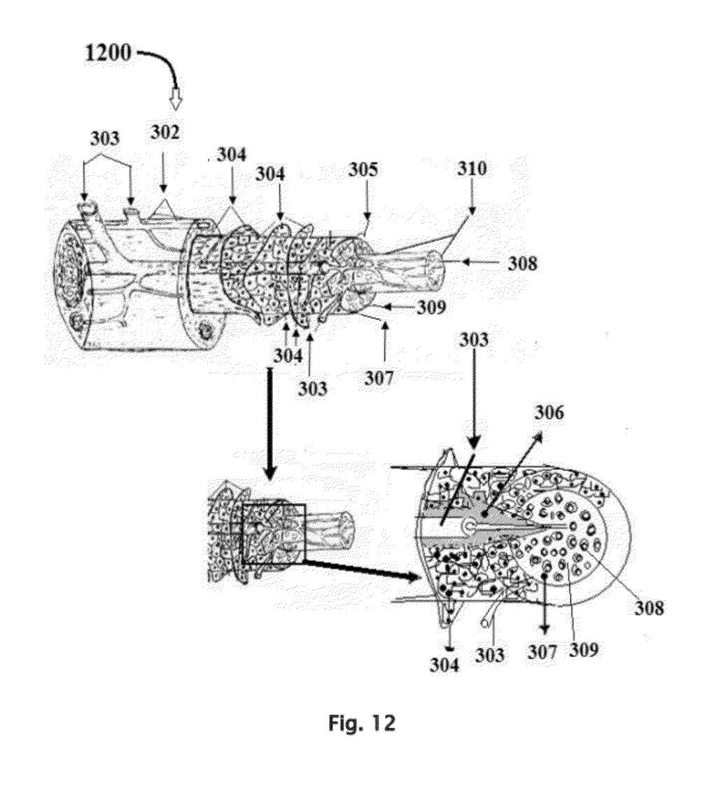

The term “sub perineural epithelial space” “sub PE” used to describe the tissue space between the nerve bundles of axons (fasciculi) and below the perineural epithelium (FIGS. 6, 12 13). The perineural epithelium is continuous with the pia and arachnoid mater of CNS and the sub perineural epithelium space is continuous with the subarachnoid space of the CNS including the spinal cord.

The term “biological or biologics” means the monoclonal antibodies, fusion proteins, and all of the specific molecules which act as TNF antagonists and interleukin antagonists in contrast to drugs that are chemically synthesized. For the purpose of this patent, a biologic is defined as a molecule produced through recombinant DNA technology which is derived from the DNA of a living source which may include humans, animals, or microorganisms. Cytokine antagonists are also a type of biologics.

The term “antibodies” “immunoglobulins” means the proteins produced by one class of lymphocytes (B cells) in response to specific exogenous foreign molecules (antigens, infections). They can be synthesized in the laboratory, also.

The term “monoclonal antibodies” “mAB”, means the identical immunoglobulins which recognize a single antigen that are derived from clones (identical copies) of a single line of B cell. Monoclonal antibodies with affinity for a specific cytokine reduce their biologic effect of a cytokine. Monoclonal antibodies (mAB) can be a cytokine blocker, or a cytokine inhibitor, or as a cytokine antagonist. The terms blocker, inhibitor, and antagonist are used interchangeably with respect mAB against cytokines.

The term autism and ASD are loosely and inter-changeably used.

Advantages of Olfactory Region Delivery of Therapeutic Agents in the Treatment of Autism in this Invention

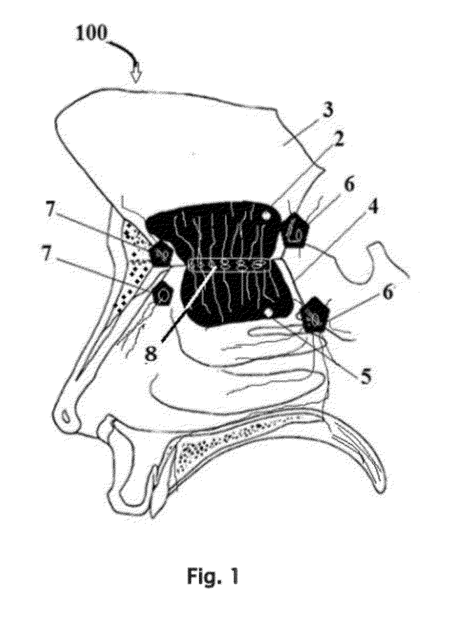

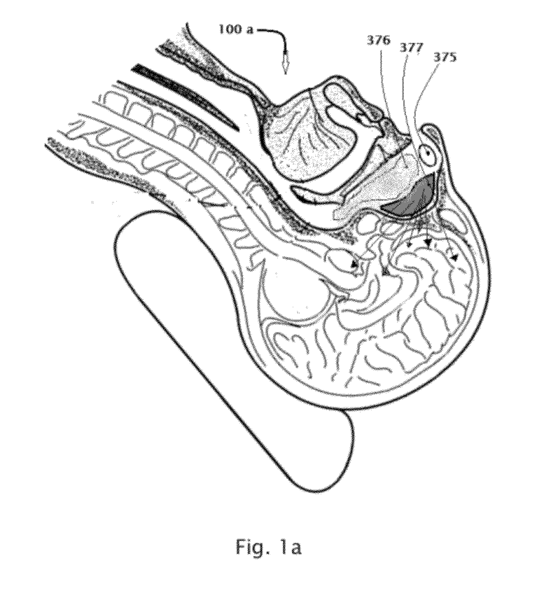

This present invention describes such a method of use of insulin, IGF-1 and other adjuvant therapeutic agents to treat ASD through delivery of the said drug through the olfactory region (ORE) to be transported to the CNS to cure and/or curtains ASD. Olfactory region administration of insulin, IGF-1 and various adjuvant pharmaceutical, biochemical, nurticeuticals, and biological agents or compounds developed or being developed to treat autism has many of the following advantages when compared to oral or systemic administration:
i. Due to the close proximity of the olfactory nerves, sphenopalatine ganglion and its branches, and trigeminal nerves to the central nervous system (FIGS. 1-6): CSF insulin and therapeutic agents used to treat autism concentrations exceed plasma concentrations, making this an important method of rapidly achieving adequate CSF drug concentrations for centrally acting medications to treat ASD.
ii. Ease and convenience: This method of ORE drug administration is essentially painless, does not require strict sterile technique, intravenous catheters or other invasive devices and it is immediately and readily available in all patients at all times.
iii. High therapeutic efficacy: Due to the achievement of higher local concentration in the CNS through SAS delivery where it is needed compared to the rest of the body due to presence of rich nerve plexus in the ORE.
iv. Increased efficacy of insulin and other therapeutic agents: Due to the ability of the administered therapeutic molecule is bioavailable to reach the target tissue without degradation caused by digestive enzymes, hepatic or systemic circulation (first phase metabolism); and the ability of the insulin to augment and amplify the effects of other therapeutic agents used to treat ASD.
v. Fast onset of action: Due to their proximity to the CNS, the site where they are needed; and most of the therapeutic agents reach the CNS within 30 minutes.
vi. Longer duration of action: Due to therapeutic agents localization in slow moving and exchanging (3 times a day) CSF.
vii. Possible fewer side effects, and less expensive: Due to lower dose of therapeutic agents required.
viii. Very much improved efficacy: Due to improved delivery of the therapeutic molecule to the CNS, the site of the disease.
ix. Another advantage of olfactory mucosal ORE delivery is: It does not require any modification of the therapeutic agents. A wide variety of therapeutics, including both small molecules and macromolecules, can be targeted from the olfactory region to the CNS system to treat ASD as well as any and all diseases of the CNS including Alzheimer's disease, stroke, depression, schizophrenia, Parkinson's, pain, addiction, PTSD, autism and other CNS disorders.
x. The advantage of intranasal olfactory regional delivery does not require any modification of the therapeutic agents and does not require that drugs be coupled with any carrier like in case of drug delivery across the Blood brain barrier (BBB). A wide variety of therapeutic agents, including both micro molecules and macromolecules can be successfully delivered intranasal olfactory region delivery method
xi. It is Low cost, patient and healthcare provider friendly, non-invasive, non injectable, and safe method when used appropriately

There are some Limitations to this mode of therapeutic agents use through ORE. They are:
I. Concentration achievable in different regions of the brain and spinal cord, varies with each agent, many medications are not adequately concentrated to achieve ideal dosing volumes
II. Delivery to CNS is predictably decrease with increasing molecular weight of drug,
III. Some therapeutic agents may cause irritation to the region and olfactory mucosa that can impact the absorption of therapeutic agents delivered to ORE.
IV. Nasal congestion due to cold or allergies may interfere with this method of delivery if they are used for local or systemic effects, and has no such effect on CNS delivery.
V. Recurrent everyday use of this ORE route can result in regional damage (e.g. infection, anosmia-loss of smell).
VI. Limited medications that can be delivered through ORE.

Detailed Description of the Diagrams Explaining the Invention to Treat Autism and how the Therapeutic Agents Reach the CNS to Cure or Curtail ASD

With reference now to the various figures in which identical embodiments are numbered alike throughout the description of the preferred therapeutic agents, examples, and the techniques of the present invention will now be presented below. These diagrams represent the present invention and describe how the insulin, IGF-1 and other adjuvant therapeutic agents to treat ASD reach the site of pathology in the CNS to cure/curtail ASD. While the preferred embodiment of the present invention has been described, it should be understood that various changes, adaptations and modifications may be made thereto. It should be understood, therefore, that the invention is not limited to details of the illustrated invention examples.

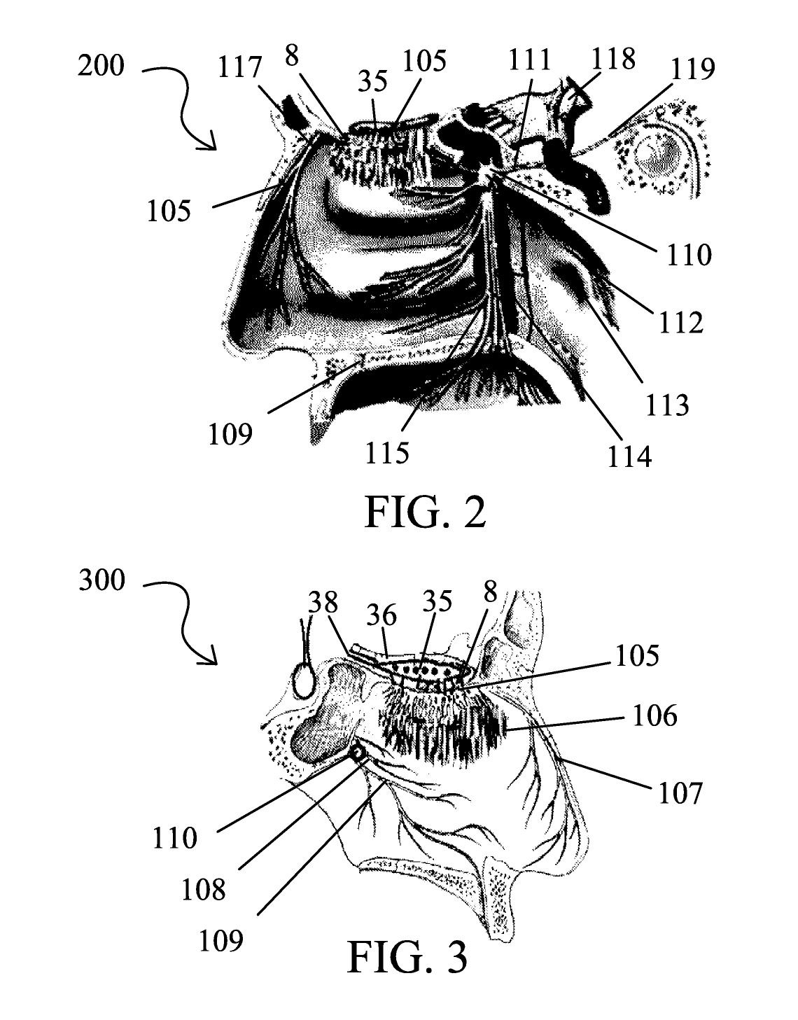

FIG. 1 is the diagram of the lateral and medial wall of the nasal cavity 100 reflected back at cribriform plate of the ethmoid bone; showing olfactory region (ORE) with various nerve structures (shown in black surface with white lines) that the insulin and the other anti-autism therapeutic agents come in contact and transported to the CNS retrograde to the brainstem, thalamic, hypothalamic, prefrontal and other cortical centers, cerebellum and other neuropil. Note the olfactory mucosa (OM) with olfactory receptor and its nerve fasciculi 2, 5 cover extensive areas of the medial 3 and lateral 4 wall of the upper part of the nasal cavity which is separate from the respiratory part of the nose and passé through the cribriform plate of the ethmoid bone 8 to olfactory bulb. This region contains the sphenopalatine ganglion (Pterygopalatine) 6 with its extensive central and peripheral connecting branches (see FIG. 2 below). This ORE also surrounded by anterior ethmoidal nerves 7 connected to trigeminal nerves. The therapeutic agents including insulin used to treat autism in this invention passed on to the CNS and the CSF through the sub perineural epithelial space to SAS through the olfactory nerves; trigeminal nerve branches and sphenopalatine ganglion that supply the upper third of nasal cavity close to the olfactory mucosa.

FIG. 1a is the diagrammatic presentation 100a showing vestibule 375, respiratory nasal mucosa 376 and olfactory mucosa 375 of the lateral and medial walls of the nose. The arrows point to the spread of therapeutic agents from the ORE to the CNS. Note to get the maximum delivery of therapeutic agents to ORE, the head should be extended as shown in the diagram.

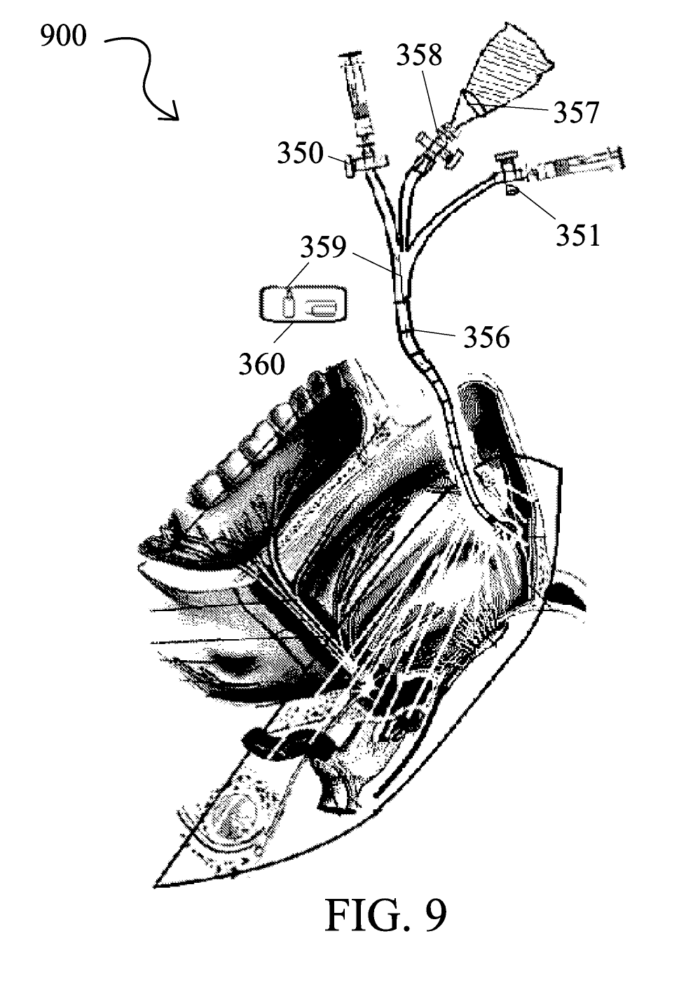

FIG. 2 is the diagram of the lateral wall of the nasal cavity 200 showing various nerve structures that the therapeutic agents with insulin of our invention come in contact and transported to the CNS retrograde through sub perineural epithelium space that surrounds the nerve fasciculi to subarachnoid space (SAS) to the cerebrospinal fluid (CSF) used in the treatment of autism. The therapeutic agents pass through the olfactory bulb 35 transported by the olfactory mucosa and olfactory nerves 105 passing through the cribriform plate of the ethmoid bone 8. The insulin and therapeutic agents are passed on to the CNS and the CSF through the trigeminal nerve 118, 109, external nasal nerve 116, and the anterior ethmoidal nerve 117, and from the sphenopalatine ganglion 110 to the greater petrosal nerve 119, nerve of the pterygoid canal 111, pterygopalatine and pharyngeal nerve 112, lesser palatine nerve 114, greater palatine nerve 115, nasopalatine nerve. The sphenopalatine ganglion neuronal center is located in the brain behind the nose (see FIG. 9). Besides the above branches, it consists of parasympathetic neurons innervating the middle cerebral and anterior cerebral arterial lumens, the facial skin blood vessels, and the lacrimal glands. Activation of this ganglion is believed to cause vasodilatation of these vessels possibly even the basilar and posterior cerebral arteries. A second effect of such stimulation is the opening of pores in the vessel walls, causing plasma protein extravasations. This effect allows better transport of molecules from within these blood vessels to surrounding tissue.

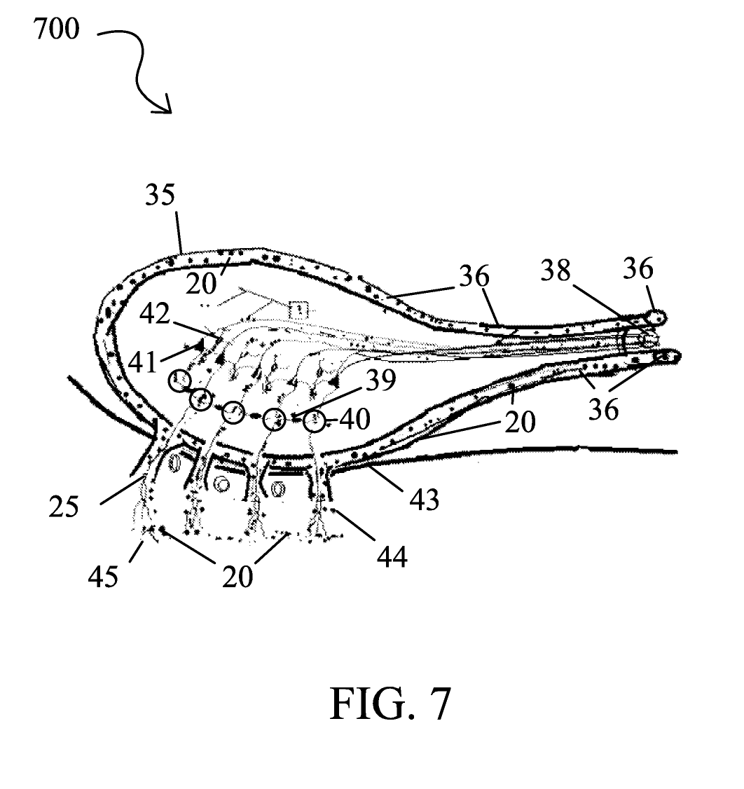

The therapeutic agents can seep on to the middle ear through the pharyngeal opening of the pharyngo-tympanic tube 113. This diagram illustrates the rich nerve plexus in the upper third of the nose called the olfactory region (ORE). Most of the therapeutic agents are conducted to the CNS through the sub Perineural epithelial space and the interstitial space between the axon bundles to SAS and CSF to reach the cerebral cortex, hypothalamus, brain stem, cerebellum and other CNS structures. The passage of therapeutic agents through the olfactory mucosal system is faster than the trigeminal and Sphenopalatine ganglion complex because of its closeness to the olfactory bulb and SAS that surrounds it (see FIG. 7). The therapeutic agents are also transported through the trigeminal nerve and reach the cranial nerve nuclei in the brain stem. The olfactory mucosa plays a major role in delivering therapeutic agents in the treatment of autism in this invention with insulin and other therapeutic agents (diagram modified after Gray's Anatomy).

FIG. 3 is the diagram of the medial wall of the nasal cavity 300 and various nerve structures that the insulin and therapeutic agents used to treat autism in this invention come in contact and are transported to the CNS retrograde from the upper part of the nose (see FIG. 1a) from the olfactory region (ORE) used for the treatment of autism in this invention. The insulin, and various known therapeutic agents, of our invention pass through the cribriform plate of the ethmoid bone 8 to the olfactory bulb 35 conducted by the olfactory mucosa 106 and olfactory nerves 105 through the sub perineural epithelial space that surrounds the nerve fasciculi to subarachnoid space (SAS); to the cerebrospinal fluid (CSF); to the pontine cistern and CSF around the optic chaisma; then to various centers of the brain, and cortex, especially temporal and prefrontal and orbital cortex; front part of the brain stem as well as to the cerebellum. The axons and dendrites of the olfactory tract does transport the therapeutic agents, but is very small doses and takes days to reach the brain centers involved in autism.

The insulin and the therapeutic agents of our invention to treat autism also passed on to the CNS and the CSF through the trigeminal nerve branches 107 and sphenopalatine ganglion 110 that supply the nasal cavity. The therapeutic agents come in contact with anterior ethmoidal nerve 107, nasoplatine nerve 109, medial, posterior and superior nasal branches 108 and the sphenopalatine ganglion 110 and its branches. The insulin and therapeutic agent's passes through these routes to reach the brain stem cranial nerve nuclei (diagram Modified from Gray's Anatomy).

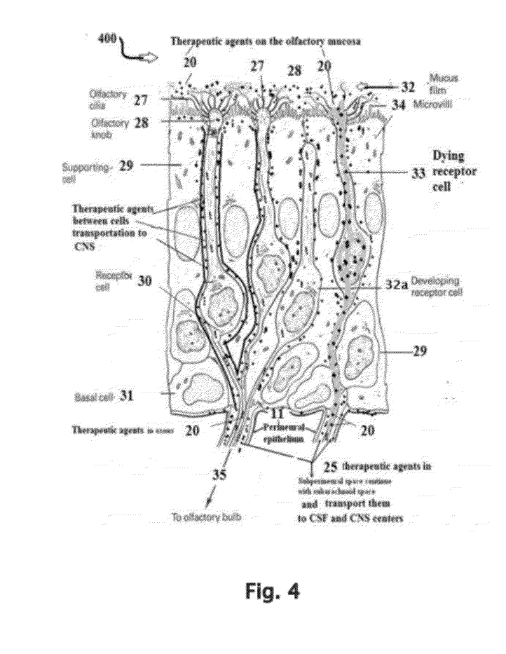

FIG. 4 is the drawing of the section of the olfactory mucosa 400, labeled with names of structures with numbering to make to understand histology of the olfactory mucosa. It is showing the route taken by the insulin and various therapeutic agents and their path of transfer to the through the olfactory nerve (±20 nerve fasciculi) to olfactory bulb and CSF in SAS of the CNS to treat autism in our invention. It shows how the insulin and therapeutic agents gets attached to the mucous film 32, entangled in olfactory cilia 27 of the olfactory cells and microvillus 34 of the supporting cells 29, and transported to through the olfactory axons 20, and Perineural epithelium 11 and sub Perineural space 25 to the olfactory bulb 35 and the SAS surrounding the olfactory bulb containing CSF (FIG. 9). Note the space created by dying olfactory cell 33, developing receptor cells 32a, and their dendritic bulb 28 can easily transmit the insulin and therapeutic agents 20 to the olfactory bulb 35 and the rest of the CNS. The basal cells 31 transfer the insulin and therapeutic agents from the surface mucosa 20 to the capillary space around the axons and to the sub perineural space below the perineural epithelium 25. There are hundreds of olfactory cells 33 dying at different locations of olfactory mucosa. This creates a space between the olfactory cells and supporting cells which makes the olfactory membrane porous like sieve creating a route for the easy transport of insulin and other therapeutic agents from the olfactory mucosal surface 20 used in our invention. The insulin and therapeutic agents are transmitted to the CNS through the axons of olfactory bulb 35 (hardly any) and sub Perineural space 25 (major route of transport) surrounding the olfactory axon bundle (fasciculi—see FIG. 6), where they enter the olfactory bulb through the cribriform plate of the ethmoid bone. (Shantha T. R. and Yasuo Nakajima. Yerkes Regional Primate Research Center, Emory University, Atlanta, Ga.: Histological and Histochemical Studies on the Rhesus Monkey (Macaca Mulatta) Olfactory Mucosa. Z. Zellforsch. 103, 291-319, 1970).

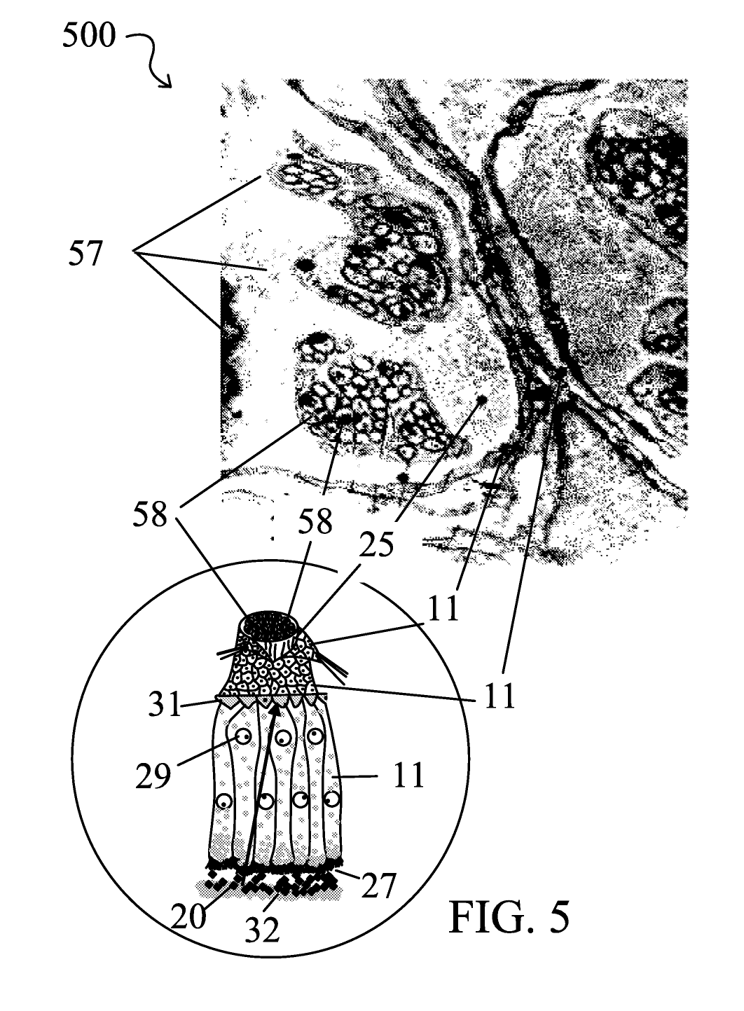

FIG. 5 are the drawing of the section of the olfactory mucosa and electron micrograph of olfactory nerve fasciculi 500, and the route taken by the insulin and various therapeutic agents and their path of transfer to the through the olfactory nerve 58 (±20 nerve fasciculi) to olfactory bulb and CSF of the CNS to treat autism in our invention. It shows how the insulin and therapeutic agents gets attached to the mucous film 32, entangled in olfactory cilia of the olfactory cells 29 and microvillus 27 of the supporting cells 29, and transported to through the olfactory axons 58, and Perineural epithelium 11 and sub Perineural space 25 to the olfactory bulb and the SAS surrounding the olfactory bulb containing CSF (FIG. 7). The basal cells 31 transfer the insulin and therapeutic agents from the surface mucosa 20 to the capillary space around the axons and to the sub perineural space below the perineural epithelium 25. There are hundreds of olfactory cells 29 dying at different locations of olfactory mucosa. This creates a space between the olfactory cells and supporting cells which makes the olfactory membrane porous like sieve creating a route for the easy transport of insulin and other therapeutic agents from the olfactory mucosal surface 20 used in our invention. The insulin and therapeutic agents are transmitted to the CNS through the axons 58 of olfactory mucosa (hardly any) and sub Perineural space 25 (major route of transport) surrounding the olfactory axon bundle where they enter the olfactory bulb through the cribriform plate of the ethmoid bone. (Shantha T. R. and Yasuo Nakajima. Yerkes Regional Primate Research Center, Emory University, Atlanta, Ga.: Histological and Histochemical Studies on the Rhesus Monkey (Macaca Mulatta) Olfactory Mucosa. Z. Zellforsch. 103, 291-319, 1970).

FIG. 6 is the histological structure of the olfactory nerve fasciculi 600 showing the very small olfactory nerve axons 58 carrying hardly any insulin and other therapeutic agents within their axons which travel retrograde to the olfactory bulb. This micrograph shows the perineural epithelium 11 surrounding the olfactory nerve fasciculi with sub perineural epithelial space 25 is the main route of transport of insulin and other therapeutic agents 57 deposited on olfactory mucosal transported between the receptors, supporting and basal cells to the sub Perineural epithelial space 25, 57 used to treat autism described in this invention directly to the CSF around the olfactory bulb and the brain SAS. (From Shantha. T. R, and Bourne. G. H, Perineural Epithelium, in GH Bourne, Ed. In Structure and Function of Nervous Tissues. Volume I. Academic Press, New York. 1968. pp 379-459; Shantha and Nakajima. Histological and Histochemical Studies on the Rhesus Monkey (Macaca Mulatta) Olfactory Mucosa. Z. Zellforsch. 103, 291-319 (1970)).

FIG. 7 is the drawing of the longitudinal section of the olfactory bulb 700 and olfactory mucosa showing the route of transport taken by the insulin and other therapeutic agents 20 by the direct deposition insulin and therapeutic agents to the CNS through the olfactory mucosa 45 transported to the olfactory bulb 35 in our invention to treat autism. The insulin and various therapeutic agents are transferred to the sub arachnoid space (SAS) 36 after passing through the olfactory mucosal nerve fasciculi. The SAS is directly connected to the sub perineural epithelial space surrounding the olfactory nerve fasciculi 25. The insulin and therapeutic agents 20 from receptor cells 44 transported through the axons retrograde through the cribriform plate of the ethmoid bone 43 to join the olfactory bulb 35. From the olfactory receptor cell axons, the drugs travel through the Glomeruli 40 to periglomerular cells 39, mitral cells 41, and granule cells 42, to olfactory tract 37, and reach the CNS 38. To pass all these obstacles of complex synapses in the olfactory bulb 35, it takes days and by then the concentration gradient of therapeutic agents may fall dramatically to achieve therapeutic index. Majority of the therapeutic effects of insulin and other therapeutic agents used in our invention to treat autism is due to the transport of the therapeutic agents to sub perineural epithelial spread 25 to SAS 36 and then into CSF of the olfactory bulb and CNS which then exert their effect on the neurons, synapses between the neurons; oligodendroglia, astroglia and microglia in the neuropil involved in the disease process of autism.

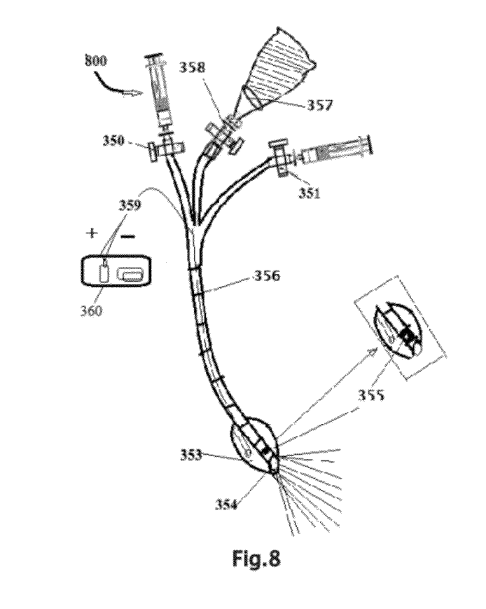

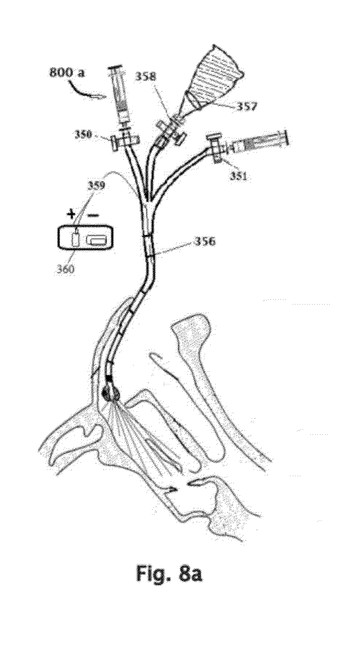

FIG. 8 is the diagrammatic presentation 800 of the special catheter delivery system to be used to deliver therapeutic agents at ORE instead of respiratory mucosa. It is made of nontoxic semi rigid-flexible catheter with 3 outlets 351, 358, 350. The outlet 351 and 358 can be used to attach any commercially available delivery sprayers and delivery devices proximally. The tip of the ORE delivery has a balloon 353 that can be inflated with air or liquids to desirable size to pass the tip through the nose all the way to the anterior part of the roof of the nose. The inflated balloon enclosing the tip prevents the trauma that can be caused by the tip of the catheter as it is introduced to the olfactory mucosal region. Further, it also holds the catheter in position without much movement when inflated. The tip is also illuminated by LED bulb or fiber optic illuminator or any other form of tip sensor 355 so as to locate the tip position of the delivery catheter in the nose. The tip or the distal end of the delivery catheter has therapeutic agent's delivery opening 354. The LED illuminator or fiber optic tip is conned to the battery power pack operated by double AA DC batteries 360 with on and off switch connected to the tip of the catheter device by positive and negative wire 359 providing the electrical power source.

As the catheter is passed on the anterior aspect of the nose, the illumination is turned on which will show the location of the tip of the catheter through the skin to be properly placed for delivery of therapeutic agents to the ORE. The catheter tip needs to be passed the nasal bone and cartilage junction so as to deliver the therapeutic agents of our invention to the ORE. The 351 and 358 catheters open to the main part of the catheter though which the therapeutic agents are delivered to ORE. Bothe these inlets can be used to delivered two or more separated therapeutic agents without mixing them. It is designed in such a fashion so that one or both inlets can be used at the same time or with lapse of time. Further, they can be attached to any delivery commercially available devices that are already commercially available. These canulas in the catheter can be used to visualize the tip by using a thing fiber optic scope and to use a guide wire to negotiate and the proper placement of the delivery catheter. The delivery catheter also has marking in millimeters on the surface of the entire length of catheter so as to indicate the how far the length of the catheter is inside the nose and prevents it being force up into mucosa so as to cause damage to the olfactory mucosa.

FIG. 9 is the diagram of the lateral wall of the nose showing the extensive neurological network in the ORE (see FIG. 1a) that come in contact with the insulin and other therapeutic agents described in our invention to transport them to CNS as described in FIGS. 7, 8a. The catheter shown in the diagram has the same explanation as FIG. 8. To prevent breakage, the catheter delivery system (and the dropper if used) needs to be made with unbreakable nontoxic, non-allergic flexible plastic or semi synthetic material. The inhalation devices described in various publications and those marketed for nasal delivery do not deliver the most of the insulin and therapeutic agents close to ORE where it is needed and effectively delivered to the CNS. The mist created by inhalation sprays by inhalation device also enters the tracheobronchial tree and deposited reparatory epithelium of the nose; the ORE receives less therapeutic agents with associated systemic reaction such as hypoglycemia due to systemic absorption by nasal and respiratory blood vessels. Note the extensive network of nerves on the upper part of the lateral wall of the nose (ORE) which carry the therapeutic agents to the central nervous system. (Modified from Gray's Anatomy)

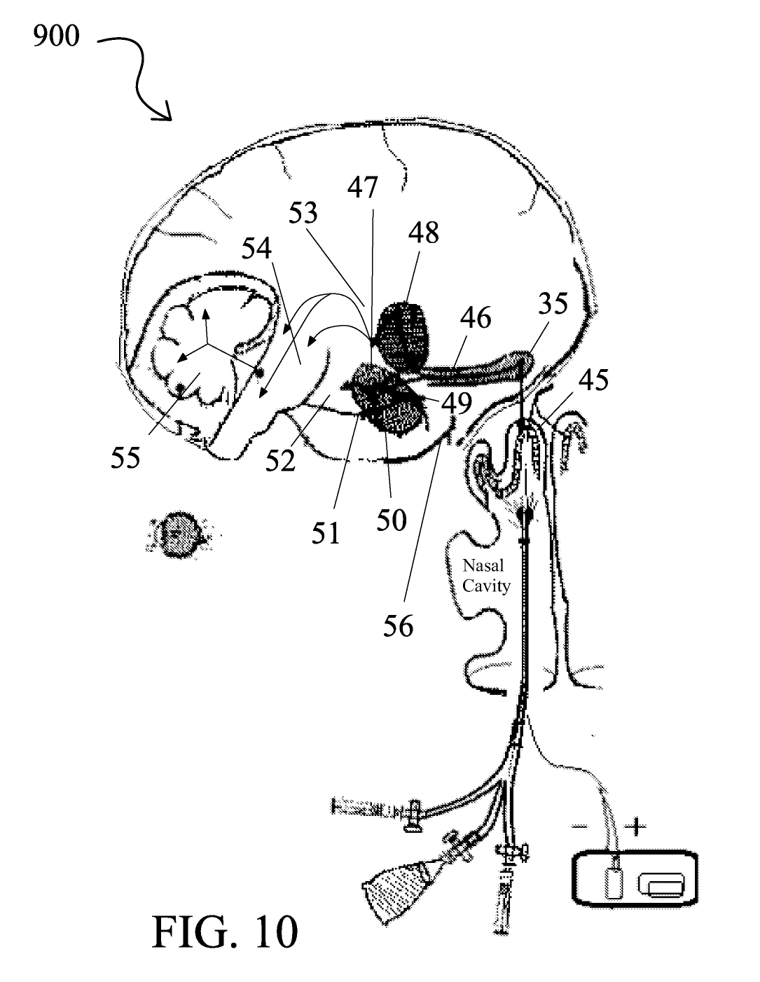

FIG. 10 is the drawing 1000 of the section of the olfactory mucosa 45 lining of the nose close to the cribriform plate of the ethmoid bone and the olfactory bulb 35 within the cranium situated immediately above cribriform plate of the ethmoid bone and the olfactory mucosa. The diagram is showing the route taken by the insulin and various adjuvant therapeutic agents' deposited at the olfactory region in our invention to treat autism. Therapeutic agents after deposition on the olfactory mucosa 45 are transported to the olfactory bulb 35 to subarachnoid space (SAS) to the cerebrospinal fluid (CSF) then to various centers of the brain and cerebral cortex, especially temporal and frontal lobes. Some of the therapeutic agents of our invention do reach the CNS through the olfactory tract 46. From the olfactory bulb, its subarachnoid space (SAS) and the cerebrospinal fluid (CSF), the insulin and therapeutic agents used to treat autism spreads to the olfactory tract 46, to prefrontal cortex 47, medial olfactory area 48, to temporal lobe 50, to lateral olfactory area 51, hippocampus 52, hypothalamus 53, brain stem nuclei 54, to cerebellum 55. From the CSF from the subarachnoid space surrounding the olfactory tract, optic chaisma, and the pontine CSF cistern; the therapeutic agents can reach the eye 56 and brain stem frontal surface where most of the cranial nerves emerge. To prevent the spread of the therapeutic agents to systemic circulation avoid using nasal sprays, and deliver the therapeutic agents needs to be deposited on the ORE and on the directly on the olfactory mucosal surface using of special delivery catheter as shown in the diagram to deliver them to the CNS.

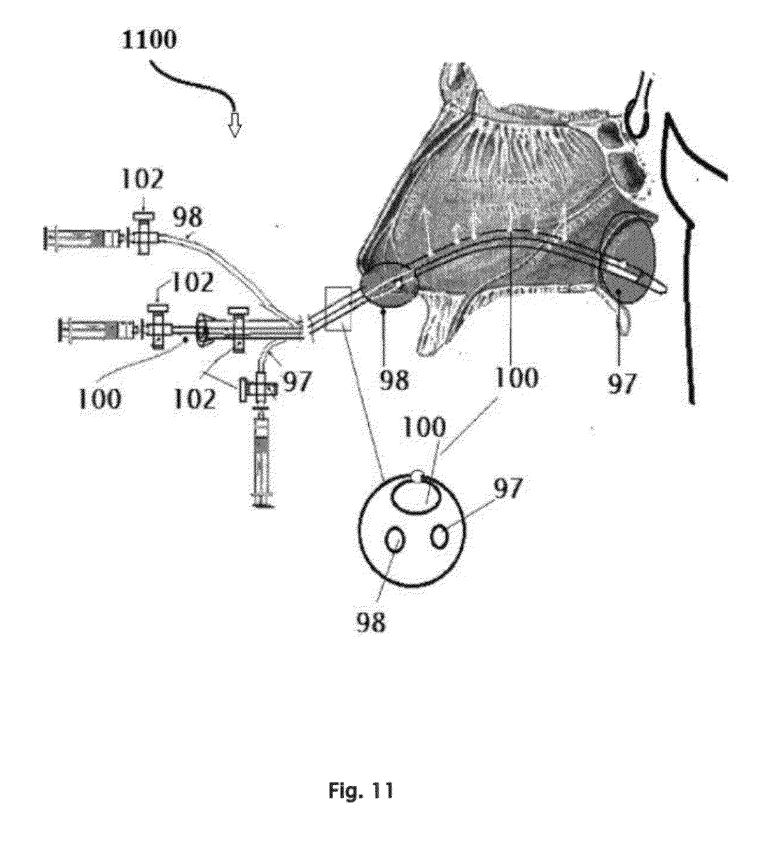

FIG. 11 is the diagrammatic presentation of the inventive device 18 to be used to deliver the therapeutic agents of our invention through the nasal cavity to the olfactory mucosal nerves, nasociliary nerve from the trigeminal ophthalmic division, nerve of the pterygoid canal, and nasoplatine nerve and the sphenopalatine ganglion and its branches to treat autism. This device helps us deliver the therapeutic agents continuously without interruption. The device is made of three canulas 97, 98, and 100; connected to the proximal end by stop cocks 102 which attaches to the syringes. The canula 97 is connected to the distal balloon as shown in the diagram, and the 98 to proximal balloon, and the 100 to the drug delivery canula with multiple openings on the intranasal length which allows the therapeutic agents to be delivered to the proximity of the above mentioned nerve structures at (olfactory nerves and other adjoining nerves) in the ORE. The therapeutic agents are absorbed by these neurological structures and transported to the CNS neuropil and to the CSF of the SAS without leaking back into nasopharynx and oropharynx. First, place the patient in a supine position with head extended over a neck support. Have suction catheter and equipment available if the patient needs to be suctioned in the nasal, oral, and pharyngeal areas. The catheter tip and the nostril are lubricated with KY jelly or other sterile Vaseline based lubricants. If the patient is awake and agitated, local anesthetic spray such as Citanest spray or xylocalne (lidocaine) jelly can be used to anesthetize the nasal surface sensory nerves. Then, gently pass the catheter at the bottom of the nose touching the floor of the nose, directly at 90 degree angle to the external naris. As it passes towards the back, it comes in contact with the wall of the nasopharynx which can be felt as obstruction for further advancement. If one continues to push the catheter, it may appear the in the oropharynx. Then, fill the distal balloon 97 with saline or air. Then, pull the catheter gently forward until it hits the posterior opening of the nose. Once it comes in contact, blow or fill the proximal balloon 98. Once you obtain a tight fit between these two balloons, the therapeutic agents can be administered gently through the syringe attached to the stopcock to the canula 100, and deliver the therapeutic agents to the nasal olfactory mucosa to cover the above mentioned nerve structures in the treatment of autism.

The capacity of each nasal cavity is estimated to be 7.5 ml. The practitioner may use up to 3.5 ml to cover the olfactory region in the supine position with head fully extended on neck pillow. If at all possible, drug doses need to be divided in half with each nostril ORE receiving half the dose; which doubles the absorptive surface area. In addition, a noteworthy difference in drug distribution is observed when various methods of medication administration are used: nose drops, plastic bottle nebulizer, atomization pump, pressurized aerosol, etc. Multiple studies show that the atomized pump is the best nasal delivery system because it gives a constant dose and a very good mucosal distribution. To reach the ORE, the tip of the spray with balloon catheter should be close to the OR. In addition, research has demonstrated that Clearance of spray is much slower than clearance of drops [Hardy, J. G., S. W. Lee, and C. G. Wilson, Intranasal drug delivery by spray and drops. J Pharm Pharmacol, 1985.37(5): p. 294-7]; since much of the spray deposits on non-ciliated areas, whereas, nose drop solutions sprays are primarily distributed on ciliated ORE surfaces and more effective than the simple sprays. Particle size of the sprays also affects distribution. With nasal breathing, nearly all particles with a size of 10-20 μm are deposited on the nasal mucosa, those less than 2 μm pass through the nasal cavity and deposit in the lungs. If drugs are introduced as soluble particles they may readily pass into the nasal lining secretions and then be absorbed into the blood or ORE routes.

The therapeutic agent's doses need to be reduced in children. The balloons can be deflated and withdrawn after the therapeutic agents are absorbed from the olfactory area of the nose. This catheter prevents the drainage of the therapeutic agents back into the pharynx and prevents swallowing or entering the larynx. It helps to contain the therapeutic agents locally in the upper part of the nasal cavity without the loss through the nasal choanal opening; i.e., that is the opening between the nasal cavity and the nasopharynx which prevents it from seeping into the pharyngeal opening of the pharyngo-tympanic tube. Choanal opening is bound, anteriorly and inferiorly by the horizontal plate of palatine bone, superiorly and posteriorly by the sphenoid bone and laterally by the medial pterygoid plates.

FIG. 12 is the drawing of the nerve fasciculi 1200 showing the structure of the peripheral nerve fasciculi, its coverings, blood vessels 303, perineural and perineural epithelial connective tissue 302, multiple layers of perineural epithelium 304, surrounding each nerve fasciculi, blood vessel traversing between the layers of Perineural epithelium 305 to form Virchow-Robin space 306. The Virchow Robin space surrounds the BV 303 as they enter the nerve fasciculi for a very short distance 306. Note the distinct sub perineural epithelial space below the perineural epithelium covering of the nerve fasciculi 307 which communicates with the interstitial space around each axon 309 surrounded by scanty delicate endoneurium and thick myelin sheath. Each axon is surrounded by minimal endoneurium 308.

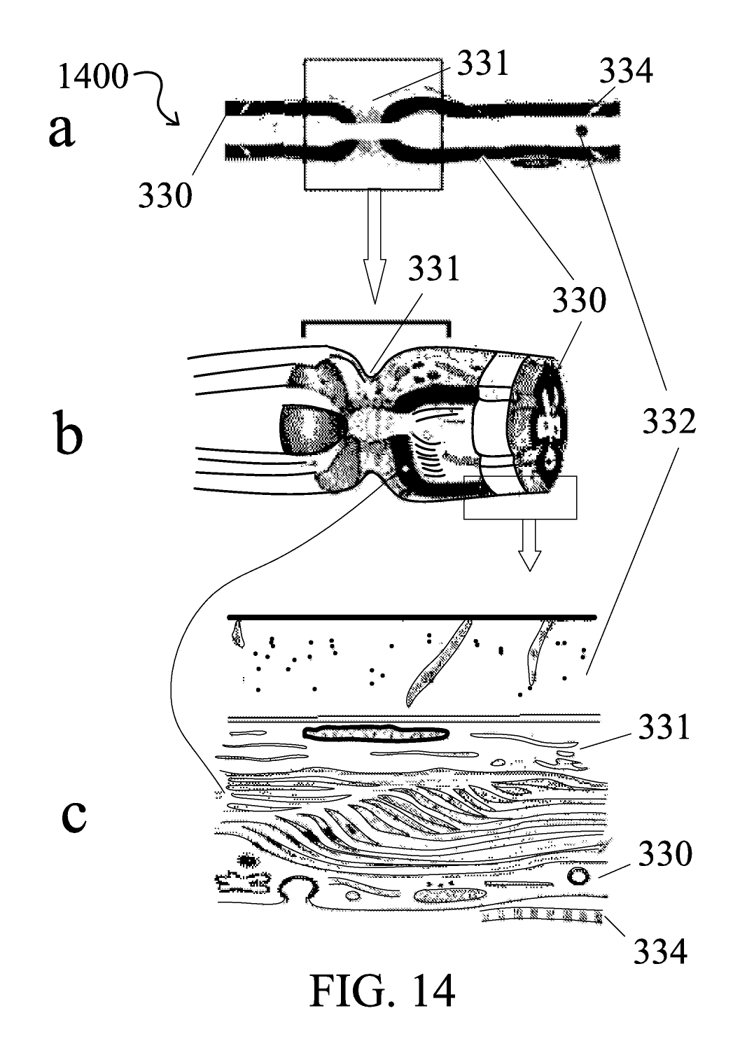

The mechanism of transfer of the insulin and therapeutic agents used in our invention to treat autism has to enter the inside the nerve fasciculi to be transported retrograde to the CNS by the axons, the therapeutic agents has to pass through the nerve fasciculi connective tissue (epineurium), perineural epithelium, Virchow-Robin space, and sub perineural epithelial space, then, pass on to sub perineural epithelial space and interstitial space between axons. From these spaces, the insulin and other therapeutic agents used to treat autism in our invention enter the node of Ranvier and then enter the axoplasm to be transported retrograde by axoplasm (see FIG. 14). The insulin and other adjuvant therapeutic agents in the sub perineural epithelial and interstitial space in the nerve fasciculi are transported retrograde to the SAS and CSF of the CNS. These are the major route of transport of therapeutic agents to CNS and the axonal transport plays a very minor role. From here, the insulin and other therapeutic agents are distributed to the surface of the brain from where they enter the neuropil to treat autism. That is how the insulin and therapeutic agents spread to the CNS from the trigeminal nerve branches and sphenopalatine ganglion of the ORE (after Shantha, Virchow-Robin space in the peripheral nerves, 1992, ASRA March-April Supplement).

Once the therapeutic agents are inside the nerve fasciculus in the edoneural surroundings, the insulin and other therapeutic agents can enter the axons at two sites: 1. They can enter the unmyelinated small axons surrounded by Schwann cells without myelin, but not through the myelin of the most peripheral axons in the nerve fasciculi, and 2. The insulin and therapeutic agents can enter the axoplasm only through the Node of Ranvier in a thickly myelinated axons which is metabolically active site on the axons, lacking insulating permeability resistant myelin sheath. The myelin sheath surrounding the axon is almost impermeable to insulin and other therapeutic agents.

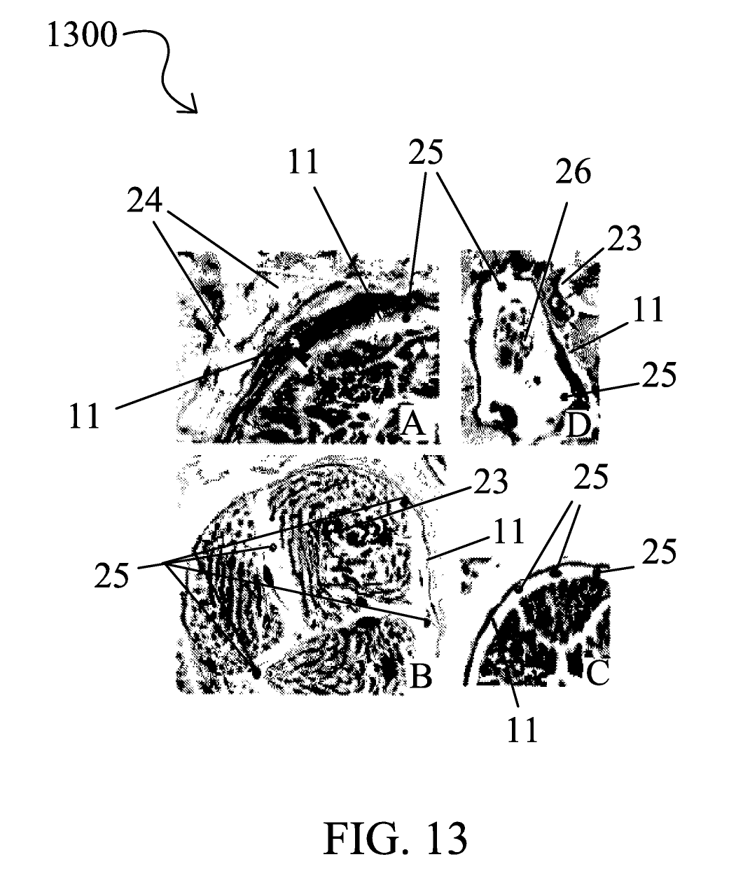

FIG. 13 Histological Section of a nerve fasciculi A, B, and C showing strong lactic dehydrogenase activity in the perineural epithelium cells (arrows) whereas the perineural connective tissue shows negligible activity 24. The axons show strong positive activity whereas the myelin sheath shows negligible activity. The Schwann cell cytoplasm also shows positive activity for this test. Note that the nerve fasciculi are surrounded by perineural epithelium and form the sub perineural epithelial space below it 25. Some of the perineural epithelium cells split the nerve fasciculi also. The sub perineural epithelial space surrounds the nerve bundles and communicates with the interstitial space surrounding the axons with their endoneurium surrounds (X 275). FIG. 12 B. Rat trigeminal nerve section showing alkaline phosphatase activity in perineural epithelium cells (long arrows). Note the peeling off of the innermost layer of these cells (short arrows) which enter to form the perineural septa, thus subdividing the large nerve fasciculus. The sub perineural epithelial space 25 is formed around the nerve fasciculi by the perineural epithelial sheaths (X 275). FIG. 12 C. is the cross section of the trigeminal nerve showing strongly ATPase-positive PE sheath (arrows) which surrounds the nerve fasciculi (. X 275). FIG. 12 D. Transverse section of the denervated muscle spindle showing adenosine triphosphatase (ATPase) activity in the capsular perineural epithelium cells of muscle spindle (big arrows) as well as in the PE cell covering (small arrows) of the extrafusal nerve fasciculus (E). Note the large sub perineural epithelial space created by the perineural epithelium cells 25 which encloses the muscle spindle completely (X 300). These 4 histological sections demonstrate the presence of sub perineural epithelial space in every nerve fasciculi including the muscle spindle which is connected to the SAS of the CNS and play an important role in transmission of the therapeutic agents administered at the ORE.

FIG. 14 is the Histological diagram 1400 drawn after extensive light and electron microscopic study of the myelinated nerve axons within the nerve fasciculi. It shows the longitudinal section of a myelinated axon (a) which forms the nerve fasciculi of the peripheral nerves. Diagram 14 a, b, and c shows the node of Ranvier 331 and the rest of the nerve fiber surrounded by the endoneurium 334, almost impermeable myelin sheath 330 with cytoplasm of Schwann cell 333, and axoplasm 332 which may transport (retrograde) minimum doses of insulin and other therapeutic agents to CNS. The insulin and therapeutic agents which enter the axoplasm has to enter the axon through the node of Ranvier 332 which does not have the myelin sheath to block the therapeutic agent's entry into axons. The rest of the axon of the myelinated nerve fiber is not easily permeable to insulin and other therapeutic agents used in our invention to get into axoplasm to be transported to the CNS. That is why axoplasm plays a minor role in spread of insulin and other therapeutic agents to CNS; and, most of the therapeutic agents are transported through the sub perineural epithelium space and interstitial spaces within the nerve fasciculi. Our studies have shown that the subperineural epithelium space is the direct continuation of the SAS and CSF of the CNS and the perineural epithelium is the extension of pia arachnoid mater from the CNS to the peripheral nervous system. Insert b shows the details of the metabolically active node of Ranvier lacking myelin sheath; and allow the absorption of insulin and therapeutic agents into axoplasm from the interstitial space. Insert C is the section of the rest of axon with thick myelin sheath covering is an obstacle for easy uptake of insulin and therapeutic agents into axoplasm. Hence, once inside the nerve fasciculi, the insulin and the adjuvant therapeutic agents enter the axoplasm at node of Ranvier 331 mostly, to be transported retrograde to the CNS. Majority of the insulin and other therapeutic agents administered locally to treat autism at ORE; are conducted to CNS through the sub Perineural epithelial and interstitial spaces within the nerve fasciculi to SAS and CSF of the CNS.

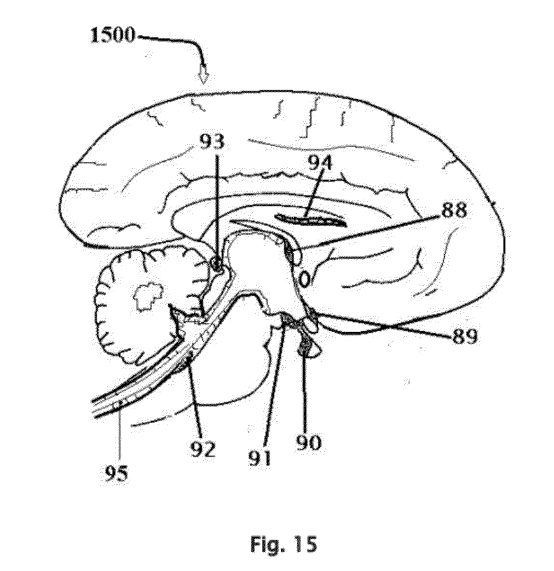

FIG. 15 is the drawing of the location of the circumventricular organs 1500 which also plays a role in hematogenous and CSF spread of the therapeutic agents of our invention to the CNS neuropil from the systemic circulation, ORE, to SAS CSF. There are several areas of the brain known as “circumventricular organs” (CVO) where the blood brain barrier (BBB) is weak and allows substances in the CNS blood vessels to cross into the brain and CSF freely with least impediment compared to the blood vessels with BBB within the neuropil of the CNS. The term neuropile (neuropil) in the following description refers is an intricate, complex network of axonal, dendritic, glial cells arborizations, and Microglial cells. There are 400 miles of capillaries with BBB that forms the bulk of the central nervous system's gray matter where the nerve cell bodies lay surrounded and embedded. The white matter is mostly composed of axons and glial cells that are generally, not considered to be a part of the neuropil. The insulin and therapeutic agents delivered to the ORE can be transported through the Lympho-haematogenous spread to the CNS through vascular plexus of CVO, and those that communicates with the ORE with the CNS, cranial nerves, spinal nerve roots, and Bates plexus of veins, ophthalmic venous plexus, and circumventricular organs.

The circumventricular organs are where the therapeutic agents can enter the CNS, through the systemic circulation, SAS and CSF. They include: Pineal gland 93 which secretes melatonin and associated with circadian rhythms; Neurohypophysis (posterior pituitary) 90 that secretes oxytocin and vasopressin into the blood to maintain BP and the urine output; area postrema 92, a chemo sensitive vomiting center in the fourth ventricle of the brain stem; subformical organ 88, involved in the regulation of body fluids; vascular organ of the lamina terminalis 89, a chemosensory area, detects peptides and other molecules; median eminence 91 regulates the anterior pituitary through release of neuro hormones, including choroid plexus 94, Ependymal lining of the ventricles and Central canal 95 with tanycytes extensions to neuropil (see FIG. 20); arachnoid villi of the sagittal sinus and spinal nerve roots, pia mater of the brain and spinal cord, optic nerve with SAS with arachnoid villi; the emerging nerve roots of the CNS and spinal cord (Shantha T R and Evans J A: Arachnoid Villi in the Spinal Cord, and Their Relationship to Epidural Anesthesia. Anesthesiology 37:543-557, 1972. Shantha T R and Bourne G H: Arachnoid villi in the optic nerve of man and monkey. Expt Eye Res 3:31-35 (1964). Nakajima Y, Shantha T R and Bourne G H: Histological and Histochemical studies on the subformical organ of the squirrel monkey. Histochemie 14:149-160 (1968). Manocha and Shantha. Enzyme Histochemistry of the Nervous System (Macaca Mulatta, 1970, Academic Press, 18-305).

Studies on experimental animal's doe's show that the rabies viremia can spread directly to the brain through the circumventricular organs without spreading retrograde through the axons through the peripheral nerves as once perceived (Preuss M R, et al. (2009). Hematogenous spread of rabies virus through Circumventricular organ. PLOS Pathog. 5(6): 1000485.doi:10. 13711journal.ppat.1000485). Likewise, insulin and other therapeutic agents from the BV of the ORE and CSF enter the CNS neuropil through these circumventricular organs. Besides ependymal lining, pia mater linings, Virchow Robin space, arachnoid villi, nerve roots emerging from the brain and spinal cord, venous blood vessels of Bates, nerve root lymphatics, and the choroid plexus also play a role in transport of insulin and therapeutic agents to CSF and neuropil.

It is important to note that the BV of the olfactory region (ORE) and the eye ball are in direct communication with the cavernous venous plexus, around the pituitary gland, petrosal veins and other tributaries of intracranial veins, which communicate with the CNS at the neurovascular interface of the hypothalamus-hypophysis system and with complex venous sinuses within the cranium, neuropil and subarachnoid space. From these sites, insulin, insulin-like growth factor-I (IGF-1) and other adjuvant therapeutic agents can spread from the ORE, OM or the conjunctival sac of the eyes, uveal vascular system, and retro bulbar venous plexus to the CNS through various weak BBB systems of circumventricular organs linings, and complex communicating venous network to neuropil of the cerebral cortex and brain stem and convey the therapeutic agents in the treatment of ASD.

The possibility of spread of insulin and other adjuvant therapeutic agents through CVO (see FIG. 15), which are highly vascularized sites, that facilitate direct communication of neurons with blood and liquor through fenestrated endothelium of the BV in these areas does exists. CVO either consist of neuronal cell bodies that sense various circulating substances (sensory CVO), or they are formed by neurosecretory axons and glial cells (secretory CVO). Their special composition exposes them as targets for invasion of pathogens and trypanosome (Schultzberg M, Ambatsis M, Samuelsson E B, Kristensson K, van Meirvenne N (1988) Spread of Trypanosoma brucei to the nervous system: early attack on circumventricular organs and sensory ganglia. Neurosci Res 21: 56-61); as well as to other insulin and therapeutic agents in the treatment of autism described in our invention. There is a possibility of retrograde spread of various known therapeutic agents, as well as other pharmaceutical, biochemical, nurticeuticals, and biological agents or compounds used for the treatment of ASD of our invention, from vessels from the ORE, OM, and the eyes into the CNS through Neurosecretory fibers of the CVO of median eminence.

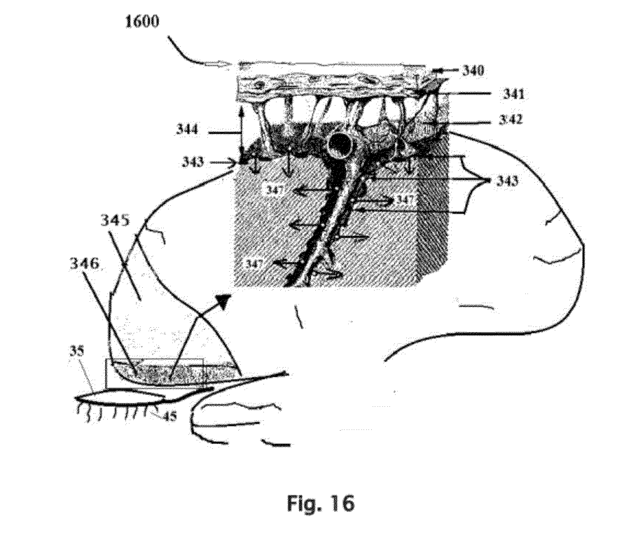

FIG. 16 is the diagram 1600 of the Virchow-Robin space in the central nervous system. It shows the olfactory bulb 35 and olfactory mucosa 45 delivers the insulin and other therapeutic agents to the SAS 344 into the CSF. The CSF is the fluid media which spreads the ORE delivered therapeutic agents. Once in the CSF, it enters the neuropile through the pia mater, Virchow-Robine spce, pial covering, CVO, and through the penetrating blood vessels in the neuropil. This diagram shows dura mater 340 located immediately below the skull bones, arachnoid mater 341, sub arachnoid space 344 with CSF, pia mater 343 extending on the blood vessel deep into the cortical part of the brain, brain stem, and spinal cord to from the Virchow-Robin space 347. The CSF permeates this space down into surface of the CNS (arrows), and let the therapeutic agents percolate and permeate all through the neuropil and back to central canal of the spinal cord and ventricles of the brain. The therapeutic agents also enter the brain neuropil through the blood vessel and pial absorption of insulin and other therapeutic agents from the CSF of the SAS as they pass through these spaces. The therapeutic agents absorbed through the BV are unable to deliver much of the therapeutic agents due the presnec of BBB unless it is breached artificially. Some of the insulin, IGF-1 and other therapeutic agents of our invention enter the delicate BV as they pass through the SAS to enter the brain. This diagram also shows the prefrontal 345 and pre supraorbital 346 cortex which is affected in the autism is located close to the olfactory bulb where the therapeutic agents of our inventions are delivered.

Most of the CSF in the brain is located in the pontine cistern and cisterna magna and the rest of CSF surrounds in a capillary thin SAS covering cerebral hemispheres, cerebellum and spila cord as well as the optic nerve. The various known therapeutic agents, as well as other pharmaceutical, biochemical, nurticeuticals, and biological agents or compounds with insulin and insulin-like growth factor-I (IGF-1) in the treatment of autism in our invention passed through the SAS in front of the brain and brain stem CSF (small arrows) from the olfactory bulb 35 and olfactory mucosa 45, trigeminal pathways, and sphenopalatine ganglion connections. Hence, the Virchow-Robin space 347 delivers the insulin, Insulin-like growth factor-I and adjuvant therapeutic agents to these regions rapidly. It is the delivery of the insulin and therapeutic agents through the Virchow-Robin space 347 and pial membrane 343 deep into the surface of the CNS responsible for the therapeutic effect to cure and/or curtail autism. That is how the insulin and the other therapeutic agents from the ORE reach the CNS and exert their therapeutic effect (diagram modified from Grays Anatomy).

Virchow-Robin spaces 347, or enlarged perivascular spaces are spaces (often only potential) that surround blood vessels for a short distance as they enter the brain 347, spinal cord and peripheral nerves (see FIG. 12 #306). Their wall is formed by prolongations of the pia mater in the CNS and perineural epithelium in the peripheral nervous system (one cell thick). The spaces function as pathways for the transfer of insulin, Insulin-like growth factor-I and other therapeutic agents to enter deep into the surface of the brain and drainage of interstitial fluid from the neuropil. The Virchow-Robin space in the CNS 347 is direct connection with the subpial space, are separated by a single layer of pia mater 343 from the subarachnoid space.

The brain and the spinal cord are bathed in cerebrospinal fluid (CSF) 344 which carries the therapeutic agents of our invention inside the brain. CSF is secreted by the choroid plexus in lateral, III^{rd}, and IV^{th} ventricles in the brain and via the weeping or transmission of tissue fluid by the brain into the ventricles. From here, the CSF percolates down the cerebral cortex ventricles, brain stem, and the spinal cord in the space between the pia and arachnoid mater (SAS). The overflowing CSF empties into the blood of the venous sinuses via the arachnoid villi in the sagittal sinuses, intracranial vascular sinuses, optic nerve and spinal nerve root arachnoid villi and, thereby potentially delivering a protein transported to the SAS and neuropil via he ORE and ear to the central nervous system.

The average human has 100-150 ml of CSF, 20% of which is located in the brain ventricles, 20% in the subarachnoid space (underneath the pia), and 60% in the lumbar cisterns of the spinal cords. The choroid plexus produces approximately 450 ml of CSF per day, about 21 ml in adults and 10 ml in children per hour, enough to replace the CSF contents 3 to 4 times a day. CSF flows from the choroid plexus into the lateral ventricles, through the interventricular foramen of Monroe, into the third ventricles, out the cerebral aqueduct of Sylvius, and into the fourth ventricle. It then moves out the foramen of Lushka and Magendie into the pontine cisterns and cisterna magna (the spaces below and above the brainstem and upper cervical spinal cord).

In the human, the dura 340 is thick and opaque whereas, the arachnoid 341 and pia 343 is thin and translucent. The CSF occupies the subarachnoid space 344. When a person is lying down, the CSF pressure is 4-16 mm Hg; the pressure of course increases as the person sits up, since the pressure reflects the column of fluid. CSF pressure is also influenced by venous pressure and typically pulsates with breathing and heartbeats. Average CSF movement in the posterior spinal subarachnoid space is towards the tail while the average CSF movements in the anterior spinal SAS space and central canal tend to be toward the head which might be due to effect of hearts and lungs pulsatile force and denticulate ligament. That is why the therapeutic agents from the ORE come in contact with the fore part of the cerebral cortex, front of the brain stem and stay in contact with these areas of the brain longer period of time in our method of the treatment of autism using of insulin and other adjuvant therapeutic agents. Therefore, intrathecally administered drugs in the posterior subarachnoid space move downward towards the caudal (tail ward) spinal cord and then back towards the rostral (head ward) end of the cord.

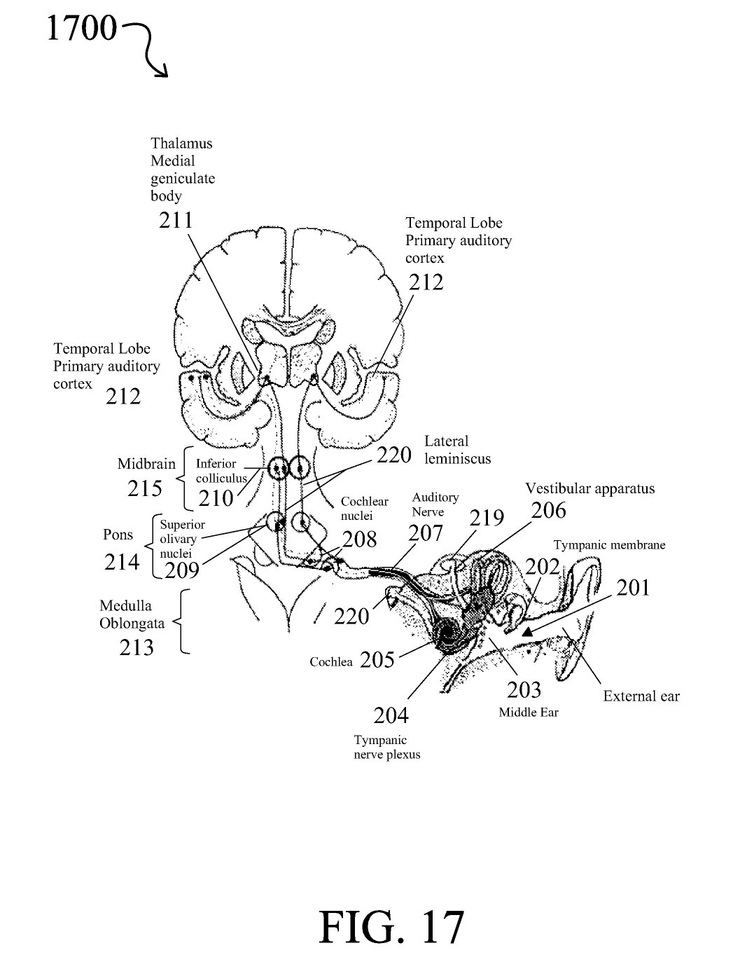

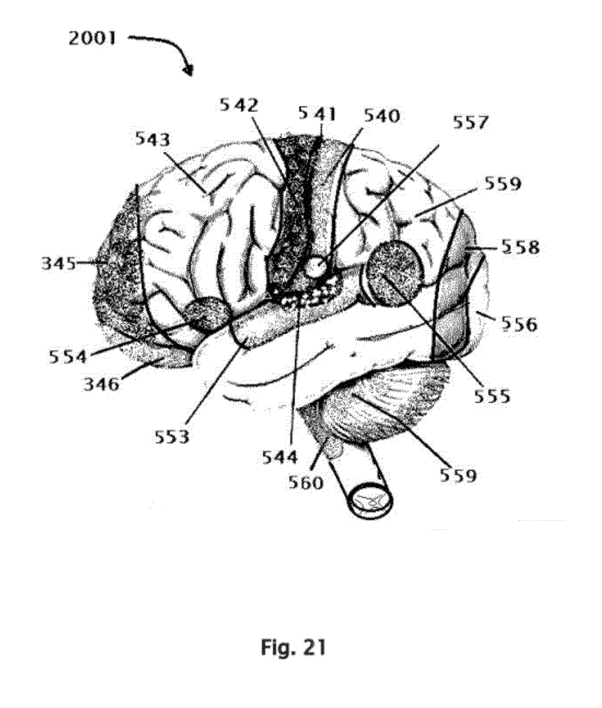

FIG. 17 is the drawing 1700 showing the nerve route involved in the conduction of the auditory impulses from outside to be perceived as sound in the CNS. This diagram also shows how the insulin, IGF-1 and other therapeutic agents from the external ear used in the treatment of autism in our invention are also transported to the middle ear, inner ear and the auditory pontine nuclei to exert therapeutic effect on these areas. There are studies which indicate that there is milliseconds of conduction delay of the sound waves to the CNS auditory centers in autism. The Insulin-like growth factor-1, insulin and other therapeutic agents our invention will prevent this delay and establish normal conduction improving the communication skills of the autism patients. This auditory pathway acts as a conduit for transport insulin, Insulin-like growth factor-I, and other therapeutic agents applied to the external auditory meatus 201, which are transported through the tympanic membrane 202 to the middle ear 203 then to rich network of tympanic nerve plexus on the medial wall of the middle ear 204 (arrows). From there, the insulin, Insulin-like growth factor-I (IGF-1) and other adjuvant therapeutic agents are transported to the cochlear (auditory) hair cells and cochlear nerve 207 (through endolymph, and perilymph); facial nerve, glossopharyngeal nerve, chorda tympani, and vestibular nerve to the Ponto-medullary junction where the cochlear nerve enters to relay neurons are located. Many of these nerve fibers in the pons synapse and cross over to opposite side, synapse in the superior olivary nuclei 209, ascend up in the pons 214 and midbrain 215 in the lateral lemniscus 220 to reach the inferior colliculus 210, thalamic medial geniculate body 211 and finally reach the temporal lobe 212 which is the primary auditory cortex (see FIG. 21) which perceives the sound. The cells of the dorsal part of the olivary nucleus are primarily GABAergic, (Adams, J. C. and E. Mugnaini (1984). “Dorsal nucleus of the lateral lemniscus: a nucleus of GABAergic projection neurons.” Brain Res Bull 13(4): 585-90) and projects bilaterally to the inferior colliculus. Hence, the therapeutic agents of our invention from the ears can reach this nucleus easily to have therapeutic effect on the symptoms of ASD.

Important aspect is that the insulin, IGF-1 and other therapeutic agents are also transported to the SAS, through the endolymphatic duct and sac 219 from the macula sacculi-utriculi and perilymphatic canaliculi 220 from cochlea also which enter the brain stem nuclei to have a therapeutic effect in the treatment of autism.

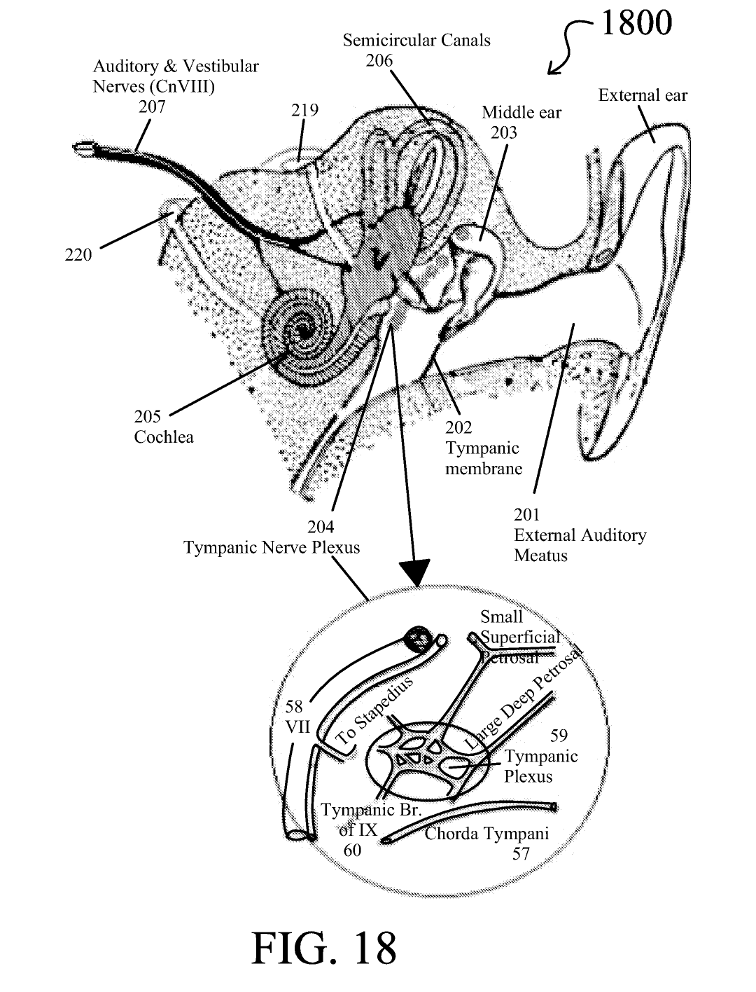

FIG. 18 is the drawing 1800 of the external, middle and internal ear involved in conduction of sound waves and transport of insulin, IGF-1 and other therapeutic agents administered for the treatment of autism through the tympanic plexus. These therapeutic agents pass through the external auditory meatus 201, thorough the tympanic membrane 202 and vascular plexus to the middle ear 203. The middle ear has rich network of nerve plexus located on the medial wall of the middle ear 204 and on the tympanic membrane called the tympanic plexus. The cranial nerve facial, glossopharyngeal, cochlear and vestibular nerves are connected through the middle ear and middle ear tympanic plexus 204. Though the IGF-1, insulin and therapeutic agents are carried to the cochlea through the found and oval window to the hair cells nadn to the CNS through the tympanic plexus, the main nerves which carry the insulin, IGF-1 and other therapeutic agents are cochlear 202, and vestibular nerves 206 through cranial nerve VIII 207. The therapeutic agents used to treat autism in our invention are transported to upper part of the medulla oblongata and pons through the relay centers or neuron of cranial nerve VII, and IX. They are transported to the brain stem through the SAS, and sub perineural epithelial spaces via CSF as described in the FIGS. 3-12. These IGF-1, insulin and therapeutic agents of our invention have an effect on conduction of nerve impulses to the cortical centers, and at pontine relay. The effect is such that the delay in synaptic transmission noted in various studies in autism patients is cut down, and normal conduction is established. Note the complex nerve plexus on the medial wall of the middle ear with facial nerve 58, tympanic branch of the glossopharyngeal nerve 60, chorda tympani 57 with large deep petrosal 59 and small petrosal nerves. The insulin, IGF-1, and therapeutic agents used to treat autism are transported to the SAS close to the pons, through the endolymphatic duct and sac 219 from the macula sacculi-utriculi and perilymphatic canaliculi 220 from cochlea, also.

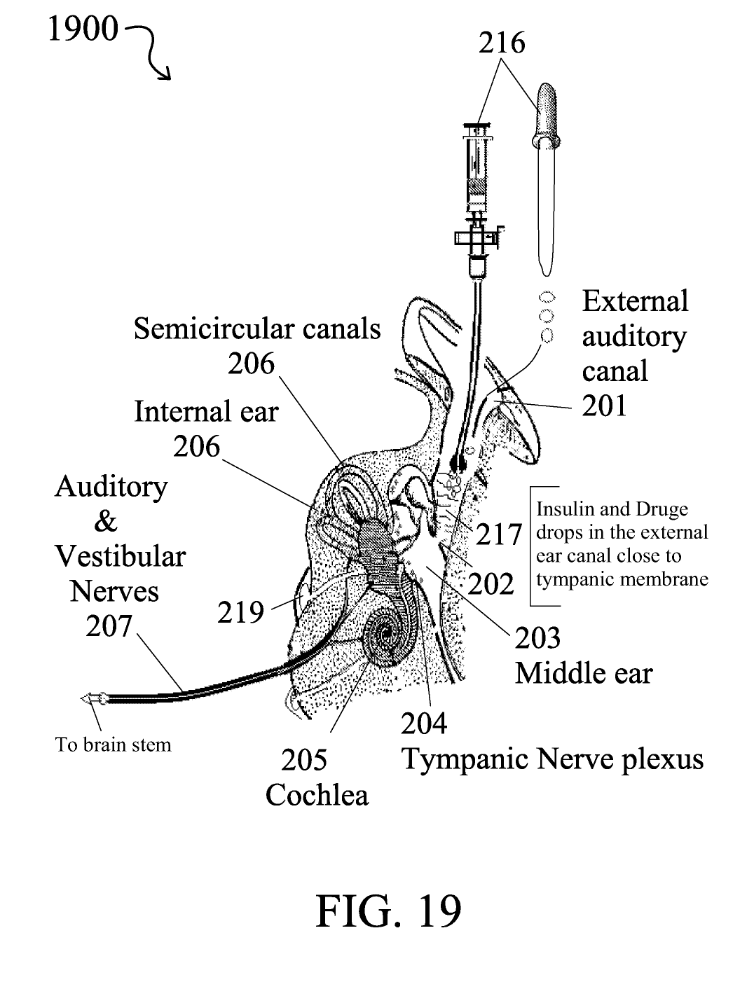

FIG. 19 is the drawing 1900 of the ear with patient left lateral position with delivery of therapeutic agents through the ear to treat autism. Explanation is the same as FIG. 18. Note the external auditory meatus 201 is facing upwards with the medication syringe or dropper with IGF-1, insulin and therapeutic agents instilled close to ear tympanic membrane 202 which is absorbed to the middle ear and transported to the tympanic nerve plexus and hearing apparatus (cochlea 205) then conducted to the CNS brain stem nuclei. The therapeutic agents soaked cotton pledge or gel can be deposited close to the tympanic membrane with string attached to pull it out after 2-4 hours of application for continuous slow absorption of therapeutic agents used in our invention.

FIG. 20 is the diagram 2000 of the neruopil 367 between the ependymal linging of the central canal and ventricle 361 and the SAS 344 surrounding the brain (CNS) and the spinal cord. Note the epednyma lining 361 of the central canal and vetricles giving rise to tanacyes 362 which is branching and coming in contact with the neurons 364 and the rest of the neuropil. The diagram also shows the microglia 362 in the neuropile, astroglial 363 end feet surrounding the BV 365 to form BBB along with the pericytes and amorphous non cellular complex surrounding the BV to form solid BBB. It also sends end feet to attach to the undersurface of the pia mater, and to come in contact with the ependymal lining and tanacytes 361,362. The end feets of astroglia also surround the neuronal cell body and their processes 363. The ologodendroglia 366 send multiple extensions to surround the axons and form myelin sheath in the central nervous system akin to the Scwann cells in the peripheral nerves. Note the thin, one layer thick pia mater 343 which is lined by astroglial end feet 363 towards the neuropil. Pia mater is carried into the cortex of the brain along with the penetrating BV 342 from the SAS of the spinal cord and CNS to form the Virchow-Robine space 347 (see FIG. 16-#347). The CNS is surrounded by CSF in the SAS formed by the pia 343 and arachnoid mater 341 which is in turn surrounded by thick almost impermeable dura mater 340 firmly attached to the inside of cranial bones. The dura mater contains the large venous sinsus draining the CNS blood out of the brain to the jugular system. The neuropil 367 is shown with various neurons with nerve process, the blood vessels 365 endowed with BBB, microglia 363, astroglia 363; oligodendroglia 366 and extensions of ependymal cells as tanacytes 362. The CSF in the central canal and ventircles 360 and in the SAS 344 with insulin and other therapeutic agents of our invention permeates the neuropile through the Virchow-Robine space (see FIG. 16-347), tanacytes 362, ependymal cells 361, CVO, blood vessels, circumventricular organs and BV 365 carrying the insulin, IGF-1 and other pharmaceutical, biochemical, nurticeuticals, and biological therapeutic agents or compounds to the neuropile 367 to treat ASD as described in our invention. This diagram illustrates how the therapeutic agents of our invention reach their destination from the ORE to exert their therapeutic effect to cure and/or curtail signs and symptoms of ASDs (diagram based on Grays Anatomy).

FIG. 21 is the drawing of various regions of the cerebral cortex 2001 involved in autism. It shows prefrontal area 345 of the frontal lobe of the brain with prefrontal supra orbital cortex 346, which play a major role in autism. Note how close they are to olfactory bulb though which the therapeutic agents of our invention are delivered. The auditory cortex 553, primary auditory cortex 544, motor speech area 554, primary auditory cortex 544, secondary speech area (Wernicke's area) 555, visual cortex 556, visual association area 558, somesthetic association area 559, taste area 557, primary somesthetic (sensory) area 540, central sulcus 541, primary motor cortex 542, premotor frontal lobe 543 behind the prefrontal area 345, with prefrontal supra orbital cortex 346. It is the prefrontal area that is mostly affected in the autism patients according to latest study. This diagram also shows cerebellum 561 with vermis and its Purkije cells, and brain stem 560 which are also said to be involved in ASD pathophysiology. Our invention delivers the insulin and various therapeutic agents from the ORE close to this prefrontal area which is affected in autism in the treatment of this ASD. From the cerebro-pontine CSF cistern, and the cochlear nuclei (external ear delivery of therapeutic agents) the therapeutic agents of our invention are transported to the cerebellum and brain stem nuclei. Our invention will be effective due to nearness of this affected area of the brain to therapeutic agent's delivery route.

Respiratory Nasal and Olfactory Mucosal Anatomy, Histology and Physiology Involved in Delivery of Therapeutic Agents To the CNS to Treat Autism in Our Invention

Olfactory Region administration of insulin and various therapeutic agents developed to treat autism of our invention has many of the above described advantages when compared to oral or systemic administration. This is possible because of the unique connections that the olfactory, sphenopalatine ganglion and trigeminal nerves (ORE) and the communicating blood vessels in the vicinity provide transport connection between the brain and olfactory mucosal area (ORE).

To understand the absorption mechanism, transportation pathways, distribution of therapeutic agents administered to ORE to transport to the CNS by the intranasal route (IN), knowledge of the nasal anatomy, histology, and physiology are essential. The nose has two cavities with the middle nasal septum dividing the nose into two almost identical nasal cavities. The volume of each cavity is about 7.5 mL with a surface area of 75 cm (Mygind N, Anggard A. Anatomy and physiology of the nose-pathophysiology alterations in allergic rhinitis. Clin Rev Allergy 1984; 2:173-788; Illum L. Transport of drugs from the nasal cavity to the central nervous system. Eur J Pharm Sci 2000; 11:1-18). There are three distinctive functional regions in the nose: 1. Vestibular 375, 2. Respiratory 376, and 3. Olfactory 375 (FIG. 1a). Of these, the respiratory region 376 is the most important for systemic drug delivery and olfactory mucosal region 377 for CNS drug delivery for various diseases including for treatment of autism. The vestibule 375 is the passive passage for the entry and exit of the air to and from the lungs and is hardly involved in delivery of therapeutic agents. It is the olfactory mucosal region (ORE) which plays an important role in transport of various known therapeutic agents, as well as other pharmaceutical, biochemical, nurticeuticals, and biological agents or compounds to the CNS to treat autism and other neurodegenerative diseases. When the term olfactory region (ORE) 377 which participates in the transport of therapeutic agents is used it includes:

1. Upper part of the medial wall of the nose (FIG. 2) containing:
a) olfactory mucosal surface (OM) with olfactory receptor neurons,
b) sphenopalatine ganglion and its multiple connections,
c) sphenoethmoidal recesses on the later wall,
d) superior turbinate with superior ethmoidal recesses,
e) upper most Surface of the middle turbinate,
f) anterior part of the olfactory mucosal surface with anterior ethmoidal nerve,
g) cribriform plate of the ethmoid bone, and
h) cavernous plexus of blood vessels connecting these areas with the cavernous sinus around the pituitary, olfactory bulb, fore part of the brain, upper part of brain stem, eyes, and subarachnoid space.
   2. Upper ⅓ of the medial wall of the nasal septum medial to the above described structures of the lateral wall containing the above described nerve supply, olfactory mucosa, and blood supply (FIGS. 1-3).

The respiratory epithelium consists of basal, mucus-containing goblet, ciliated columnar and non-columnar cell types. Additionally, each cell in this region is covered by 300 microvilli, providing a large surface area for absorption of therapeutic agents. Below the epithelium is lamina propria containing blood vessels, nerves, serous, and mucus secretory glands along with the capillary network and they are permeable for drug absorption. The nasal epithelium is covered by a mucus layer with a pH of 5-6.5, and is renewed every 10 to 15 minutes (Chien Y W, Chang S F. Intranasal drug delivery for systemic medication. Crit Rev Ther Drug Carrier Syst 1987; 4:67-194). The nasal cavity has cytochrome P450 enzyme isoforms, carboxylesterases and glutathione S-transferases.

On the other hand, the ORE of the nasal cavity plays an imorottant role in transport of therapeutic agents of our invention from the olfactory nerves of the olfactory sensory organ (besides the branches of the trigeminal and sphenopalatine ganglion) for the sense of smell located at the medial and lateral wall of the nose (FIGS. 1, 2, 3) extending above the level of middle turbinate and occupies the upper third of the nasal cavity. The olfactory nerves, bundled ±20 in number, formed from the axons of the olfactory sensory neurons surrounded by perineural epithelium and sub perineural epithelial space (FIGS. 4, 5, 6, 7, 12, 13) from the olfactory mucosa which pass through the cribriform plate of the ethmoid bone to the olfactory bulb where interconnections and synapses occur. (FIG. 7) From the olfactory bulb, olfactory tract reaches, gets connected and interconnected to the olfactory area in the temporal lobe of the cerebral cortex in each hemisphere, hypothalamic area, median eminence and other brain stem nuclei. (FIG. 9, 10) The ophthalmic and maxillary branches of the trigeminal nerves and sphenopalatine ganglion are located very close to olfactory mucosa in front and back of olfactory mucosa. (FIGS. 3, 4, 8, and 9). They get connected to the basal part of the brain stem delivering therapeutic agents to this region. Anti autism therapeutic agents (insulin, IGF-1 and other drugs) deposited in the olfactory mucosal region (ORE) reach the CNS centers in the treatment of autism through these neuronal and vascular networks connected to the CNS centers.

Delivery of Therapeutic Agents to the CNS Through the Olfactory Nasal Pathways (ORE) to the Brain in Our Invention to Treat Autism

The Olfactory Nerve And Olfactory Mucosal Pathway Includes for the delivery of therapeutic agents in our invention includes the he Following Components:

1. Olfactory mucosa with ±20 bundles of axonal nerve fasciculi being formed below the cribriform plate of the ethmoid bone by the olfactory receptors axons, surrounded by the perineural epithelial covering (FIG. 6) and enter though the perforated ethmoid bone to the undersurface of the olfactory bulb and get connected.

2. Anterior ethmoidal nerves from the ophthalmic branch of the trigeminal nerve located in front of the olfactory mucosa.

3. Sphenopalatine ganglion (Synonyms: Pterygopalatine ganglion, pterygopalatinum, Meckel's ganglion, nasal ganglion) and it branches (FIG. 2) especially medial and superior nasal nerves and nasopalatine nerves; various sympathetic and parasympathetic connections with this neuronal ganglion, plexus around the blood vessels and their connection with the CNS. The sphenopalatine ganglion (PG) has extensive peripheral and central connections (FIGS. 2, 3) through which the ORE therapeutic agents can spread. Besides therapeutic agents passing directly to the brain through the ORE, the therapeutic agents used to treat autism can spread from the sphenopalatine ganglion through the sensory, motor, parasympathetic and sympathetic roots that it is connected to form the ganglion. The possible roots involved in therapeutic agents spreads both centripetally and centrifugally are: sympathetic efferent (postganglionic) fibers from the superior cervical ganglion travel through the carotid plexus, and through the deep petrosal nerve. The deep petrosal nerve joins with the greater petrosal nerve to form the nerve of the pterygoid canal, which enters the sphenopalatine ganglion. Its sensory root is derived from two sphenopalatine branches of the maxillary nerve; their fibers pass directly into the palatine nerves and the motor and the parasympathetic root. Its motor root is derived from the nervus intermedius (a part of the facial nerve) through the greater petrosal nerve (parasympathetic). From this complex ganglion, the therapeutic agents spread through the branches which supply the nose, soft palate, tonsils, uvula, roof of the mouth, upper lip and gums, and to the upper part of the pharynx and the carotid artery they surround. From this region, the anti-autism therapeutic agents are carried to the front part of the brain stem. The lacrimal gland is also connected by the sphenopalatine ganglion via the zygomatic nerve, a branch of the maxillary nerve (from the trigeminal nerve) connects with the lacrimal nerve (a branch of the ophthalmic nerve which is part of the trigeminal nerve) to arrive at the lacrimal gland.

When we refer ORE, it includes olfactory mucosa (OM), the olfactory nerves, anterior ethmoidal nerves from the ophthalmic branch of the trigeminal nerve, and all the nerves connected with sphenopalatine ganglion including the ophthalmic and maxillary nerve, complex network of communicating blood vessels between this region and inside the cranium; though the major pathway is being olfactory mucosa, olfactory nerves and the olfactory bulb and the sub arachnoid space (SAS) around the olfactory bulb and CSF within it. The therapeutic agents in our invention reach the CNS through these routes in the treatment ASD.

Nasal Blood Vessels Delivery of the Insulin and Therapeutic Agents to the CNS Includes the Following Vascular Systems and their Connections.

The blood vessel (BV) uptake and distributions of the intranasal administered drugs to the CNS can be divided into three components.

1. The BV of the respiratory mucosa of the nose absorbs the therapeutic agents and distributes all over the body through systemic circulation including the nervous system. This is route is selected to deliver therapeutic agents to treat local (anti-allergy therapeutic agents) and systemic diseases and plays hardly any role in the spread of insulin and other therapeutic agents to the CNS in the treatment of ASD.

2. The BV's of the olfactory mucosa and ORE are intimately associated with the above described neuronal components and carry the therapeutic agents directing through their connection to the subarachnoid space and to the CNS. They have complex connections between the nose and the brain especially to the hypothalamic area, pituitary, median eminence, arcuate nucleus, cavernous sinus, front part of the temporal lobe, undersurface of the frontal lobe, and other areas of the brain stem nuclei, SAS and CSF. The eyes are also connected to the CNS through the vascular route through these blood vessel networks, arachnoid villi and SAS. They all play an important role in transport of therapeutic agents to CNS for the treatment of ASD.

3. The therapeutic agents from the BV, especially from the respiratory and olfactory mucosal part of the nose, enter the systemic circulation and then enter the brain and leak the therapeutic agents into ventricles through the circumventricular organs, choroid plexus, leptomeninges covering of the brain, and ependymal lining. The BV of these organs does not have rigid blood brain barrier (BBB) as seen in those which enter the brain and spinal cord from systemic circulation. The location of circumventricular organs in the brain (FIG. 15) thorough which the therapeutic agents can enter the CNS are: median eminence, area postrema, organum vasculosum of the lamina terminalis, pineal gland, posterior pituitary, subformical organ as well as choroid plexus and ependymal lining of the ventricles and central canal of the spinal cord (Modified from Saper and Breder, New England journal of Medicine 330: 1080-1886, 1994.). From these regions, the therapeutic agents reach the CSF which circulates through the neuropil and exert their therapeutic effect in the treatment of autism.

The therapeutic agents of our invention to treat ASD leak into CSF through the sub perineural epithelium space of the nerve fasciculi of the olfactory nerves (FIGS. 5, 7), from trigeminal and sphenopalatine ganglion nerves from the olfactory mucosal surface to the olfactory bulb and then spread to the SAS which reach the cerebral cortex and brain stem nuclei through the cerebrospinal fluid (CSF) of the subarachnoid space (SAS).

Main Route of Spread of Therapeutic Agents from the Olfactory Mucosal Surface Through the Axons, Perineural Epthilium, Subperineural Space Connected to the Subarachnoid Space (SAS)Virchow-Robins Space, and CSF then to the Neuropil

It has been shown that the spread through the axon of the olfactory receptors, and trigeminal verve complexes takes days and is very slow. The therapeutic agents deposited on olfactory mucosal and ORE area to treat autism in our invention reaches rapidly through the following routs and axons play a minor role in the spread of therapeutic agents.

1. The therapeutic agents on the olfactory mucosa itself spread to CNS as follows. The therapeutic agents are attaches to the mucus lining of the olfactory nerves. It passes to the sensitive bipolar olfactory cells, axons and transported through axons to the olfactory bulb synapses and the rest of the CNS. The therapeutic agents have hard time passing through the complex synaptic system of the olfactory bulb (Glomeruli) to reach the final destination to the CNS nuclei. That is why the olfactory axonal spread of therapeutic agents takes days and plays a minor role in the treatment of autism in our invention.

2. The sustenticular (supporting) cells between the olfactory receptor cells have hundreds of microvilli, which pinocytose the therapeutic agents inside the cells. The therapeutic agents also leak between the receptors cells and sustenticular cells. From here, the therapeutic agents enter the subperineural epithelial space which surround the olfactory nerve bundles and reaches the SAS and CSF surrounding the olfactory bulb to reach the CNS neuropile. (FIGS. 4-7)

3. The therapeutic agents enter the olfactory mucosa between the sustenticular and receptor cells and the space left by the dying or dead olfactory neurons. Then pass below the basal cells and reach the subperineural space which surrounds the axonal nerve bundles. (FIGS. 4-7; T. R. Shantha, Virchow-Robin space in the peripheral nerves, 1992, ASRA March-April Supplement). This space is openly connected to the SAS of the olfactory bulb SAS, and directly communicates with the CSF fluid in the SAS surrounding the olfactory bulb and CSF of the CNS close to the optic chaisma, median eminence, pituitary stalk, optic chaismal CSF cistern, pontine cistern, anterior surface of the brain stem and other structures in the vicinity. Once the therapeutic agents are in this space, they have easy route or accesses to the SAS of the CNS and reach the neuropile. Hence, from the SAS from the olfactory bulb, therapeutic agents easily spread to the cortical areas, hypothalamus, thalamus and other nuclei in the brain stem. From CSF, the insulin, IGF-1, and therapeutic agents to treat autism enters the perforating BV from the SAS to the neuropil. The therapeutic agents in the CSF surrounding the optic chaisma, pituitary stalk, median eminence and pontine cistern enter the neurons concerned with the autism in the cerebral cortex and brain stem nuclei.

4. The therapeutic agents spread through the Bowman's gland is minimal.

5. Spread through the trigeminal, sympathetic, parasympathetic nerves of the peripheral nerves (sphenopalatine ganglion) takes place by two mechanisms.

a. The blood vessels surrounding the nerve bindles (fasciculi) and in the lamina propria of the olfactory mucosa carry the therapeutic agents through the Virchow-Robin space inside the nerve fasciculi. (FIGS. 12, 13. T. R. Shantha IBID). The therapeutic agents also enter the nerve fasciculi by simple diffusion through the perineural epithelium covering. Once the drug enters the subperineural space around the nerve fasciculi, it is carried to the CNS through its direct communication to SAS and CSF.

b. From the subperineural space of the nerve fasciculi, the therapeutic agents permeate to the interstitial spaces of axons; enter the axons and axoplasm only through the node of Ranvier (FIGS. 12, 13, 14). The myelin sheath is a formidable barrier for the entry of therapeutic agents but not the node of Ranvier. The axoplasm has both antegrade and retrograde flow. Hence, from the axoplasm it can reach the neuropil and nuclei which they are connected to these cranial nerve roots. This method of spreads takes days and only a small fraction of therapeutic agents are transported through this route.

The neural connections between the olfactory region of the nose and the brain provide a unique pathway for the non-invasive delivery of various known therapeutic agents, as well as other pharmaceutical, biochemical, nurticeuticals, and biological agents or compounds to the CNS to treat autism. For olfactory mucosal delivery the following classes of therapeutics have successfully been intranasally delivered to the CNS such as: neurotrophins (NGF), insulin, insulin-like growth factor [IGF-1]; neuropeptides (hypocretin-1); and exendin; cytokines (interferon β-1b) and erythropoietin; polynucleotide's (DNA plasmids and genes; and small molecules like chemotherapeutics and carbamazepine. ORE delivery works best for potent therapeutics that are active in the nanomolar range. Even therapeutics that is substrates for the P-glycoprotein efflux transporter, which is known to operate in the nasal epithelium, have been reported to reach the CNS in effective concentrations. (Leah R Hanson and William H Frey II Intranasal delivery bypasses the blood-brain barrier to target therapeutic agents to the central nervous system and treat neurodegenerative disease. This article is available from BMC Neuroscience: biomedcentral.com/1471-2202/9/S3/S5. Han I K, Kim M Y, Byun H M, Hwang T S, Kim J M, Hwang K W, Park T G, Jung W W, Chun T, Jeong G J, Oh Y K: Enhanced brain targeting efficiency of intranasally administered plasmid DNA: an alternative route for brain gene therapy. J Mol Med 2006, 85:75-83. Thorne R G, Frey W H 2nd. Delivery of neurotrophic factors to the central nervous system: Pharmacokinetic considerations. Clin Pharmacokinet 2001; 40: 907-946). The olfactory neural and ORE pathway provides both an intraneuronal and extraneuronal pathway into the brain. (Shantha IBID; Thorne R G, Emory C R, Ala T A, Frey W H 2nd. Quantitative analysis of the olfactory pathway for drug delivery to brain. Brain Res 1995; 692:278-82.28. Balin B J, Broadwell R D, Salcman M, el-Kalliny M. Avenues for entry of peripherally administered protein to the central nervous system in mouse, rat, squirrel and monkey. J Comp Neurol 1986; 251:260-80. Broadwell R D, Balin B J. Endocytic and exocytic pathways of the neuronal secretory process and trans-synaptic transfer of wheat germ agglutinin-horseradish peroxidase in vivo. J Comp Neurol 1985; 242:632-50).

The intraneuronal pathway involves axonal transport and requires days for therapeutic agents to reach different regions of the brain. While the extraneuronal pathway relies on bulk flow transport of therapeutic agents in the treatment of autism through sub perinueral epithelial channels as described above, the drugs are delivered directly to the brain parenchymal tissue through CSF. The extra neuronal pathway we describe in various publications (perineural epithelium and subperineural spaces and their connection to SAS and CSF) allows therapeutic agents to reach the CNS within minutes of administration (Frey W H, Liu J, Thorne R G, Rahman Y E. Intranasal delivery of 125I-labeled nerve growth factor to the brain via the olfactory route. In: Iqbal K, Mortimer J A, Winblad B, Wisniewski H M, editors. Research advances in Alzheimer's disease and related disorders. New York: John Wiley and Sons Ltd; 1995. p. 329-35. Frey W H, Liu j, Chen X, Thorne R G, Fawcett J R, Ala T A. Delivery of 125I-NGF to the brain via the olfactory route. Drug Delivery 1997; 4:87-92. Chen X Q, Fawcett J R, Rahman Y E, Ala T A, Frey W H. Delivery of nerve growth factor to the brain via the olfactory pathway. Alzheimers Dis 1998; 1:35-44. Frey W H, Thorne R G, Pronk G. Delivery of Insulin like growth factor-1 to the brain and spinal cord along olfactory and trigeminal pathways following intranasal administration: a noninvasive method for bypassing the blood brain barrier. Soc Neurosci Abstract 2000; 26:1365-70.30-33).

Olfactory mucosal and ORE intranasal delivery of therapeutic agents in our invention enter the CSF. It is not surprising as CSF normally drains along the olfactory nerve fasciculi as they traverse the cribriform plate of the ethmoid bone and approach the olfactory submucosa in the roof of the nasal cavity, where the CSF is then diverted into the venous system and nasal lymphatics. It is important to note that like neuropil of the CNS, neurons in the olfactory mucosa are also kept immersed in the CSF from the olfactory bulb SAS which conduct the therapeutic agents to the CNS in the treatment of ASD. The lymphatics hardly play any role in transport of therapeutic agents retrograde to the CNS neurological tissue. The olfactory mucosa and the olfactory nerves are completely soaked in the CSF from the brain through the sub perineural epithelium spaces described by Dr. Shantha in multiple publications (Shantha T R and Bourne G H: The Perineural epithelium: and significance. J Nature 199, 4893:577-579 (1963). Shantha T R and Bourne G H: Perineural epithelium: A new concept of its role in the integrity of the peripheral nervous system. Science 154:1464-1467 (1966). Shantha. T. R, and Bourne. G. H, Perineural Epithelium, in G H Bourne, Ed. In Structure and Function of Nervous Tissues. Volume I. Academic Press, New York. 1968. Prophecies and predictions 379-459; Shantha and Nakajima. Histological and Histochemical Studies on the Rhesus Monkey (Macaca Mulatta) Olfactory Mucosa. Z. Zellforsch. 103, 291-319 (1970). Thorne, et al., have reported that the trigeminal neural pathway may also be involved in rapidly delivering protein therapeutic agents, such as insulin-like growth factor-1 to the brain and spinal cord following intranasal administration (Thorne R G, Emory C R, Ala T A, and Frey W H 2nd. Quantitative analysis of the olfactory pathway for drug delivery to brain. Brain Res 1995; 692:278-82). We believe that the sphenopalatine ganglion and the trigeminal nerves which connect and innervate the nasal mucosa and the CNS also play a role in transport of therapeutic agents from the nose to the CNS through the SAS, CSF, BV and Virchow-Robins space and sub perineural epithelial space especially to the brain stem region of CNS. The transport by these nerve structures is slower compared to the dozens of olfactory nerve fasciculi delivery to the SAS of the olfactory bulb, to the CSF and to the CNS.

Therapeutic Agents Transfer from CSF to CNS by Tanacytes

The median eminence, arcuate nucleus, circumventricular organs, and ependymal linings of the ventricles and central canal of the spinal cord contain a population of specialized ependymal cells, called tanycytes. (FIG. 20) Tanycytes are bipolar cells bridging the cerebrospinal fluid (CSF) to the portal capillaries and may link the CSF to neuroendocrine events and neuropil. They are most numerous in the third ventral of the brain, but can also be seen in the spinal cord radiating from the ependyma of the central canal to the spinal cord surface. It is possible that their function is to transfer chemical signals from CSF to CNS vice versa and transport of neuropil interstitial fluids back and forth. Besides the surface transport through the circumventricular organs and tanycytes, the perforating blood vessels transport the therapeutic agents deep into the neuropil close to the nuclear masses deep in the brain and exert their effect in the treatment of ASD.

Olfactory Mucosal Mechanisms of Absorption of Insulin IGF-1 and Therapeutic Agents in Treatment of ASD in Our Invention:

The therapeutic agents from olfactory mucosal area are absorbed fast depending upon lipophilicity; and at a slower rate depending on molecular weight. Lipophilic substances in the form of micelles can be added to the therapeutic agent's composition to enhance absorption of the pharmaceutical agent across the ORE. The research data indicate that good bioavailabilities can be achieved for molecules up to 1000 Da (without enhancers) and good availability can be extended to at least 6000 Da with uptake absorption enhancers. Dalton is a unit of mass very nearly equal to that of a hydrogen atom. Dalton is named after John Dalton (1766-1844), who developed the atomic theory of matter. A Kilodalton kD is a unit of mass equal to 1000 daltons. Our studies show that the insulin, a protein with a molecular weight of 40900 has a mass of or 40.9 kD (Sjogren, B., and Svedberg, T., J. Am. Chem. Sot., 63, 2657 (1931) 35 kD. Polson, A., Kolloid-Z., 87, 149 (1939) 40.9 KD). This large molecule of insulin easily permeates the olfactory mucosa, and enters the CSF of SAS; indicting, therapeutic agents with higher molecular weight can be administered intranasally to treat ASD and other neurodegenerative diseases such as Alzheimer's, Parkinson's etc. Sakane et al. (Sakane T, Akizuki M, Yamashita S, Nadai T, Hashida M, Sezaki H. The transport of cephalexin to the cerebrospinal fluid directly from the nasal cavity. Pharm Pharmacol 1991; 43: 449-451) reported that following intranasal administration of the antibiotic Cephalexin (34.739 kD) to rats, 166-fold higher compared to systemic administration. Cephalexin hardly crosses the BBB and it was concluded that Cephalexin entered the CSF directly from the nasal cavity through ORE. Using a series of fluorescein isothiocyanate-labeled dextrans (FITC-dextran) it was found that dextrans with molecular weights of up to 20,000 daltons be transported directly from the nasal cavity of rats into the CSF. These FITC-dextrans were not found in the CSF after intravenous administration due to impenetrable BBB.

The transport mechanisms of different substances like insulin, mannitol and propranolol across the nasal mucosal tissue were studied by Wheatly, et al. (Wheatly M A, Dent.), Wheeldon E B, Smith P L. Nasal drug delivery: An in vitro characterization of transepithelial electrical properties and fluxes in the presence or absence of enhancer. J Control Release 1988; 8:167-77). The transport of these substances occurs by a passive transport mechanism through the sub perineural epithelial space connected to the SAS and CSF as described in our publications. The transport of tyrosine and phenylalanine across rat mucosa were absorbed by an active saturable transport process, which appeared to be Na+ dependent, and transport may have required metabolic energy as a driving force. Transport of insulin through the ORE is reported in numerous publications and in our studies, and is ideally suited in the treatment of ASD. Insulin also augments and amplifies the uptake by the tissues and effects of other therapeutic agents in the treatment.

Drug Absorption and Transport from Olfactory Mucosa

The initial phase in the absorption of drugs from the nasal cavity and the olfactory mucosal passage (ORE) is through the mucus. Mucin, the principal protein in the mucus, binds solutes, hindering diffusion. After a drug's passage through the mucus, mechanisms for absorption the mucosa include: 1. transcellular or simple diffusion across the membrane, 2. paracellular transport via movement between cells, and 3. transcytosis by vesicle carriers. Of course, the nasal absorption is affected by molecular weight, size, formulation, pH, pKa of molecule, and delivery volume among others. Hydrophilicity has been found to decrease a drug's bioavailability and pH is also an important formulation factor for drug absorption and distribution. Both the pH of the nasal cavity and pKa of a particular drug need to be considered to optimize absorption. Nasal irritation is minimized when products are delivered with a pH range of 4.5 to 6.5.50. Volume and concentrations are also important considerations (Romeo V D, De Meireles J, Sileno A P, Pimplaskar H K, Behl C R. Effects of physiochemical properties and other factors on systemic nasal drug delivery. Adv Drug Del Rev 1998; 29:89-116). We have used 1.5 to 3 ml per ORE area in each nose in our studies using a special delivery catheter (see FIG. 8, 8a, 9) to deposit the therapeutic agents close to the olfactory mucosal and ORE area with minimal spread to the respiratory mucosa of the nose.

The BBB and Delivery of Therapeutic Agents to CNS from the Olfactory Region (ORE) in the Treatment of Autism in Our Invention

The blood-brain barrier (BBB) is an innate barrier or strict gate keeper protecting the CNS (brain) from damage by injurious substances that enter the blood stream. Intranasal ORE administration of insulin, IGF-1, as well as various known therapeutic agents circumvents the BBB to be transported rapidly and directly to the CNS. The olfactory region has exceptional anatomic, histologic and physiologic attributes that provide both extracellular and intracellular pathways into the CNS that bypass the BBB through the olfactory mucosal surface, olfactory neurons, trigeminal nerves, autonomic ganglions, and interconnecting blood vessels. We believe that the lymphatic play hardly any role in the transport of ORE delivered insulin and therapeutic agents. It is important to note that the olfactory sensory neurons are the only first order neurons whose cell bodies are located in a distal epithelium in the upper part of the nose. Their dendrites are directly exposed to the external environment through the mucous coating of the olfactory mucosa (FIGS. 4, 5) of the upper nasal passage; while their axons project (FIGS. 4, 5, 6) through perforations in the cribriform plate of the ethmoid bone as bundles of nerve fasciculi (±20 of them) surrounded by perineural epithelium, pass through the ethmoid bone to synaptic glomeruli in the olfactory bulb (FIGS. 4-7).

To the therapeutic agents preparations with insulin and other therapeutic agents; other absorption facilitators and enhancers which break open the tight junctions of the olfactory mucosa, disrupt the membranes, break down the mucous layer (mucolytic), reduce clearance and inhibit the enzyme activity can be added. They are in the form of bile salts and derivatives (Sodium deoxycholate, sodium glycocholate, sodium taurodihydrofusidate); Surfactants' (Sodium lauryl sulphate, saponin, polyoxyethylene-9-lauryl ether); Fatty acids (Sodium caprylate, sodium laurate, phospholipids e.g., Cyclodextrins; dideconoylphosphatidylcholine, lysophosphatidylcholine); and Bioadhesive materials (Powders Carbopol, starch microspheres; and Liquid Chitosan, and carbopol).

Insulin, IGF-1, and various known therapeutic agents used in the treatment of autism, has to pass through the axons of the olfactory mucosal cells and be transported through the complex synaptic stations such as Glomeruli, periglomerular cells, mitral cells, and granule cells of the olfactory bulb (FIG. 7), then, has to pass through the medial and lateral olfactory tact's to reach the CNS. Such mechanism of transport of therapeutic agents does take place, but will take days to reach the final desired destination through this route. Our studies (unpublished) and other studies have demonstrated that intranasal ORE administration provides direct access to IGF-1, insulin and other adjuvant pharmaceutical, biochemical, nurticeuticals, and biological agents or compounds to be transported to the CSF surrounding the olfactory bulb from the sub perineural epithelial and interstitial space of the olfactory nerve fasciculi within ±30 min without substantial uptake into the bloodstream (FIGS. 4-7). Given that intraneuronal, axonal transport requires days to transport the substances and to reach the brain, that intranasal ORE instilled insulin and therapeutic agents passing through intercellular crevices, fissure, and gap between the normal, budding and dying olfactory epithelial sensory receptors (FIGS. 4, 5), supporting cells and basal cells; diffuses into the sub perineural epithelium and interstitial spaces in the nerve fasciculi below the sub perineural epithelium space surrounding the group of olfactory fasciculi and is rapidly delivered to sub arachnoid space to CSF of olfactory bulb. From this SAS, the therapeutic agents are distributed to the rest of the neuropil as shown in FIGS. 7. 10. From the sub arachnoid space CSF, the various known therapeutic agents are carried to the neurons and nuclei through the pia mater and Virchow robin space (FIG. 16) as well as blood vessels. In addition to the olfactory pathway, insulin, and insulin-like growth factor-I and other therapeutics have also been shown to be transported from the nasal olfactory mucosa to the brain along the trigeminal neural pathway to achieve maximum immediate therapeutic effect in the treatment of autism as described below.

Insulin, Insulin-Like Growth Factor-I (IGF-1) to Treat Autism and Role Insulin Plays with the Uptake, Distribution; Augmentation—Amplification Effects of Therapeutic, Pharmaceutical, Biochemical and Biological Agents or Compounds in the Treatment of Autism Using Our Invention are Described Herein.

A variety of carriers, adjuvant agents, absorption enhancers, and facilitators, assist to get entry into the cell, potentiators of therapeutic action (augmentation/amplification effects), cell metabolic activity enhancers, cell multiplication enhancers, and other methods have been used to enhance the absorption and/or to potentiate the effect of therapeutic, pharmaceutical, biochemical, and biological agents or compounds administered to the patients for improving the physiological function, and the treatment of diseases. The discovery of insulin, as described in this invention is such a biological agent which will cure or curtail autism and at the same time augment and amplify the effects of other therapeutic agents. The following describes insulin and its biological effects in the treatment of autism.

Besides aspirin and antibiotics, insulin is the most commonly used therapeutic agent known to the public and professional alike. Insulin is a hormone secreted by beta cells of the islets of Langerhans in the pancreas. It has been self-administered in home by the patient or in the office by the physician to treat diabetes. Insulin can be easily obtained by prescription, and insulin can be used for treating autism as described in this invention. So far, there are no reports of using the insulin as a therapeutic agent to treat localized diseases or parentarily to treat systemic diseases other than diabetes. The present inventor is the first person to experiment with the use of insulin locally for almost a decade to treat many kinds of diseases of various tissues and organs in the body with many known adjuvant therapeutic, pharmaceutical, biochemical, and biological agents or compounds; and systemically to treat Alzheimer's, Parkinson's, autism, depression and many neurodegenerative diseases.

In 1965, Sodi-Pollares, et al., used glucose-insulin-potassium (GIK) solutions for the first time to treat patients with acute myocardial infarction. He found that GIK limited infarct size, reduced ventricular ectopy, and improved survival (Sodi-Pollares D, Testelli M D, Fisleder B L. Effects of an intravenous infusion of a potassium-glucose-insulin solution on the electrocardiographic signs of myocardial infarction. Am J Cardiol. 1965; 5:166-81). Insulin benefits the post ischemic myocardium by stimulating pyruvate dehydrogenase activity, which activity, in turn, stimulates aerobic metabolism on cardiac and other tissue reperfused.

Insulin added to antegrade and retrograde tepid (29° C.) blood cardioplegia during coronary artery surgery has been shown to decrease the levels of free fatty acids, increase myocardial uptake of glucose, stimulate aerobic metabolism during reperfusion, preventing lactate release and improving left ventricular stroke work index with the restarting of the heart beating without many arrhythmias (Svensson S, Svedjeholm R, Ekroth R. Trauma metabolism of the heart: uptake of substrates and effects of insulin early after cardiac operations. J Thorac Cardiovasc Surg. 1990; 99:1063-73. Rao V, Mississauga C N, Merrante F. Insulin cardioplegia for coronary bypass surgery [abstract]. Circulation. 1998; 98 (Suppl): 1-612). Insulin increases the glutathione synthesis by activating gamma-glutamyl-cysteine synthetase thus increasing intracellular glutathione (GSH) content in oxidized cells. This effect will help in the treatment of autism. The results show that GSH can reverse the effect of oxidation (oxidative free radical damage) on tyrosine kinase activation and phosphorylation. Thus, GSH plays an important role in cell signaling, which confirms the antioxidant activity of insulin to prevent the neuropil damage by ROS and restore the brain function back to normal in autism patients. Insulin improves cellular physiological function. In addition, insulin augments/amplifies the effects of therapeutic agents. Hence, our invention, with local use of insulin alone or with other therapeutic agents, is very effective in treating autism and related afflictions of the CNS.

Insulin affects the DNA, RNA, and protein synthesis which results in increased growth by mitosis (Osborne C K, et al. Hormone responsive human breast cancer in long-term tissue culture: effect of insulin. Proc Natl Acad Sci USA. 1976; 73: 4536-4540); enhances the synaptogenesis (needed in autism patients), increases permeability of cell membranes to many therapeutic agents besides glucose, and electrolytes; insulin helps facilitate moving the drugs and therapeutic agent molecules from extra cellular fluid (ECF) to intracellular fluid (ICE) meaning from outside the cells to inside the cells. This action of insulin of our invention will facilitate the entry of other adjuvant therapeutic agents into neuropil and help in restoring the function of the brain at the neuronal and synaptic level in ASD.

Insulin and IGF-1 s have tissue growth factors properties; in addition, they have well recognized functions as hormones which regulate growth and energy metabolism at the whole organism level farther away from the site of production (insulin from the islets of pancreas, IGF-1 from the liver). Insulin and IGF-1 s differ from many other regulatory peptides in that the peptides are relevant to regulate physiology at both the whole organism level and the cellular level. They are absorbed and circulated farther away from the site of application and exert their therapeutic effects on the entire CNS (Michael Pollak. Insulin and Insulin-Like Growth Factor Signalling in Neoplasia. Nat Rev Cancer. 2008; 8(12):915-928). Besides, insulin and the IGFs produce important autocrine, paracrine, or endocrine growth factor effects. These factors will help to maintain the integrity of the neurons and their synapses in the CNS in autism patients when the insulin is transported to the brain and spinal cord from the ORE in the treatment of autism in our invention.

Benefits from the insulin transport process previously described in our invention include: Increased cellular metabolic activity induced by insulin and IGF-1, enhances the uptake; augment and amplify the effects the action of all adjuvant therapeutic, pharmaceutical, biochemical, and biological agents or compounds by the cells and inside the cell including the cells responsible or involved in autism (neurons and the synapses). Once inside the cells; the insulin augments and amplifies the effects of any and all adjuvant therapeutic agents including the agent proven and/or approved to treat autism restoring their physiological function of the neurons and their synapses in autism.

The augmentation and amplification effects of insulin on other therapeutic agents have been meticulously and conclusively demonstrated in ingenious vitro studies by Alabaster, et al. They demonstrated that insulin activates and modifies metabolic pathways in MCF-7 human breast cancer cells. The insulin increases the cytotoxic effect of methotrexate up to 10,000 (ten thousand) fold (Oliver Alabaster, et al. Metabolic Modification by Insulin Enhances Methotrexate Cytotoxicity in MCF-7 Human Breast Cancer Cells, Eur J Cancer Clinic; 1981, Vol 17, pp 1223-1228. Richard L. Schilsky and Frederick. S. Ordway. Insulin effects on methotrexate polyglutamate synthesis and enzyme binding in cultured human breast cancer cells. Cancer Chemother Pharmacol (1985) 15: 272-277). My own research studies, on every kind of cancer and infection in any part of the body, have shown that the group treated with insulin, plus with low dose anticancer agents (and/or antibiotics for infection, autoimmune diseases treatments, monoclonal antibody treatment, etc.) responded better than the patient treated with insulin or chemotherapy alone. These observations supports the findings of Alabastor (IBID); that the disease cell sensitivity to the therapeutic and biological agents as those to be used to treat autism is augmented and amplified many times by using the method described in this invention using insulin and/or IGF-I with other anti-autism therapeutic agents to treat autism.

The effect of insulin in reducing the ROS and other etiological factors in autism is profound. In an important experiment, Zheng et. al., showed the role of insulin-like growth factor-I (IGF-I) induced the inner ear epithelial cell culture growth (Zheng, J. L., Helbig, C. & Gao, W-Q. J. Neurosci. 17:216-226 (1997. There is a clear indication that insulin and IGF-I not only played a role in potentiation (augmentation/amplification effects) of the therapeutic, pharmaceutical, biochemical and biological agents or compounds when used to treat autism through their application through the ear to reach the auditory part of the nervous system involved in the autism. (Shantha T. R., Unknown Health Risks of Inhaled Insulin. Life Extension, September 2007 pages 74-79, Post publication comments in September 2008 issue of Life Extension, Pages 24. Shantha T. R and Jessica G. Inhalation Insulin, Oral and Nasal Insulin Sprays for Diabetics: Panacea or Evolving Future Health Disaster. Part I: Townsend Letter Journal: Issue #305, December 2008 pages: 94-98; Part II: Townsend Letter, January, 2009, Issue #306, pages—106-110)

Craft, et al. reported on Intranasal Insulin Therapy for Alzheimer Disease and Amnestic Mild Cognitive Impairment; published in Arch Neurol. Published online Sep. 12, 2011. doi:10.1001/archives of Neurology, We have used insulin intranasally almost a decade back to treat Alzheimer's, PTSD, Parkinson's, Lyme disease, depression, stroke and other mental conditions, etc. Insulin has a significant numbers of functions in the central nervous system as it does in the rest of the tissues of the body. Abundant insulin receptors are localized in the hippocampus, the medial temporal cortex (area 24, 38), hypothalamus, and the frontal cortex of the CNS which are involved in the ASD and the neurodegenerative diseases. The insulin receptors are found mainly in synapses, where insulin signaling contributes to synaptogenesis, regulates synapse number, synaptic remodeling and dendritic plasticity (Chiu S L, Chen C M, Cline H T. Insulin receptor signaling regulates synapse number, dendritic plasticity, and circuit function in vivo. Neuron. 2008; 58(5):708-719. Zhao W Q, Townsend M. Insulin resistance and amyloidogenesis as common molecular foundation for type 2 diabetes and Alzheimer's disease. Biochim Biophys Acta. 2009; 1792(5):482-496.), which very much needed in the treatment of ASD.

The importance of insulin in normal brain function is underscored by evidence that insulin dysregulation contributes to the pathophysiology of autism and other neurodegenerative diseases, which are the disorder with synaptic delay and characterized in its earliest stages by synaptic loss. Insulin levels and insulin activity in the central nervous system are reduced in AD, and possibly in autism. Hence, the reduced levels of insulin and of insulin activity contribute to a number of pathological processes that illustrate and typify autism. Our invention corrects this pathological process in the treatment of ASD.

Thus, our studies also found that restoring insulin to normal levels in the brain provide therapeutic benefit with, autism (so also in AD, mental patients Parkinson's, depression, PTSD, depression, Lyme disease, CNS effects, stroke, MS, ALS patients in our studies; the list is endless). Peripheral parenteral administration of insulin is not advocated because of its hypoglycemic effects. On the contrary and as explained above, intranasal ORE (not to respiratory part of nasal mucosa) administration of insulin provides rapid delivery of insulin to the central nervous system via olfactory and trigeminal neural pathways (FIGS. 1-16) without affecting blood insulin or glucose levels in the treatment of autism.

We used intranasal insulin and glucose, which improved the memory even faster and reduced the symptoms of autism and restored the speech as seen in stroke. Studies show that the administration of intranasal insulin stabilized or improved cognition and function and preserved changes in the cerebral metabolic rate of glucose (CMRGIc) utilization assessed by use of positron emission tomography (PET) with fludeoxyglucose F 18 (FDG) in regions affected by AD.

There are various forms of insulin used to treat diabetes; these different forms of insulin can be formulated to be used in this invention. They are grouped under rapid, short, intermediate, and long-acting insulin. The insulin is dispensed as premixed form containing rapid to long acting insulin. Insulin products are categorized according to their putative action profiles as:

1) Rapid-acting: insulin lispro, insulin aspart, and insulin glulisine

2) Short-acting: regular (soluble) insulin

3) Intermediate-acting: NPH (isophane) insulin

4) Long-acting: insulin glargine and insulin detemir

We have used rapid and short acting insulin to treat autism. The dose is anywhere from 3 IU to 5 IU per nostril to ORE in children and 10 IU in each ORE in adults mixed with saline as dilutant. In adults, the dose can be as high as 10 to 20 IU per side of the nose. The dose can be decreased or increased depending upon the age and weight of the autism patient. We have used the same dose for the ear to treat autism along with intranasal ORE delivery of insulin.

One has to realize the possibility of developing hypoglycemia when the insulin is being used at ORE and ear. Patients will be warned about the possibility of hypoglycemia where they will be prepared for a hypoglycemic reaction. IGF-I has hypoglycemic effects in humans similar to those of insulin when administered by intravenous bolus injection. In addition, single dose of rhIGF-I reduces overnight GH levels and insulin requirements in adolescents with IDDM. In our practice of using the insulin, nasally and on the external ear for decades, we never reported the development of a single case of hypoglycemic event which needed therapy. Signs and symptoms of hypoglycemia which need to be told to patient include rapid heartbeat, sweating, dizziness, confusion, unexplained fatigue, shakiness, hunger, feeling hot, difficulty in thinking, confusion, headache, maybe even develop seizures, and the potential loss of consciousness with severe hypoglycemia. Once symptoms of hypoglycemia develop, the patient should be treated with oral ingestion of a fast-acting carbohydrate such as glucose tablets, fruit juice, fruit bowl, chocolate bar, regular Coca-Cola, sugary drinks or eat plain sugar followed with a drink of water or IV administration of 25% glucose, if the reaction is severe.

Method of Administration of Insulin and Selected Therapeutic Agents Used to Treat Autism

Preparation of the Insulin Drops:

Mix 100 units of fast acting insulin with 5 ml of saline. Each ml will contain 20 IU of insulin. That means each 0.25 ml will contain 5 units of insulin. The amount of dilution of insulin preparation can be variable. Examine the patent and make sure the patient had light meal. If there is allergic nasal dripping, constrict the turbinate's with vasoconstrictor nasal spray. Clean the nose completely of all the secretions before instilling therapeutic agents. Add other adjuvant therapeutic agents into to preparation prescribed amount.

Preparation of Other Therapeutic Agents to be Used with Insulin and IGF-1

If the injectable therapeutic agents are indicated for the treatment of autism, all one has to do is dilute to the desired dose to be administered intranasal ORE by using appropriate dilutant. If it is not in an injectable form, but is, instead in solid form, the solution is prepared using normal saline, distilled water, or Tris; pH adjusted, clarified and filled into appropriate size ampoules sealed by fusion of the glass or sterile rubber cap. The solution is sterilized by heating in an autoclave using one of the acceptable cycles. Alternatively, the solution may be sterilized by micro filtration and filled into sterile ampoules under aseptic conditions. The solution may be packed under an inert atmosphere of nitrogen or other suitable gas. We have used both these methods to prepare the solution to administer intranasal ORE. The dose administered through the ORE is reduced up to 80% of parenteral dose.

Oral Anti Autism Therapeutic Agents with ORE Administration of Insulin and IGF-1 to Treat Autism

It is important to note that insulin and IGF-1 can be administered through the ORE and ear. On the other hand, various formulations further comprising adjuvant therapeutic agents may not be available or cannot be formulated to deliver through the ORE and ear. They have to be taken orally to exert their effect on the CNS. These oral drugs do have to pass the BBB to reach the neuropil and exert their effect with insulin and IGF-1. Hence, there are two routes of delivery of other adjuvant therapeutic agents other than insulin and IGF-1. They are as follows:
a) The other method is to administer the oral medication, which cross the BBB at least 60 minutes before the ORE administration of insulin and IGF-1, so that the oral medications have reached the CNS crossing the BBB to exert their desired pharmacological effect. Then, administer insulin and IGF-1 through the ORE and auditory routes. The insulin will augment and amplify the effects effect of therapeutic agents circulating in the blood of the neuropil of the CNS and spinal cord after absorption through the ORE administration. Hence the oral dose of these adjuvant therapeutic agents can be reduced drastically. Formulating the oral forms by dissolving in solvent and delivering to ORE and ear are described above.

Within consummate, ideal, exemplary embodiments; insulin, and/or IGF-1 can be administered simultaneously or sequentially, in combined or separate formulations, with one or more secondary adjuvant therapeutic agents or other indicated anti autism therapeutic agents such as: serotonin reuptake inhibitors, selective serotonin reuptake inhibitors including, but not limited to, fluoxetine, fluvoxamine, sertraline, clomipramin; antipsychotic medications including, but not limited to, haloperidol, thioridazine, fluphenazine, chlorpromazine, risperidone, olanzapine, ziprasidone; anti-convulsants, including, but not limited to, carbamazepine, lamotrigine, topiramate, valproic acid, stimulant medications including, but not limited to, methylphenidate, a2-adrenergic agonists, amantadine, and clonidine; antidepressants including, but not limited to, Naltrexone, lithium, and benzodiazepines; anti-virals, including, but not limited to valtrex; secretin; axiolytics including, but not limited to buspirone; immunotherapy agent such as Monoclonal antibodies (mAB), oxytocin, manetine, etc. Additional adjuvant therapeutic agents consist of vitamins including but not limited to, B (B6, B12, and thiamin), vitamin A and D₃, and essential fatty acids. Other therapies may include behavioral modification and changes in diet such as a gluten-casein free diet with probiotics and transfer factor.

Intranasal Ore Administration Procedure

Get the patient examined by the specialist and establish the diagnosis of ASD and its type. Make sure the patients and care givers participate during the treatment so that they can carry out the treatment at home.

Extend the patient head as far one can with occipital and neck support. Make sure one does not hyperextend too far and injure the neck. Hold the child head firmly supported by an occipital and neck support (FIG. 1a). Pass the catheter or dropper or balloon catheter as described in the diagrams (FIGS. 8,9,11) carefully after proper lubrication with KY jelly or local anesthetic lubricant jelly. Pass their tip pass the soft part of the external naris. Hold the catheter directed towards the external canthus of the eye abutting against the outer edge of the nose, directed upwards and backwards. Do not pass it horizontally where the tip will end at the respiratory mucosa; hence it will not deliver the therapeutic agents to desired ORE. Then, slowly drip or spray the insulin and other therapeutic agents in the dropper, or syringe or spray bottle, through the catheter. Keep the child head hyper extended for 3-5 minutes. Pull the drug delivery device out, slowly. Keep the child supine position with head extended on a neck support for another 10-15 minutes or more.

Given the complex unknown pathophysiology of ASD, it may be naive to hope for a single “magic bullet” that will result in neuroprotection, rescue of damaged neurons and restore CNS function to normal levels in ASD. That is why, besides insulin, multiple examples that can be used in the treatment of ASD are described below. Insulin and the IGF-1 is the common denominator and magic bullet in our in invention included in all the following examples. It is important to note, if the signs and symptoms of hypoglycemia develop, it is an indication that the insulin is delivered to respiratory part of the nasal epithelium, not to the ORE as explained herein.

If the combining insulin with other therapeutic agents in single dose form delivery system is not possible, use one therapeutic agents in one side of ORE and the other on the other side of the nose, thus both the ORE are used simultaneously. Further, the therapeutic agents can be prepared and dispensed in spray or dropper dispensers with the delivery catheter device and instructions how to use it. This will help the ASD patients and caregivers to use our inventive therapeutic agents every 2-4 hours and facilitate repeated administration depending upon the symptoms relief of ASD patients.

### Example 1

As enumerated above, the insulin has a significant numbers of functions in the central nervous system. Insulin receptors are densely localized in the hippocampus, the medial temporal cortex (area 24, 38), and the frontal cortex of the CNS. The insulin receptors are found predominantly in synapses, where insulin signaling contributes to synaptogenesis and synaptic remodeling in autism (Chiu S L, Chen C M, Cline H T. Insulin receptor signaling regulates synapse number, dendritic plasticity, and circuit function in vivo. Neuron. 2008; 58(5):708-719. Zhao W Q, Townsend M. Insulin resistance and amyloidogenesis as common molecular foundation for type 2 diabetes and Alzheimer's disease. Biochim Biophys. Acta.009; 1792(5):482-496).

We have used Chitosan spray before delivering the therapeutic agents to ORE. It is a bioadhesive material which is able to decrease the clearance of formulations from the ORE, at the same time transiently opens the tight junctions in olfactory mucosal membranes (Dodane V, Khan M A, Merwin J R. Effect of chitosan on epithelial permeability and structure. Int J Pharm 1999; 182: 21-32). This will increase the stasis of the therapeutic agents at the ORE and make them pass between the olfactory mucosal receptors cells with ease and reach high therapeutic levels in the CNS which can lead to an improved therapeutic agent's response in ASD patients.

There is dysregulation of transfer of impulses at synapses in autism patients as described above. Hence, the use of ORE insulin brings homeostasis to the neuropil and the synapses (remodeling) within it and resets the synaptic transmission like normal thus eliminating the delay in transfer of information to the neuronal centers which is said to be one of the etiologies of the autism. Any synaptic delay that was noted in the ASD patients is reduced or eliminated. Further, the insulin enhances the frontal lobe activity which is also affected in autism. Place ASD patient as described above.
I. Administer insulin 2 or 4 IU in each through the nostril to ORE (depending on the age and weight of the ASD) one or two times a day through the specially designed catheter or dropper as described above. If the results are positive, with no side effects, the dose of insulin can be raised 6 units or more or less per nostril.
II. Make sure there are no signs or symptoms of hypoglycemia.
III. Let the patient stay in the neck extended position for 15-20 minutes to allow the absorption of therapeutic agents without dripping back into the nasopharynx
IV. Thirty to sixty minutes later, subject the patients to various physical, speech and other therapies prescribed as part of the protocol.
   Some of these patients we subjected to hyperbaric therapy for 30 to 45 minutes. We found that it is very useful in the treatment of autism and other neurodegenerative diseases. This will drive the therapeutic agents from the ORE to the central pathways and produce better results. We have adopted this method, whenever it was feasible.

### Example 2

I. Place ASD patient as described above.
II. Administer insulin 4 IU with 5% glucose through the nostril to ORE one or two times a day.
III. Make sure there are no signs and symptoms of hypoglycemia.
IV. Let the patient stay in the neck extended position for 15-20 minutes to allow the absorption of therapeutic agents without dripping back into the nasopharynx.
V. Thirty to sixty minutes later, subject the patients to various physical, speech and other therapies prescribed as part of the protocol.
   Insulin with glucose in the CNS will have positive effect on the neuropil involved in the genesis of ASD.

### Example 3

Insulin-like growth factors (IGFs) have a pivotal role during nervous system development and in its functional maintenance (Isabel Varela-Nieto et al. Trophic effects of insulin-like growth factor-I (IGF-I) in the inner ear. Hearing Research, Volume 196, Issues 1-2, October 2004, Pages 19-25). IGF-I and its high affinity receptor (IGF1R) are expressed in the developing inner ear and in the postnatal cochlear and vestibular ganglia. It has been shown that the trophic support by IGF-I is essential for the early neurogenesis of the chick cochlea-vestibular ganglion (CVG) by regulating the activity and/or levels of key intracellular molecules, including lipid and protein kinases such as ceramide kinase, Akt and jun N-terminal kinase (JNK). Mice lacking IGF-I lose many auditory neurons and present increased auditory thresholds at early postnatal ages. Neuronal loss associated to IGF-I deficiency is caused by apoptosis of the auditory neurons, which presented abnormally increased levels of activated caspase-3. It is worth noting that in man, homozygous deletion of the IGF-1 gene causes sensory-neural deafness (reviewed in Rev. Endo. Met. Disord. 3 (2002) 357). Thus, the IGF-I is necessary for normal development and maintenance of the inner ear and its function and that it might have contributed to the development of some of the symptoms of ASD. Thus, the trophic actions of IGF-I in the inner ear suggest that this factor have therapeutic potential for the treatment of auditory imperfection in autism. There is a delay in response to sound. The use of IGF-1 can change it and make the autism children more response to sound (hearing). As used herein, “IGF” refers to native insulin-like growth factor-I and native insulin-like growth factor-II as well as natural variants thereof such as brain IGF, otherwise known as des (1-3) IGF-I. IGF-I has hypoglycemic effects in humans similar to those of insulin when administered by intravenous bolus injection. It is important to note that the RhIGF-I has the ability to improve insulin sensitivity. Thus the administration of both insulin and IGF-1 has synergic effect and can act like insulin and cause hypoglycemia. IGF-I naturally occurs in human body fluids, for example, blood and human cerebral spinal fluid. Although IGF-I is produced in many tissues, most circulating IGF-I is synthesized in the liver. It is being used for many systemic and neurological diseases as described in U.S. Pat. No. 6,716,586 B1.

Recombinant human insulin-like growth factor-1 (rhIGF-1) is made by Tercica Pharmaceuticals marketed as Increlex. RhIGF-1 is also known as mecasermin.
I. Place ASD patient as described above.
II. Administer insulin 4 IU in each through the nostril to ORE; wait 30 minutes for it is absorbed.
III. Turn the patient on the lateral side with one ear facing up.
IV. Instill RhIGF-1 to the external ear at a dose of 30-40 mcg/kg body weight as described in the FIG. 19 with or without insulin.
V. Wait for 30 minutes; apply a cotton swab to the ear.
VI. Then turn the patient to the other side and repeat the instillation of Insulin-like growth factor-I (IGF-1)
VII. Alternatively, administer it to each ear every other day instead of the same day. Hold the head turned to opposite side for 15 minutes for allow its absorption. Cotton ball can be soaked in IGF-1 can also be placed in the ear to allow the slow absorption.
VIII. Make sure there are no signs and symptoms of hypoglycemia. Let the patient stay in the neck extended position for 15-20 minutes to allow the absorption of therapeutic agents without dripping back into the nasopharynx.
IX. Thirty to sixty minutes later, subject the patients to various physical, speech and other therapies prescribed as part of the protocol.

### Example 4

I. Place ASD patient as described above.
II. Administer insulin 4 IU in each through the nostril to ORE; wait 15-30 minutes for it is absorbed. Instill RhIGF-1 to the ORE at a dose of 30-40 mcg/kg body weight.
III. Then turn the patient to the lateral side and administer the instillation of Insulin-like growth factor-I (IGF-1) to the external auditory meatus.
IV. Alternatively, administer it to each ear every other day instead of the same day.
V. Make sure there are no signs and symptoms of hypoglycemia. Let the patient stay in the neck extended position for 15-20 minutes to allow the absorption of therapeutic agents without dripping back into the nasopharynx.
VI. Thirty to sixty minutes later, subject the patients to various physical, speech and other therapies prescribed as part of the protocol.

### Example 5

TNF is a cytokine present in humans and other mammals. Interferons are now known to be not only an antiviral and anti-proliferative cytokine, but it is also a factor which plays an important role in normal and pathological immunity. Alpha IFN is secreted by somatic cell and leukocytes, accumulating on the membranes of cells and entering the bloodstream. It plays an important role in the inflammatory immune response including in ASD development. TNF is produced by the cleavage of a transmembrane protein which aggregate in vivo to form trimolecular complexes. These complexes then bind to receptors found on a variety of cells resulting an array of pro-inflammatory effects, together with release of other pro-inflammatory cytokines, such as IL-6, IL-8, and IL-I; let loose of matrix Metalloproteinases; and up regulation of the expression of endothelial adhesion molecules, further amplifying the inflammatory and immune cascade. Etanercept (Enbrel®, Amgenllmmnunex), golimumab, infliximab (Remicade®, Centocor), adalimumab (Humira®, Abbott), COP 870, and COP 870, and onercept are in clinical development to attack these TNF. Etanercept, adalimumab, and infliximab are FDA approved for chronic systemic use to treat rheumatoid arthritis and other chronic inflammatory autoimmune diseases to counter the adverse effect of these cytokines (US 2007/0196375 AI).

ASD are considered as a disease comprising abnormalities in brain structure and/or function. Studies have suggested that ASD may have an autoimmune component (Warren et al., 1996, Mol. Chern. Neuropathol. 28: 77-81). Specifically, autoantibodies to myelin basic protein and unique antibodies to an antigenic portion of the measles component of the measles mumps-rubella (MMR) vaccine have been found in the central nervous system of a large proportion of assessed ASD patients (Singh et al., J. Biomed. 2002, Sci. 9: 359-364). In addition, significant increases in plasma levels of gamma interferon and IL-12 have been discovered in ASD patients when compared to non-ASD controls (Singh, 1996, J. Neuroimmunol. 66: 143-145). Further, a metabolite of nitrous oxide, nitrate, is appreciably higher in autistic children, and nitrate and gamma interferon levels are positively correlated with autism (Sweeten et al, 2004, Biol. Psychiatry, 55:434-437; US 2006/0182747 AI).

So, there is likelihood that the autism is partly an autoimmune disease or has autoimmune component. There is every possibility, more than the mercury in the vaccines; it is the antigenic component of the vaccine protein molecules that may trigger and induce autoimmune type of inflammatory reaction in the brain contributing to the ASD. Because of the autoantibodies component of the disease, treat the autism patients with intranasal administration of monoclonal antibodies (mAB) along with insulin instilled to ORE and external ear. We have used Etanercept as one of the Monoclonal antibody.
I. Place ASD patient as described above.
II. Administer insulin 4 IU each through the nostril to ORE and 5 mg (or higher doses) of Etanercept in each nose. It can be administered to the ear as described also in addition to the ORE.
III. The Etanercept is reconstituted with dilutant and use 5 mg in each ear. The dose can be increased as we see the positive results. Etanercept is marketed as a lyophylized powder in 25 mg and 50 mg vials which must be reconstituted with a diluents.
IV. Let the patient stay in the neck extended position for 15-20 minutes to allow the absorption of therapeutic agents without dripping back into the nasopharynx.
V. Thirty to sixty minutes later, subject the patients to various physical, speech and other therapies prescribed as part of the protocol.
VI. Because the mAB is used intranasally and delivered to ORE, not systemically, the dangers of developing tumors and aggravation of existing infections are minimal. If the child is suffering from viral or other infection, do not initiate the treatment till the infection is cleared. If the patient is suffering from any tumors, do not initiate this treatment using mAB.

### Example 6

Prophylaxis against ASD after vaccination: Up to now, there is no prophylaxis or method to abort autism development after vaccination when prodromal symptoms of autism develop or elaborate. If care givers or parents notice the development of ASD like prodromal symptoms after vaccination in the child which previously did not have any such symptoms, it could be conceived as due to production of cytokines in the CNS as reaction to protein components of the vaccination. In such cases, the following method of therapy can be used to curtail or cure the condition or stop from further advancement by elimination inflammatory cytokines which can contribute to the full blown disease.
I. Place ASD patient as described above.
II. Administer insulin 4 IU each through the nostril to ORE and 5 mg of Etanercept.
III. Administer the Etanercept on the external ear also as discussed in the above examples. Use 5 mg doses to each ear.
IV. The Etanercept is reconstituted with dilutant and use 25 microgram/kg in each ear. The dose can be increased as we see the positive results. Etanercept is marketed as a lyophilized powder in 25 mg and 50 mg vials which must be reconstituted with a diluent.
V. Make sure there are no signs or symptoms of hypoglycemia.
VI. Let the patient stay in the neck extended position for 15-20 minutes to allow the absorption of therapeutic agents without dripping back into the nasopharynx.
VII. Thirty to sixty minutes later, subject the patients to various physical, speech and other therapies prescribed as part of the protocol.
VIII. This procedure is carried out for about 2-3 weeks or until the symptoms clear.

This treatment can be carried out till the symptoms disappear.

### Example 7

Given the complex unknown pathophysiology of ASD, it may be naive to hope for a single “magic bullet” that will result in neuroprotection, rescue of damaged neurons and restore CNS function to normal levels. It is possible that the development of the ASD is due to trauma to the brain during intrauterine life, birth, and post vaccination. It can easily be labeled as a chronic Posttraumatic stress disorder (PTSD) of children. PTSD is a severe anxiety disorder that can develop after exposure to any event those results in psychological trauma. Can the vaccination be a trauma event triggering the disease? Based on numerous experimental studies (Syeed and Stein 2009), progesterone given to both males and females can have an impact in ASD similar to PTSD as describe below:
a. Progesterone cross the BBB and reduce brain edema for the treatment of stroke or traumatic brain injury (TBI).
b. It reduces lipid ROS mediated peroxidation damage which plays a part in post-injury ischemic conditions; generates metabolites which reduce pro-apoptotic and increase anti-apoptotic enzymes.
c. The expression of pro inflammatory genes and their protein products reduced with lower cytokines in the CNS after traumatic brain injury (TBI) thus protecting neuropil from further damage.
d. Progesterone is said to influence the expression of aquaporins (pores which drain edema fluids at the site of trauma such as concussion, stroke, may be even cytokine induced swelling) concerned in the resolution of the brain edema.
e. It safeguards neurons away from the site the injury which could be damage or die to the insult.
f. It has been shown to prevent prolonged vasoconstriction of the coronaries in premenopausal Rhesus monkeys.
g. It enhances oligodendroglia induced myelination in young and aged CNS of rats with myelinating disorders such as multiple sclerosis.
h. Progesterone protects and spares cognitive, sensory, and spatial learning performance in laboratory rats injury to the medial frontal cortex which is needed in ASD.
i. Its effects are replicated across species (mice, rats, cats, and humans) with equivalent effective doses. That means, it can be used to treat ASD in children.
j. Progesterone does block excitotoxicity by reducing GABA, but a compound with stronger NMDA and glutamate actions given in combination with progesterone may produce better synergistic effects. This can be easily adopted as describe in the examples.
   Hence, the progesterone leads to improvements via a variety of molecular mechanisms, making it likely that interacting pleiotropic actions are accountable for its observed benefits (Iqbal Sayeed and Donald G. Stein. Progesterone as a neuroprotective factor in traumatic and ischemic brain injury J. Verhaagell, el al. (Eds.) Progress in Brain Research. Vol. 175; 2009; Elsevier B. V. Pages 219-237). Hence, progesterone may have role in the treatment of ASD with insulin.
I. Place ASD patient as described above.
II. Administer insulin 4 IU each through the nostril to ORE and 2 to 5 mg of progesterone. If water soluble composition is formulated, it can be easily administered to ORE to be delivered to CNS. It can be used as a nasal spray combined with intranasal insulin delivered at ORE.
III. Nasal spray or water soluble preparations of progesterone are not available; use intravenous administration; then, 15 minutes later administer intranasal insulin at ORE.
IV. Administer the Etanercept on the external ear also as discussed in the above examples. Use 5 mg doses to each ear.
V. The Etanercept is reconstituted with dilutant and use 25 microgram/kg in each ear. The dose can be increased as we see the positive results. Etanercept is marketed as a lyophylized powder in 25 mg and 50 mg vials which must be reconstituted with a diluent.
VI. Make sure there are no signs or symptoms of hypoglycemia.
VII. Let the patient stay in the neck extended position for 15-20 minutes to allow the absorption of therapeutic agents without dripping back into the nasopharynx.
VIII. Thirty to sixty minutes later, subject the patients to various physical, speech and other therapies prescribed as part of the protocol.
IX. This procedure is carried out for about 2-3 weeks or till the symptoms clear.

The above described method can be used to treat all kinds of PTSD and stroke to save the brain from further trauma. We have used insulin, progesterone with hyperbaric therapy with dramatic relief of symptoms especially stroke patients. One of the patients started speaking after one week of treatment and gained more motor function.

### Example 8

US 2010/0130566 AI discloses the methods for treating ASD that comprises administering to a subject an agent that activates the Locus Coeruleus-Noradrenergic (LC-NA) system of the brain thereby treating autism spectrum disorders (ASD) in the subject. There is an endless list of LC-NA stimulating agent's which are incorporated herein. The systemic administration of these agents is fraught with secondary effects. These inventors do not discuss the use of insulin through ORE. The agents that activate the locus coeruleus-noradrenergic (LC-NA) system of the brain include, but are not limited to, adrenergic agonists, noradrenergic re-uptake inhibitors, agents that prevent or reduce degradation of noradrenalin, antagonists of pre-synaptic inhibition of noradrenergic nerve terminals, and epigenetic agents. The list of these therapeutic agents are as follows:
a) Examples of alpha agonists include, but are not limited to: phenylephrine, methoxamine, cirazoline and xylometazoline.
b) Examples of alpha2 agonists include, but are not limited to: dexmedetomidine, xylazine and tizanidine.
c) Examples of beta agonists include, but are not limited to: isoprenaline and dobutamine.
d) Examples of beta2 agonists include, but are not limited to: salbutamol (albuterol), bitolterol mesylate, formoterol, isoprenaline, levalbuterol, metaproterenol, salmeterol, terbutaline, and ritodrine.
e) Examples of beta3 agonists include, but are not limited to: L-796568 (Nisoli et al. 1996), amibegron and solabegron.
f) Examples of adrenergic reuptake inhibitors include, but are not limited to: 3,4-methylenedioxyamphetamine, amitriptyline, amoxapine, amphetamine, benzphetamine, dextroamphetamine, dothiepin, doxepin, duloxetine, imipramine, iprindole, maprotiline, mazindol, methamphetamine, milnacipran, n-methy 1-3,4-methy enedioxyamphetamine, nortriptyline, opipramol, protriptyline, reboxetine, reserpine, tetrabenazine, tomoxetine, trimipramine, tyramine and viloxazine.

With such a wide list, one can easily select the one most readily available in the market with least systemic effects which in liquid from to be administered to the ORE. Such a drug is Clonidine; used extensively for decades. It is a centrally acting α-adrenergic receptor agonist with more affinity for α₂ than α₁. Clonidine is used to treat anxiety, panic disorder, ADHD (FDA approved) insomnia; to ease withdrawal symptoms associated with the long-term use of narcotics, alcohol and nicotine (smoking) as well as for migraine headaches, hot flushes associated with menopause; psychiatric, stress, post-traumatic stress disorder, borderline personality disorder, Tourette's syndrome. Clonidine fools the brain into believing that catecholamine levels are higher than they really are, it causes the brain to reduce its signals to the adrenal medulla, which in turn lowers catecholamine production and blood levels. This is an ideal drug to be used for ASD who has many of the above described symptoms.
I. Place ASD patient as described above.
II. Administer insulin 4 IU each through the nostril to ORE with Clonidine 5 micrograms per kilogram of body weight per day. This can be combined with administration of Clonidine to the external ear.
III. Make sure there are no signs or symptoms of hypoglycemia.
IV. Let the patient stay in the neck extended position for 15-20 minutes to allow the absorption of therapeutic agents without dripping back into the nasopharynx.
V. Thirty to sixty minutes later, subject the patients to various physical, speech and other therapies prescribed as part of the protocol.

### Example 9

Researchers have investigated prenatal testosterone levels in mothers of children who develop autistic spectrum disorders. Manning et al. examined 72 children with autism, including 23 children with Asperger's Syndrome (i.e. these children have less serve autistic affects), 34 siblings, 88 fathers, 88 mothers, and sex and age-matched controls. These researchers demonstrated that the more severely affected the children were the higher the levels of prenatal testosterone (Manning. T, Baron-Cohen S, Wheelwright S, Sanders G. The 2nd to 4th digit ratio and autism. DevMed Child Neurol 2001; 43:160-4. Lutchmaya S, Baron-Cohen S, Raggatt P, Knickmeyer R, Manning. T. 2nd to 4th digit ratios, fetal testosterone and estradiol. Early Hum Dev 2004; 77:23-8). The breakdown products of testosterone are well known to play a role in male pattern baldness, in the development of the benign prostatic hypertrophy and prostate cancer. It is important to note that ratio of male to female autism is 4:1, indicating there is role of testosterone in production of autism in such high percentage of male children. Finasteride is an inhibitor of 5-alpha reductase by binding to 5-alpha reductase similar to testosterone, but with the effect of remaining bound to it rather than being converted, thereby blocking the space that testosterone would otherwise have taken. The FDA approved the drug Finasteride, which blocks the breakdown of testosterone into 5-alpha-di-hydro-testosterone, (DTH), has been shown to be highly effective in preventing and treating these conditions. That being the case, we want use Finasteride along with insulin administered intranasally to ORE.
I. Place ASD patient as described above.
II. Administer insulin 4 IU each through the nostril to ORE with 0.25-0.5 mg finasteride (Proscar and Propecia trade names). The liquid finasteride is prepared by dissolving 5 mg tablet in 5 ml of sterile saline. Filter it and each ml will contain one mg of the active ingredient. Use 0.25 to 0.5 ml of the preparation and mix with insulin preparation. Then, administer intranasally to ORE region as described above. If it cannot be prepared for intranasal administration, administer this therapeutic agent, orally as described above.
III. Make sure that there are no signs or symptoms of hypoglycemia.
IV. Let the patient stay in the neck extended position for 15-20 minutes to allow the absorption of therapeutic agents without dripping back into the nasopharynx.
V. Thirty to sixty minutes later, subject the patients to various physical, speech and other therapies prescribed as part of the protocol.

### Example 10

U.S. Pat. No. 5,225,407 discloses a method for the treatment of autism or other disorders originating in childhood in which there is mental retardation which comprises administering to a human subject an effective doses of a compound which acts as an antagonist of 5-hydroxytryptamine (5-HT′) at 5-HT3 receptors administered orally or parentarily. They do not describe intranasal ORE administration with insulin as described in this invention. Purdon, et al. showed anti serotonergic pharmacotherapy has been partially effective in treating a subgroup of children with autistic disorder. They describe where the two patients underwent psychiatric and neuropsychological examination before and after treatment with risperidone, a potent 5-HT2 antagonist with additional D2 antagonistic properties who showed improvements despite long histories of cognitive compromise and high likelihood of damage to the central nervous system (Purdon S E, Lit W, Labelle A, Jones B D. Risperidone in the treatment of pervasive developmental disorder. Can J Psychiatry. 1994 September; 39(7):400-5). Subsequent studies by Shea, et al. (Pediatr Neuro/1994, 11:89) showed risperidone-treated patients who were taking risperidone (mean dosage: 0.04 mg/kg/day; 1.17 mg/day) showed 87% global improvement in their condition compared with the placebo group (40%). They concluded that this drug is well tolerated and efficacious in treating behavioral symptoms associated with PDD in children.
I. Place ASD patient as described above.
II. Administer the oral risperidone (mean dosage: 0.04 mg/kg/day; 1.17 mg/day), which easily crosses the BBB, at least 60 minutes before the administration of insulin and IGF-1 so that the oral anti serotonergic medications have reached the CNS crossing the BBB to exert their desired pharmacological effect. Then, administer insulin and IGF-1 ORE and auditory routes. The insulin will augment and amplify the effects effect of therapeutic agents circulating in the blood of the CNS after absorption through the oral administration, hence the dose may be curtailed by 50% of the original prescribed dose.
III. Administer insulin 4-6 IU through each of the nostril to ORE (depending on the age and weight of the ASD) two times a day through the specially designed catheter or dropper, as described above.
IV. Make sure there are no signs or symptoms of hypoglycemia.
V. Let the patient stay in the neck extended position for 15-20 minutes to allow the absorption of therapeutic agents without dripping back into the nasopharynx.
VI. Thirty to sixty minutes later, subject the patients to various physical, speech and other therapies prescribed as part of the protocol.

### Example 11

The compound 2,3:4,5-bis-0-(1-methylethylidene)-B-D-fructopyranose sulfamate known as topiramate has been demonstrated to be effective as adjuvant therapy or as mono therapy in treating simple partial seizures and secondarily generalized seizures (E. Faught, B. I. Wilder, R. E. Ramsey, R. A. Rei Fe, L D. Kramer, G. W. Pledger, R. M. Karim Et. Ai., Epilepsia 36 (S4) 33, 1995; S. K. Sachdeo, R. C. Sachdeo, R. A. Reife, P. Lim And G. Pledger, Epilepsia 36 (S4) 33, 1995). It is currently marketed for the treatment of simple and complex partial seizure epilepsy with or without secondary generalized seizures. Clinical studies on topiramate have revealed previously unrecognized pharmacological properties which suggest that topiramate will be effective in treating autism with a statistically noteworthy reduction of seizure. There is also known enhancement of GABA activity in the brain along with reduced glutamate receptor activity that are useful in the treatment of autism. It is available in 25,100 or 200 mg tablet to be taken orally.
I. Place ASD patient as described above.
II. Administer the oral topiramate, which easily cross the BBB, at least 60 minutes before the administration of insulin and IGF-1 so that the oral medications have reached the CNS crossing the BBB to exert their desired pharmacological effect. Then, administer insulin and IGF-1 ORE and auditory routes. The insulin will augment and amplify the effects effect of therapeutic agents circulating in the blood after absorption through the oral administration; hence, the dose may be curtailed by 50-80% of the original prescribed dose.
III. Administer insulin 2-4-6 IU through each of the nostril to ORE (depending on the age and weight of the ASD) two times a day through the specially designed catheter or dropper as described above.
IV. Make sure there are no signs or symptoms of hypoglycemia.
V. Let the patient stay in the neck extended position for 15-20 minutes to allow the absorption of therapeutic agents without dripping back into the nasopharynx
VI. Thirty to sixty minutes later, subject the patients to various physical, speech and other therapies prescribed as part of the protocol.

### Example 12

Since the finding in 1961 of elevated serotonin (5-hydroxytryptamine) levels in the blood of patients with autism; collective studies from behavioral neuroscience, platelet serotonin levels, pharmacologic, and genetic studies point toward the involvement of serotonin abnormalities in autistic disorder (Cook E H. Leventhal B L. The serotonin system in autism. Current Opinion in Pediatrics. 8 (4):348-354, 1996). Further, the majority of individuals with autism, who are treated with serotonin transporter inhibitors, have a reduction in ritualistic behavior and aggression. Reduction of central nervous system serotonin, induced by acute tryptophan depletion, causes a worsening of stereotyped behavior. Although the function of serotonin (5-hydroxytryptamine [5-HT]) in the central nervous system is still being elucidate, a variety of studies have indicated an important role for serotonin in central nervous system development, social behavior, sleep, aggression, anxiety, and affective regulation as observed in ASD. Therefore, it is not surprising that serotonin has been the most exhaustively studied neurochemical in autism over the past three decades.

The most compelling evidence for the relationship between serotonin levels and autism is the efficacy of antidepressant medications that inhibit serotonin transport. Potent serotonin transporter inhibitors include the tricyclic antidepressant clomipramine and the selective serotonin reuptake inhibitors fluoxetine, sertraline, fluvoxamine, and paroxetine. Serotonin transporter inhibitors have been shown to reduce rituals associated with anxiety and to reduce aggression in more than 50% of children. These drugs have also been used successfully to treat self-injurious behavior and stereotypic movements in patients with mental retardation without autism. The acute effect of administration of these drugs in healthy adults is a reduction in basolateral limbic system (amygdala and hippocampus) metabolism. Serotonin may have a role in the developmental neuropathologic abnormalities found in the hippocampus, amygdala, and cerebellum in ASD disorder (Bauman M, Kemper T: Neuroanatomic observations of the brain in autism. In The Neurobiology of Autism. Edited by Bauman M, Kemper T. Baltimore: johns Hopkins University Press; 1994:119-145). The serotonin transporter is expressed at all presynaptic serotonin terminals.

Fenfluramine acts through a neurotransmitter in the brain also called 5-HT or 5-hydroxyltryptmine. Fenfluramine (trade names Pondimin, Ponderax and Adifax) causes the release of serotonin by disrupting vesicular storage of the neurotransmitter, and reversing serotonin transporter function. The end result is a feeling of fullness and loss of appetite. Serotonin has received much study over the years because it plays a role in such things as regulation of mood; control of eating, sleeping, and arousal; and in the regulation of pain. Serotonin levels are abnormally high in some autistic children. Researchers theorized that the high serotonin level in autistic children was what caused them to display abnormal signs of mood, eating, low tolerances to pain, etc. Therefore, by decreasing the serotonin levels, one could ameliorate the symptoms of autism. Fenfluramine decreases the serotonin concentrations in the brain, therefore decreasing the symptoms of autism caused by serotonin. Most of the patients of the studies showed little benefit from this drug.

Periactin is a serotonin inhibitor, which means it has same effects as fenfluramine. This drug is normally used as an antihistamine; but, because of its additional affect on serotonin, has been tried on autistics. Periactin will have the same effects of ameliorating the symptoms caused by excess of serotonin. It another drug that decreases serotonin concentrations thus reduces some of the symptoms of autism.
I. Place ASD patient as described above.
II. Administer insulin 4 IU each through the nostril to ORE with any potent serotonin transporter inhibitors include the tricyclic antidepressant clomipramine or the selective serotonin reuptake inhibitors fluoxetine, sertraline, fluvoxamine, and paroxetine. If used oral used, cut down the dose by at least 50%. Any systemic therapeutic agents administered to intranasal ORE region should be reduced to 10% but no more than 50% of the oral dose. That is the standard guiding principle.
III. If the nasal ORE preparation cannot be made, and only oral therapeutic agents are available, administer the oral topiramate, which easily cross the BBB, at least 60 minutes before the administration of Insulin and IGF-1 so that the oral medications have reached the CNS crossing the BBB to exert their desired pharmacological effect. Then, administer insulin and IGF-1 via the ORE and auditory routes. The insulin will augment and amplify the effects effect of therapeutic agents circulating in the blood of the CNS after absorption through the oral administration, hence the dose may be curtailed by 50-80% of the original oral prescribed dose.
IV. Administer insulin 2-4-6 IU through each of the nostril to ORE (depending on the age and weight of the ASD) one to two times a day through the specially designed catheter or dropper, as described above.
V. Make sure there are no signs or symptoms of hypoglycemia.
VI. Let the patient stay in the neck extended position for 15-20 minutes to allow the absorption of therapeutic agents without dripping back into the nasopharynx.
VII. Thirty to sixty minutes later, subject the patients to various physical, speech and other therapies prescribed as part of the protocol.

### Example 13

Proopiomelanocortin (POMC) is the precursor for Vendorphin, and is synthesized in discrete areas of the brain such as the basal ganglia, cortex, and amygdala, the hypothalamus and the pituitary. The basal ganglia are located deep within the brain and are very important in motor activities. The cortex is found on the outside of the brain and receives sensory inputs, sends out motor outputs, and also processes information. The amygdala is part of the limbic system and is therefore very important in memory, as well as emotions. The hypothalamus and pituitary are important for the overall regulation of the body through hormones and other molecules. Prodynorphin (PDYN) is the precursor for dynorphin, and is also found in many areas of the brain, as well as the spinal cord. In fact, drugs such as Naltrexone, which neutralize these receptors, thus preventing the binding of opioids or opiates, opiate like endogenous substance Prodynorphin have been shown to counteract the effects of morphine. Naltrexone has also been used in the treatment of autism with some success. Morphine is highly selective for the μ receptor. The enkephelins have been shown to act at the μ and δ receptors while dynorphin has been shown to act at the K receptor. These receptors are also located in areas that are important for the perception of pain (nociception).

Naltrexone is an opioid antagonist, which means that it inhibits or reduces the effects of opioids on the body. Children with autism who were studied were known to have similar symptoms to those who were addicted to opiates. Naltrexone reduces the effects of endogenous opioids. Effects of Naltrexone on hyperactivity, learning, and social behavior were reported for doses of 0.5 to 2.0 mg administered in single doses and at 24-, 48-, or 72-hour intervals. Naltrexone reduced restlessness and hyperactivity in autistic children with described improvement with Naltrexone on a well-established rating scale for autism without changes in eye contact or social proximity. Mild gastrointestinal symptoms, appetite decrease, and drowsiness may occur in these patients.
I. Place ASD patient as described above.
II. Administer insulin 4 IU each through the nostril to ORE with 0.025 mg of Naltrexone through the nostril to ORE.
III. Make sure there are no signs or symptoms of hypoglycemia.
IV. Let the patient stay in the neck extended position for 15-20 minutes to allow the absorption of therapeutic agents without dripping back into the nasopharynx.
V. Thirty to sixty minutes later, subject the patients to various physical, speech and other therapies prescribed as part of the protocol.

### Example 14

Studies show that the autistic adults given an intravenous doses of oxytocin had a statistically significant reduction in repetitive behaviors that are associated with autism (Hollander et al, American College of Neuropsychopharmacology Annual Meeting, December 2006; Neuropsychopharmacology (2006) 31, 1-11. doi:10.1038/sj.npp.1300880; published online 31 Aug. 2005). To give IV all the time is impractical. Hence, we want to our ORE instillation of this therapeutic agent. US 2010/0311655 AI discloses the use of Oxytocin to treat autism. They do not describe the use of these therapeutic agents with insulin to accelerate the relief of signs and symptoms in ASD through the nasal ORE route.

Oxytocin is a mammalian hormone secreted by the pituitary gland that acts as a neurotransmitter and it stimulates uterine contractions and milk let-down. A study on autistic children reported that such children had significantly lower levels of plasma oxytocin than normal children. Elevated oxytocin levels were associated with higher scores on social and developmental tests in non-autistic children. (Modahl, et al., Bioi. Psychiatric 43:270-277, 1998). A number of oxytocin analogs have been evaluated as possible substitute agents for inducing uterine contraction and milk let-down in mammalian patients with the goal of minimizing oxytocin's side effects. One such analog, carbetocin (I-butanoic acid-2-(O-methyl-L-tyrosine)-I-carbaoxytocin, or, alternatively, deamino-I monocarba-(2-O-methyltyrosine)-oxytocin [d(COMOT)]). The half-life of carbetocin is reportedly 4 to 10 times longer than that of oxytocin. An effective dose or multi-dose treatment regimen for the instant formulations will ordinarily be selected to approximate a minimal dosing regimen that is necessary and sufficient to substantially prevent or alleviate autism spectrum disorders, related disorders and/or symptoms of such disorders in the subject. An effective treatment regime may also involve prophylactic dosage administered on a day or multi-dose per day basis lasting over the course of days, weeks, months or even years. The effectiveness can be demonstrated according to a variety of methods described above.

Oxytocin injection (USP Pitocin) is a sterile, clear, colorless aqueous solution of synthetic oxytocin without impurities such as vasopressin (ADH) of natural extracted ones, for intravenous infusion or intramuscular injection. It is standardized to contain 10 units of oxytocic hormone/mL; has the empirical formula C₄₆H₆₆N₁₂O₁₂S₂, with molecular weight 1007.19. Because of its low molecular weight, it is easily absorbed by ORE and transported to the CNS in the treatment of autism. The dose used intranasally to ORE is so small, that serious side effects such as: allergic are minimal.
I. Place ASD patient as described above.
II. Administer insulin 4 IU each through the nostril to ORE with 1 or 2 units of the oxytocin hormone depending upon the age and weight. Do not use spray to prevent its effect, systemically.
III. Make sure there are no signs or symptoms of hypoglycemia.
IV. Let the patient stay in the neck extended position for 15-20 minutes to allow the absorption of therapeutic agents without dripping back into the nasopharynx.
V. Thirty to sixty minutes later, subject the patients to various physical, speech and other therapies prescribed as part of the protocol.

### Example 15

Melatonin is a natural hormone, synthesized and released by the pineal gland (FIG. 15) at the base of the brain, above the superior colliculus. In a cyclical manner, it regulates our built in biological clock. The Pineal gland is outside of the blood-brain barrier and is part of the CVO. Melatonin production is inhibited by light and allowed by darkness which induces sleep, hunger, etc. at set times, and regulate our body functions. Now, there is some anecdotal evidence which shows that melatonin may have some behavior modification role in autism. The resultant higher levels of the hormone in blood inhibit the centers in the brain stem responsible for keeping us awake; thus, inducing sleeps.

Melatonin increases proliferation of cultured neural stem cells obtained from mice nervous tissue. Melatonin is involved in energy metabolism and body weight control in small animals. Cancer patients using melatonin found a reduced incidence of death. In animal models, melatonin has been shown to ameliorate glutamate-induced neuronal death; presumed due to its antioxidant effects. In a clinical safety study involving 31 ALS patients, high-dose rectal melatonin (300 mg/day for 2 years) was shown to be tolerated well with beneficial effects.

Individuals with ASD may have lower than normal levels of melatonin. A 2008 study found that unaffected parents of individuals with ASD also had lower melatonin levels, and that the deficits were associated with low activity of the ASMT gene, which encodes the last enzyme of melatonin synthesis (Melke, j; Goubran Botros, H; Chaste, P; Betancur, C; Nygren, G; Anckarsäter, H; Rastam, M; Ståhlberg, 0, et al. (2007). “Abnormal melatonin synthesis in autism spectrum disorders”. Molecular Psychiatry 13 (1): 90-8).

Multiple studies have demonstrated that 2 to 10 mg of melatonin may benefit children with ASD who have trouble falling asleep and/or maintaining sleep. A small 2011 randomized crossover trial found that the administration of melatonin, when compared to placebo, decreased sleep latency and increased total sleep time, but had no effect on the number of night time awakenings (Wright, Barry; Sims, David; Smart, Siobhan; Alwazeer, Ahmed; Alderson-Day, Ben; Allgar, Victoria; Whitton, Clare; Tomlinson, Heather, et al. (2010). “Melatonin Versus Placebo in Children with Autism Spectrum Conditions and Severe Sleep Problems Not Amenable to Behaviour Management Strategies: A Randomized Controlled Crossover Trial”. journal of Autism and Developmental Disorders 41 (2): 175-84). At this time, though there is no guidelines exist for the use of melatonin in children with ASD, but it can be added as part of the treatment protocol.
I. Place ASD patient as described above.
II. Administer the oral Melatonin, which easily cross the BBB, at least 60 minutes before the administration of Insulin and IGF-1 so that the oral medications have reached the CNS crossing the BBB to exert their desired pharmacological effect.
III. Then, administer insulin and IGF-1 ORE and auditory routes. The insulin will augment and amplify the effects effect of therapeutic agents circulating in the blood of the CNS after absorption through the oral administration; hence, the dose may be curtailed by 50-80% of the original prescribed dose.
IV. Administer insulin 4-6 IU through each of the nostril to ORE (depending on the age and weight of the ASD) two times a day through the specially designed catheter or dropper as described above.
V. Make sure there are no signs or symptoms of hypoglycemia.
VI. Let the patient stay in the neck extended position for 15-20 minutes to allow the absorption of therapeutic agents without dripping back into the nasopharynx.
VII. Thirty to sixty minutes later, subject the patients to various physical, speech and other therapies prescribed as part of the protocol.

### Example 16

It has been discovered that administering the proper dose of an of a NMDA-receptor antagonist or a pharmaceutically acceptable salt, thereof appears to significantly improve frontal executive functions associated with autistic symptoms, including, but not limited to, speech expression and decreased perseveration. It is said to act by reducing the neuronal signal-to-noise ratio as one of the mechanism of action in ASD. Furthermore, administering such a NMDA-receptor antagonist or a pharmaceutically acceptable salt thereof has not been shown to cause side-effects associated with medications previously used to treat the symptoms of autism.

The drug, Memantine belongs to a class of drugs called NMDA receptor antagonists, which help reduce abnormal activity in the brain by binding to NMDA receptors on brain cells and blocking the activity of the neurotransmitter glutamate. At normal levels, glutamate aids in memory and learning, but if levels are too high, glutamate over stimulate nerve cells, killing off key brain cells.

Memantine is the first in a novel class of Alzheimer's disease medications acting on the glutamatergic system by blocking NMDA glutamate receptors which help reduce abnormal activity in the brain by binding to NMDA receptors on brain cells and blocking the activity of the neurotransmitter glutamate. At normal levels, glutamate aids in memory and learning, but if levels are too high, glutamate over stimulate nerve cells, killing the key brain cells. In addition, memantine acts as an agonist at the dopamine D2 receptor.

Memantine has recently been approved by FDA for the treatment of memory loss in Alzheimer's disease, a neurodegenerative disorder of the nervous system. This approval was based on three randomized placebo-controlled trials that showed significant improvements in cognitive, functional and global endpoints in this population (Tariot et al., JAMA. 2004; 291:317-24, Reisberg et al, N Engl J Med., April 3; 348(14): 1333-41 (2003), Winblad et al., Int J Geriatr Psychiatry, 14(2): 135-46 (1999)). Similar results were seen in two trials in vascular dementia (Wilcock et al., Int Clin Psychopharmacol., 17(6): 297-305 (2002), Orgogozo et al., Stroke, 33: 1834-9 (2002)).

Autism is nothing like Alzheimer's disease, and it is a neuro-developmental disorder rather than neuro-degenerative Alzheimer's-type disease. There are currently no drugs approved for the treatment of autism and other PDDs. Serotonin reuptake inhibitors have been shown to have some effect on repetitive behaviors. Atypical antipsychotics seem to be effective in the treatment of aggression. Antiepileptic medications may be useful for aggression, especially in children with epileptiform abnormalities. Amantadine, a weak inhibitor of the NMDA glutamate receptor, has been tested in autism. The study showed some improvement in irritability and hyperactivity; however, amantadine has a very weak affinity for this receptor and therefore very high doses would be required to get an adequate effect. Memantine has moderate affinity for the NMDA receptor and has properties such as rapid blocking/unblocking abilities that render it very well tolerated.

U.S. Pat. No. 7,456,224 B2 discloses the use of memantine for the treatment of autism although they do not disclose its use with intranasally ORE route administered with insulin. It is therefore an object of the present invention, to provide a method for treating autism via administering an effective dose of a NMDA-receptor antagonist or a pharmaceutically acceptable salt thereof in combination of insulin.
I. Place ASD patient as described above.
II. Administer the oral memantine (in the dose of 5 mg, or 10 mg which easily cross the BBB, at least 60 minutes before the administration of Insulin and IGF-1 to the ORE and ear, so that the oral medications have reached the CNS crossing the BBB to exert their desired pharmacological effect.
III. Then, administer insulin and IGF-1 ORE and auditory routes. The insulin will augment and amplify the effects effect of therapeutic agents circulating in the blood of the CNS after absorption through the oral administration, hence the dose may be curtailed by 40-80% of the original prescribed dose.
IV. Administer insulin 4-6 IU through each of the nostril to ORE (depending on the age and weight of the ASD) two times a day through the specially designed catheter or dropper as described above.
V. Make sure there are no signs or symptoms of hypoglycemia.
VI. Let the patient stay in the neck extended position for 15-20 minutes to allow the absorption of therapeutic agents without dripping back into the nasopharynx.
VII. Thirty to sixty minutes later, subject the patients to various physical, speech and other therapies prescribed as part of the protocol.
   According to studies, this therapy ranged from between 8 to 40 weeks, with an average duration of therapy of 18 weeks and an average daily dosage of 8.1 mg.

### Example 17

US 201110130390 AI describe USE OF COX-2 inhibitors for the treatment of schizophrenia, delusional disorders, affective disorders, tic disorders and autism. The preferred COX-2 inhibitors for the use according to the present invention include celecoxib (Celebrex®), rofecoxib (Vioxx®), meloxicam, valdecoxib, etoricoxib. Celecoxib can be administered at a dose of 50-1600 mg per day. Celecoxib is used in the form of tablets (Celebrex®) for oral administration. But, they do not describe the use of ORE insulin to augment and amplify the effects circulating cox-2 inhibitors in the brain which our invention describes. It is known that the activation of COX-2 mediates inflammatory processes. COX-2 is expressed in brain tissue and activated by cytokines like IL-2, IL-6 and IL-10, and cytokine-activated COX-2 expression mediates further inflammatory processes. The COX-2 inhibitors belong to the class of non-steroidal anti-inflammatory drugs (NSAIDs). It has been known for some time that many of the common NSAIDs modulate prostaglandin synthesis by inhibition of Cyclooxygenase that catalyze the transformation of arachidonic acid—the first step in the prostaglandin synthesis. The term COX-2 inhibitor include compounds which selectively inhibit cyclooxygenase-2 over Cyclooxygenase-I, and also includes pharmaceutically acceptable salts, thereof. In the cerebrospinal fluid of schizophrenic patients, maybe even autism patient, there is the increase of the cytokines in the CNS may be accompanied by increased COX-2. The effectiveness of COX-2 inhibitors is based on the finding that celecoxib down-regulates the cytokine induced CNS COX-2 activation.
I. Administer maximum dose of selected COX-2 inhibitor, orally with plenty of water. Wait for at least 60 minutes for these therapeutic agents to be absorbed and circulated in the CNS. Celecoxib is used in the form of tablets (Celebrex®) for oral administration is our choice.
II. Place ASD patient as described above.
III. Administer insulin 4 IU each through the nostril to ORE IV. Let the patient stay in the neck extended position for 15-20 minutes to allow the absorption of therapeutic agents without dripping back into the nasopharynx.
V. Thirty to sixty minutes later, subject the patients to various physical, speech and other therapies prescribed as part of the protocol.

### Example 18

U.S. Pat. No. 7,276,492 B2 discloses the use of angiotensin converting enzyme inhibitors (ACE inhibitors) in patients suffering from endogenous behavioral disorders, manifested as behavioral improvements in people, both children and adults, who suffer from endogenous disorders including attention deficit disorder (ADD), obsessive compulsive disorder (OCD), oppositional explosive defiant disorder (OED), anxiety and panic disorders (APD), and impulsive temper, rage and outburst behavior disorder (TROBD). These endogenous behavioral disorder may be one which is associated with the elevation of blood pressure. They do not describe the use of these therapeutic agents for the treatment of autism and the above described conditions in combination with insulin as described in the present invention.

Some of the above signs and symptoms are akin to ASD, and overlap. The action of an ACE inhibitor or ACE receptor blockers, by reducing conversion of angiotensin I, relieves vasoconstriction and reduces aldosterone secretion. It also provides negative feedback on renin release, which is also believed to decrease aldosterone secretion. Another possible basis for the effectiveness for ACE inhibitors in the treatment of ASD, is that ACE is identical to bradykininase (kininase II), which acts on bradykinin. Bradykinin stimulates prostaglandin biosynthesis and it is believed that ACE inhibitors also inhibit bradykininase and thereby increase bradykinin levels. ACE inhibitors thus stimulate the biosynthesis of prostaglandin, which is a vasodilator and may contribute to the pharmaceutical effects of ACE inhibitor. The present invention provides a method for the treatment of patients suffering from an endogenous behavioral disorder by administering to the patient a therapeutic quantity of at least one ACE inhibitor, such as a dicarbocyl containing ACE inhibitor, e.g., lisinopril. As used herein, and in the claims, “endogenous” behavioral disorders are disorders not associated with or resulting from traumatic head injury or other trauma, but rather those which find their etiology solely in non-trauma induced conditions such as psychological conditions or illnesses, hormonal imbalances, or the like.
I. Administer maximum dose of selected dicarbocyl-containing ACE inhibitor (lisinopril), 10 mg per day, oral administration with plenty of water. Wait for at least 60 minutes for these therapeutic agents to be absorbed and circulated in the CNS.
II. Place ASD patient as described above.
III. Administer insulin 4 IU each through the nostril to ORE
IV. Let the patient stay in the neck extended position for 15-20 minutes to allow the absorption of therapeutic agents without dripping back into the nasopharynx.
V. Thirty to sixty minutes later, subject the patients to various physical, speech and other therapies prescribed as part of the protocol.

### Example 19

Sibutramine hydrochloride monohydrate (N,N-Dimethyl-1-[1-4-chlorophenyl cyclobutyl]-3-methylbutylamine hydrochloride monohydrate) is available as MERIDIA®. It has been prescribed for the treatment of obesity, depression, diabetic hyperglycemia, hyper lipidemia, senile dementia and related conditions, and Parkinson's disease. It has been discovered that sibutramine can be effectively used in treating other problems unrelated to these conditions. Sibutramine acts by inhibiting the reuptake of norepinephrine, serotonin and dopamine thereby intensifying their effects in the brain. Capsules are presently available with discrete dosages of 5, 10 and 15 mg for oral administration. Interestingly, the lowest effective dose has been shown to be 0.25 mg daily and the highest dose has been shown to be 45 mg (15 mg 3 times daily) indicating its broad therapeutic index and allows more flexibility in dosing. It is important to note that the signs and symptoms for which it was given were alleviated within minutes or hours of treatment, and the improvements continued long term.

Further, the known effects of sibutramine on serotonin, dopamine and norepinephrine activation, is believed to be an effect of this medication on the endorphic opiate neurotransmitter system which is evident in the patients who have been relieved of pain, rage, anger, self-mutilation and particularly narcotic addicts. It is thought that the self-injury appears to activate the opioid system and the relief through sibutramine strongly suggests endorphic involvement. Hence, the sibutramine activates endorphins and/or, modifies the endorphinergic opioid systems to promote serenity and lack of pain and stress, and to reduce craving in the addicts. Finally, a differentiation between the serotonin, dopamine and norepinephrine potentiation versus the opiate like therapeutic effects of sibutramine may be made in terms of duration of action, whereas the serotonin, dopamine, norepinephrine agonist effects lasted in most patients for only 4-6 hours, the opiate effects appear to last all day and require only a single morning dosage. This will be great benefit in treatment of ASD. We have used this medication in many pain patients who are drug dependent and have psychological problems. Besides prescribing to control the weight, we have used this therapeutic agent for treating severe atypical psychosis in cancer and Lyme disease patients, drug addict, for rage, violence, self-abuse, post-traumatic stress disorder (PTSD, GWS), cognitive and sexual dysfunction, attention deficit disorder, psychoses with delusions, hallucinations, fatigue, sleep disorders, fibromyalgia, chronic fatigue syndrome, Reflex Sympathetic Dystrophy Syndrome (RSDS), also known as Complex Regional Pain Syndrome (CRPS). Lyme disease and cancer patients responded well with intranasal insulin with sibutramine. This drug may be well suited to be used in ASD children with our invention to treat neurological, behavioral, and cognitive symptoms or disorders emanating from primary organic impairments.
I. Administer the oral sibutramine in doses of 2.5-5 mg oral which easily cross the BBB in the morning.
II. Wait at least 60 minutes before the administration of Insulin and IGF-1 so that the oral medications have reached the CNS crossing the BBB to exert their desired pharmacological effect.
III. Place ASD patient as described above.
IV. Then, administer insulin and IGF-1 ORE and auditory routes. The insulin will augment and amplify the effects effect of therapeutic agents circulating in the blood of the CNS after absorption through the oral administration; hence, the dose may be curtailed by 50-80% of the original prescribed dose.
V. Administer insulin 4-6 IU through each of the nostril to ORE (depending on the age and weight of the ASD) two times a day through the specially designed catheter or dropper as described above.
VI. Make sure there are no signs or symptoms of hypoglycemia.
VII. Let the patient stay in the neck extended position for 15-20 minutes to allow the absorption of therapeutic agents without dripping back into the nasopharynx.

As the effect of sibutramine lasts for a day, the dose need not be given till the next day, although one may administer insulin through ORE twice a day. The insulin will augment and amplify the effects of sibutramine in the CNS, hence the dose of sibutramine can be lower.

Numerous modifications; adjuvants, alternative arrangements of steps explained and examples given, herein may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form function and manner of procedure, assembly and use may be made. While the preferred embodiment of the present invention has been described, it should be understood that various changes, adaptations and modifications may be made, thereto. It should be understood, therefore, that the invention is not limited to details of the illustrated invention.

What is claimed is:

1. A method of treating autism spectrum disorders (autism-ASD) consists of administering to a patient an effective dose of insulin and their pharmaceutically acceptable salt thereof, with adjuvant therapeutic agents, wherein the insulin administered in a dosage:
a. ranging from approximately 2 to 4 or 6 units depending upon the age and weight of the patients,
b. is delivered directly to the olfactory region of both nostrils,

2. The method of treating autism spectrum disorders (autism-ASD) according to claim 1 wherein said dose is delivered by using specially designed catheter, dropper, and catheter with balloon system.

3. The method of treating autism spectrum disorders (autism-ASD) according to claim 1 wherein said dose is delivered to the external auditory meatus to the subject.

4. The method of treating autism spectrum disorders (autism-ASD) according to claim 1 further comprising placing an ASD patient in the hyperbaric chamber.

5. The method of treating autism spectrum disorders (autism-ASD) according to claim 1 wherein is delivered to the external auditory meatus to the subject; the formulation further comprising an IGF-1.

6. The method of treating autism spectrum disorders (autism-ASD) according to claim 1 further comprising an adjuvant therapeutic agent IGF-1.

7. The method of treating autism spectrum disorders (autism-ASD) according to claim 1, wherein the treatment ameliorates one or more of ASD signs and symptoms such as irritability, hyperactivity, inattention, stereotypy, inappropriate speech, impaired social interaction, impaired communication, restricted interests and repetitive behavior.

8. A method for transporting insulin to the brain of a mammal comprising: applying a pharmaceutical composition comprising the insulin to an upper third of a nasal cavity deposited on the olfactory region (ORE) of the mammal roof of the nose (cribriform plate of the ethmoid bone region), wherein the insulin and other adjuvant therapeutic agents are absorbed through a nasal ORE transported to the brain of the mammal in the treatments of autism.

9. The method of claim 1, wherein the various known adjuvant therapeutic agents, as well as other pharmaceutical, biochemical, nurticeuticals, and biological agents or compounds composition used to treat ASD, comprises a liquid, a powder, a spray, a nose drop, a gel, an infusion, or a combination, thereof.

10. The method of treating autism spectrum disorders (autism-ASD) according to claim 1 further comprising an adjuvant therapeutic agent monoclonal antibodies (mAB) administered to ORE and external ear.

11. The method of treating autism spectrum disorders (autism-ASD) according to claim 1 further comprising an adjuvant therapeutic agent which is an anti-convulsant, topiramate.

12. The method of treating autism spectrum disorders (autism-ASD) according to claim 1 further comprising an adjuvant therapeutic agent which is a stimulant medication, methylphenidate, atomoxetine, Dexamphetamine, and amphetamine.

13. The method of treating autism spectrum disorders (autism-ASD) according to claim 1 further comprising an adjuvant therapeutic agent which is a immunotherapeutic agent such as Monoclonal antibodies (mAB).

14. The method of treating autism spectrum disorders (autism-ASD) according to claim 1 further comprising an adjuvant therapeutic agent which is a sibutramine.

15. The method of treating autism spectrum disorders (autism-ASD) according to claim 1 further comprising an adjuvant therapeutic agent which is a Clonidine.

16. The method of treating autism spectrum disorders (autism-ASD) according to claim 1 further comprising an adjuvant therapeutic agent which is a risperidone and finasteride.

17. The method of treating autism spectrum disorders (autism-ASD) according to claim 1 further comprising an adjuvant therapeutic agents which is 5HT-3 receptor blockers.

18. The method of treating autism spectrum disorders (autism-ASD) according to claim 1 further comprising an adjuvant therapeutic agent which is a progesterone.

19. The method of treating autism spectrum disorders (autism-ASD) according to claim 1 further comprising an adjuvant therapeutic agent which is a Naltrexone.

20. The method of treating autism spectrum disorders (autism-ASD) according to claim 1 further comprising an adjuvant therapeutic agent which is a Oxytocin.
